(19) United States
(12) Patent Application Publication
(10) Pub. No.: US 2017/0067909 A1
(43) Pub. Date: Mar. 9, 2017

(54) ASSAY-READY RECOMBINANT CELLS TRANSIENTLY OVEREXPRESSING GENES ENCODING DRUG TRANSPORTER PROTEINS AND/OR DRUG METABOLIZING ENZYMES

(71) Applicant: Corning Incorporated, Corning, NY (US)

(72) Inventors: Na Li, Winchester, MA (US); Jie Wang, Winchester, MA (US); Christopher J. Patten, Scituate, MA (US)

(21) Appl. No.: 15/269,045

(22) Filed: Sep. 19, 2016

Related U.S. Application Data
(63) Continuation-in-part of application No. 15/163,218, filed on May 24, 2016.
Application No. 15/163,218 is a continuation of application No. 14/972,012, filed on Dec. 16, 2015.
Application No. 14/972,012 is a division of application No. 14/644,000, filed on Mar. 10, 2015, now abandoned.
Application No. 14/644,000 is a continuation of application No. PCT/US2013/059152, filed on Sep. 11, 2013.
(60) Provisional application No. 61/699,466, filed on Sep. 11, 2012.

(51) Int. Cl. G01N 33/68; C07K 14/705; G01N 33/50; A01N 1/02

(52) U.S. Cl. CPC G01N 33/6872; A01N 1/0284; C07K 14/705; G01N 33/5044; G01N 33/5067

(57) ABSTRACT

Recombinant cells including one or more transiently overexpressed genes encoding a drug transporter protein, wherein the recombinant cell is cryopreserved and activity of the drug transporter protein is detectable in a population of the recombinant cells prior to cryopreservation at an uptake ratio of at least 5. Processes of preparing cryopreserved transiently transfected recombinant cells, including transiently transfecting cells with one or more genes encoding a drug transporter protein and cryopreserving the transiently transfected recombinant cells within 48 hours of transfection. A population of the transiently transfected recombinant cells transiently overexpress the one or more genes encoding the drug transporter protein at a detectable level prior to cryopreserving and the detectable level is an uptake ratio of at least 5.

### CROSS-REFERENCE TO RELATED APPLICATIONS

This application is filed under 35 U.S.C. §111 as a continuation-in-part application of U.S. application Ser. No. 15/163,218, filed on May 24, 2016, which is a continuation application of U.S. application Ser. No. 14/972,012, filed on Dec. 16, 2015, which is a division of U.S. application Ser. No. 14/644,000, filed on Mar. 10, 2015, which is a continuation application of International Application No. PCT/US2013/059152, filed on Sep. 11, 2013, which designates the United States and claims priority to U.S. Provisional Patent Application No. 61/699,466, filed on Sep. 11, 2012, the contents of which are hereby incorporated by reference in their entirety.

### BACKGROUND

Field

The present disclosure relates to assay-ready preparations of recombinant cells including one or more transiently overexpressed genes encoding a drug transporter protein and/or a drug metabolizing enzyme, to processes of preparing cryopreserved, transiently transfected recombinant cells, and to suspension assays for assessing activity of drug transporter proteins and/or a drug metabolizing enzymes of recombinant cells.

Technical Background

Drug development is a costly and time consuming process of identifying, characterizing, and proving the safety and efficacy of drug candidates. One reason is that drug candidates must satisfy certain safety and efficacy criteria established by government agencies, such as, e.g., the U.S. Food and Drug Administration and European Medicines Agency, to market and sell new drugs. To study the safety of drugs, assays are conducted to screen drug candidates to determine whether they have an effect on drug transporter proteins and/or drug metabolizing enzymes (such as, e.g., whether the drug candidates are substrates or inhibitors thereof). This is because drug transporter proteins and/or drug metabolizing enzymes have an established role in the absorption, distribution, metabolism, and/or elimination of drugs. Specifically, drug candidates (or metabolites of drug candidates) that significantly affect drug transporter proteins and/or drug metabolizing enzymes may also produce undesirable toxicity and/or drug-drug interactions, reducing the safety profile thereof.

Another reason that drug development is costly and time consuming is that drug transporters are genetically polymorphic, which is one of the major causes of differences in drug efficacy, safety, and pharmacokinetic variation in different individuals and populations. Therefore, the importance of genetic variations in drug transporters for drug disposition and response has been increasingly recognized in the past decade. The drug transporter organic anion transporting polypeptide 1B1 (OATP1B1) is genetically polymorphic and plays a major role in hepatic uptake of a variety of clinically important drugs. Two common single nucleotide polymorphisms (c.388A>G and c.521T>C) have been reported in OATP1B1 wth altered functionality. Compared to the wild-type allele OATP1B1*1 (c.388A and c.521T), the two haplotypes OATP1B1*5 (c.388A and c.521C) an OATP1B1*15 (c.388G ad c.521C) are consistently associated with reduced transporting activity. For example, and with respect to the *15 haplotype, the effect on drug disposition was evidenced by increased statin AUC (“area under the curve) in individuals carrying the 521CC genotype (Niemi M, Pharmacol Rev. (63):157 (2011).

Additionally, the frequencies of OATP1B1 genetic variants show marked ethnic differences. Predicting the pharmacokinetic effect of these genetic variants on drug disposition is critical for understanding the inter-individual variations in drug efficiary and safety.

Although cryopreserved cell lines transiently expressing a gene encoding a drug transporter protein and/or drug metabolizing enzyme are available for drug screening assays, such as, e.g., Corning®TransportoCells™ available from Corning Life Sciences (Bedford, Mass.), such cryopreserved recombinant cells are not assay-ready. Rather, such cryopreserved recombinant cells require users to thaw, plate, and culture the recombinant cells prior to performing a drug screening assay. Thawing, plating, and culturing the recombinant cells may take a user at least 24 hours to complete, increasing both the cost and time required to perform critical drug screening assays.

Accordingly, ongoing needs exist for assay-ready recombinant cells transiently expressing genes encoding a drug transporter protein and/or a drug metabolizing enzyme.

### SUMMARY

In embodiments, a recombinant cell including one or more transiently transfected overexpressed genes encoding a drug transporter protein is disclosed. The recombinant cell is cryopreserved and activity of the drug transporter protein is detectable in a population of the recombinant cells prior to cryopreservation at an uptake ratio of at least 5.

In other embodiments, a process of preparing cryopreserved transiently transfected recombinant cells is disclosed. The process includes transiently transfecting cells with one or more genes encoding a drug transporter protein to provide the transiently transfected recombinant cells, and cryopreserving the transiently transfected recombinant cells within 48 hours of transfection. A population of the transiently transfected recombinant cells transiently overexpress the one or more genes encoding the drug transporter protein at a detectable level prior to cryopreserving the transiently transfected recombinant cells. The detectable level prior to cryopreserving is an uptake ratio of at least 5.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a graph of the percentage of viable cells from cell stock, cells after electroporation (hereinafter, “EP”) and cells after thaw from cryopreservation for FreeStyle™ 293-F (hereinafter, “FS293”) cells and 293-F cells grown in suspension.

FIG. 2 are images of transfected cells 4 hrs (A), 24 hrs (B) and 48 hrs (C) after plating following thaw from cryopreservation.

FIG. 3 are images of 293-F cells transfected with pOATP1B1 expression plasmid plated at (A) 0.4×106 viable cells per well and (B) 0.2×106 viable cells per well in 24-well poly-D-lysine coated plates and cultured in plating media at 24 hrs post-plating (following thaw from cryopreservation).

FIG. 4 are images of 293-F cells transfected with MATE1, MATE2K OATP1B3, long isoform OAT1 (full length cDNA with 563 amino acids; hereinafter, “OAT1 long”), short isoform OAT1 (missing 13 amino acid at C-terminus 522-534, with 550 amino acids; hereinafter, “OAT1 short”), OAT3, and pCMV vector plated at 0.4×106 cells per well in 24-well poly-D-lysine coated plates at 24 hrs post-plating (following thaw from cryopreservation).

FIG. 5 are fluorescence images of adhered HEK293 cells transfected with 50 μg/ml, 100 μg/ml or 200 μg/ml green fluorescent protein (GFP) 24 hrs (A) and 48 hrs (B) following EP.

FIG. 6 is a graph of the percentage of viable cells following EP of adhered HEK293 cells using varying amounts of DNA.

FIG. 7A is a graph of estradiol-17β-glucuronide (i.e., E17βG) uptake activity following various incubation times in adhered HEK293 cells transfected with varying amounts of DNA (i.e., 0, 50 μg/ml, 100 μg/ml, 200 μg/ml or 400 μg/ml OATP2/OATP1B1) at 48 hrs post EP.

FIG. 7B is a graph of estradiol-17β-glucuronide (i.e., E17βG) uptake activity following various incubation times in adhered HEK293 cells transfected with varying amounts of DNA (i.e., 0, 50 μg/ml, 100 μg/ml, 200 μg/ml or 400 μg/ml OATP2/OATP1B1) at 96 hrs post EP.

FIG. 8 is a graph of signal to noise ratio of estradiol-17β-glucuronide (i.e., E17βG) uptake following various incubation times in adhered HEK293 cells transfected with varying amounts of DNA (i.e., 0, 50 μg/ml, 100 μg/ml, 200 μg/ml or 400 μg/ml OATP2/OATP1B1) at 48 hrs post EP.

FIG. 9 is a graph of estradiol-17β-glucuronide (i.e., E17βG) uptake activity in adhered HEK293 cells transfected with either OATP2/OATP1B1 using a small scale EP device (OC400), OATP2/OATP1B1 using a large scale EP device (CL2), or an empty vector control.

FIG. 10 is a graph of signal to noise ratio of estradiol-17β-glucuronide (i.e., E17βG) uptake following various incubation times in adhered HEK293 cells transfected with OATP1B1 gene using either “Control” (i.e., traditional lipid transfection reagent (lipofectamine 2000, available from Invitrogen)) or STX, MaxCyte scalable EP device.

FIG. 11 is a graph of signal to noise ratio of estradiol-17β-glucuronide (i.e., E17βG) uptake following various incubation times in adhered HEK293 cells transfected with OATP1B1 that are freshly plated or plated following thaw from cryopreservation.

FIG. 12 are images of HEK293 cells transfected with OATP1B1*1a (Gene Accession No. NM_006446.4), OATP1B1*1b (Gene Accession No. NM_006446.3), OATP1B3, pCMV vector, long isoform OAT1 (full length cDNA with 563 amino acids), OAT3, OCT1 or OCT2 using MaxCyte scalable EP device and scale-up process followed by cryopreservation, thawing, plating on Poly-D-Lysine plates and incubation for 24 hrs post-plating.

FIG. 13A is a graph depicting results of a time-dependent assay of p-Aminohippuric acid (i.e., PAH) (prototypical substrate for OAT1) uptake in HEK293 cells overexpressing OAT1 or pCMV vector following various incubation times (i.e., 1, 2, 5, 10 and 15 min.) with PAH at a concentration of 3 μM.

FIG. 13B is a graph depicting results of a kinetic assay whereby uptake of PAH at a concentration in the range of 3 to 200 μM was measured in HEK293 cells overexpressing OAT1 following incubation for 5 min. Km and Vmax, calculated using Sigma-plot, are shown as insert in the graph.

FIG. 13C is a graph depicting results of an inhibition assay whereby HEK293 cells overexpressing OAT1 were incubated with PAH at a concentration of 15 μM and probenecid (i.e., an OAT1 inhibitor) at a concentration in the range of 0-300 μM for 5 min. 1050, calculated using Sigma-plot, is shown as insert in the graph.

FIG. 14A is a graph depicting results of a time-dependent assay of Estrone-3-sulfate (i.e., E3S) (prototypical substrate for OAT3) uptake in HEK293 cells overexpressing OAT3 or pCMV vector following various incubation times (i.e., 1, 2, 5, 10 and 15 min.) with E3S at a concentration of 1 μM.

FIG. 14B is a graph depicting results of a kinetic assay whereby uptake of E3S at a concentration in the range of 0.5 to 32 μM was measured in HEK293 cells overexpressing OAT3 following incubation for 1 min. Km and Vmax, calculated using Sigma-plot, are shown as insert in the graph.

FIG. 14C is a graph depicting results of an inhibition assay whereby HEK293 cells overexpressing OAT3 were incubated with E3S at a concentration of 4 μM and probenecid (i.e., an OAT3 inhibitor) at a concentration in the range of 0-300 μM for 5 min. 1050, calculated using Sigma-plot, is shown as insert in the graph.

FIG. 15A is a graph depicting results of a time-dependent assay of TEA (i.e., a prototypical substrate for OCT1) uptake in HEK293 cells overexpressing OCT1 or pCMV vector following various incubation times (i.e., 1, 2, 5, 10 and 15 min.) with TEA at a concentration of 31 μM.

FIG. 15B is a graph depicting results of a time-dependent assay of metformin (i.e., a prototypical substrate for OCT1) uptake in HEK293 cells overexpressing OCT1 or pCMV vector following various incubation times (i.e., 1, 2, 5, 10 and 15 min.) with metformin at a concentration of 3.8 μM.

FIG. 15C is a graph depicting results of a concentration-dependent assay whereby uptake of TEA at a concentration of 1, 10 and 100 μM was measured in HEK293 cells overexpressing OCT1 or pCMV vector following incubation for 10 min.

FIG. 15D is a graph depicting results of a concentration-dependent assay whereby uptake of metformin at a concentration of 0.1, 1 and 10 μM was measured in HEK293 cells overexpressing OCT1 or pCMV vector following incubation for 10 min.

FIG. 15E is a graph depicting results of an inhibition assay whereby HEK293 cells overexpressing OCT1 were incubated with TEA and OCT1 inhibitor (i.e., quinidine, verapamil or decynium-22) at various concentrations in the range of 0.1-500 μM for 10 min.

FIG. 15F is a graph depicting results of an inhibition assay whereby HEK293 cells overexpressing OCT1 were incubated with metformin at a concentration of 3.8 μM and OCT1 inhibitor cimetidine at various concentrations in the range of 4 μM to 3 mM for 10 min.

FIG. 16A is a graph depicting results of a time-dependent assay of TEA (i.e., a prototypical substrate for OCT2) uptake in HEK293 cells overexpressing OCT2 or pCMV vector following various incubation times (i.e., 1, 2, 5, 10 and 15 min.) with TEA at a concentration of 31 μM.

FIG. 16B is a graph depicting results of a time-dependent assay of metformin (prototypical substrate for OCT2) uptake in HEK293 cells overexpressing OCT2 or pCMV vector following various incubation times (i.e., 1, 2, 5, 10 and 15 min.) with metformin at a concentration of 3.8 μM.

FIG. 16C is a graph depicting results of a concentration-dependent assay whereby uptake of TEA at a concentration of 1, 10 and 100 μM was measured in HEK293 cells overexpressing OCT2 or pCMV vector following incubation for 10 min.

FIG. 16D is a graph depicting results of a concentration-dependent assay whereby uptake of metformin at a concentration of 0.1, 1 and 10 μM was measured in HEK293 cells overexpressing OCT2 or pCMV vector following incubation for 10 min.

FIG. 16E is a graph depicting results of an inhibition assay whereby HEK293 cells overexpressing OCT2 were incubated with metformin at a concentration of 3.8 μM and OCT2 inhibitor cimetidine at a concentration in the range of 4 μM to 3 mM for 10 min. 1050, calculated using Sigma-plot, is shown as insert in the graph.

FIG. 17A is a graph depicting results of a time-dependent assay of estradiol-17β-glucuronide (i.e., E17βG) uptake in HEK293 cells overexpressing OATP1B1*1a or pCMV vector following various incubation times (i.e., 1, 2, 5, 10 and 15 min.) with E17βG at a concentration of 1 μM.

FIG. 17B is a graph depicting results of a time-dependent assay of estrone-3-sulfate (i.e., E3S) uptake in HEK293 cells overexpressing OATP1B1*1a or pCMV vector following various incubation times (i.e., 1, 2, 5, 10 and 15 min.) with E3S at a concentration of 1 μM.

FIG. 17C is a graph depicting results of a time-dependent assay of rosuvastatin uptake in HEK293 cells overexpressing OATP1B1*1a or pCMV vector following various incubation times (i.e., 1, 2, 5, 10 and 15 min.) with rosuvastatin at a concentration of 1 μM.

FIG. 17D is a graph depicting results of a concentration-dependent assay whereby uptake of E17βG at a concentration in the range of 0.25 to 40 μM was measured in HEK293 cells overexpressing OATP1B1*1a following incubation for 1 min. Km and Vmax, calculated using Sigma-plot, are shown as insert in the graph.

FIG. 17E is a graph depicting results of a concentration-dependent assay whereby uptake of rosuvastatin at a concentration in the range of 0.78 to 50 μM was measured in HEK293 cells overexpressing OATP1B1*1a following incubation for 5 min. Km and Vmax, calculated using Sigma-plot, are shown as insert in the graph.

FIG. 17F is a graph depicting results of a concentration-dependent assay whereby uptake of E17βG at a concentration of 1 μM was measured in HEK293 cells overexpressing OATP1B1*1a following incubation with inhibitor cyclosporin A at a concentration in the range of 0.04 to 30 μM for 5 min. 1050, calculated using Sigma-plot, is shown as insert in the graph.

FIG. 18A is a graph depicting results of a time-dependent assay of E17βG uptake in HEK293 cells overexpressing OATP1B1*1b or pCMV vector following various incubation times (i.e., 1, 2, 5, 10 and 15 min.) with E17βG at a concentration of 1 μM.

FIG. 18B is a graph depicting results of a time-dependent assay of E3S uptake in HEK293 cells overexpressing OATP1B1*1b or pCMV vector following various incubation times (i.e., 1, 2, 5, 10 and 15 min.) with E3S at a concentration of 1 μM.

FIG. 18C is a graph depicting results of a time-dependent assay of rosuvastatin uptake in HEK293 cells overexpressing OATP1B1*1b or pCMV vector following various incubation times (i.e., 1, 2, 5, 10 and 15 min.) with rosuvastatin at a concentration of 1 μM.

FIG. 18D is a graph depicting results of a concentration-dependent assay whereby uptake of E17βG at a concentration in the range of 0.25 to 40 μM was measured in HEK293 cells overexpressing OATP1B1*1b following incubation for 1 min. Km and Vmax, calculated using Sigma-plot, are shown as insert in the graph.

FIG. 18E is a graph depicting results of an inhibition assay whereby uptake of E17βG at a concentration of 1 μM was measured in HEK293 cells overexpressing OATP1B1*1b following incubation with inhibitor cyclosporin A at a concentration in the range of 0.04 to 30 μM for 5 min. 1050, calculated using Sigma-plot, is shown as insert in the graph.

FIG. 19A is a graph depicting results of a time-dependent assay of cholecystokinin (i.e., CCK-8) uptake in HEK293 cells overexpressing OATP1B3 or pCMV vector following various incubation times (i.e., 1, 2, 5, 10 and 15 min.) with CCK-8 at a concentration of 1 μM.

FIG. 19B is a graph depicting results of a time-dependent assay of E17βG uptake in HEK293 cells overexpressing OATP1B3 or pCMV vector following various incubation times (i.e., 1, 2, 5, 10 and 15 min.) with E17βG at a concentration of 1 μM.

FIG. 19C is a graph depicting results of a concentration-dependent assay whereby uptake of CCK-8 at a concentration in the range of 0.5 to 20 μM was measured in HEK293 cells overexpressing OATP1B3 following incubation for 1 min. Km and Vmax, calculated using Sigma-plot, are shown as insert in the graph.

FIG. 19D is a graph depicting results of a concentration-dependent assay whereby uptake of rosuvastatin at a concentration in the range of 0.78 to 50 μM was measured in HEK293 cells overexpressing OATP1B3 following incubation for 5 min. Km and Vmax, calculated using Sigma-plot, are shown as insert in the graph.

FIG. 19E is a graph depicting results of an inhibition assay whereby uptake of CCK-8 at a concentration of 1 μM was measured in HEK293 cells overexpressing OATP1B3 following incubation with inhibitor cyclosporin A at a concentration in the range of 0.04 to 30 μM for 2 min. 1050, calculated using Sigma-plot, is shown as insert in the graph.

FIG. 20 is a flow chart of a suspension assay employing a centrifugation method or a vacuum manifold for characterizing activity of a drug transporter protein according to embodiments of this disclosure.

FIG. 21 is a flow chart of a suitable suspension assay employing a centrifugation method for characterizing activity of a drug transporter protein according to embodiments of this disclosure.

FIG. 22A is a bar graph of Corning® TransportoCells™ cells cultured in Erlenmeyer shaker flasks (i.e., Shaker Flask), Corning® BioCoat™ PDL 175 cm² Rectangular Straight Neck Cell Culture Flasks with Vented Cap (i.e., PDL-T175) or Falcon® 175 cm² Rectangular Straight Neck Cell Culture Flasks with Vented Cap (i.e., TC-T175) with respect to Viability at Harvest (%).

FIG. 22B is a bar graph of Corning® TransportoCells™ cells cultured in Erlenmeyer shaker flasks (i.e., Shaker Flask), Corning® BioCoat™ PDL 175 cm² Rectangular Straight Neck Cell Culture Flasks with Vented Cap (i.e., PDL-T175) or Falcon® 175 cm² Rectangular Straight Neck Cell Culture Flasks with Vented Cap (i.e., TC-T175) with respect to Average Fold of Cell Doubling (X).

FIG. 23A is a graph of Cell Density Per Well (K/well) of Corning® TransportoCells™ cells cultured in Erlenmeyer shaker flasks (i.e., SF), Corning® BioCoat™ PDL 175 cm² Rectangular Straight Neck Cell Culture Flasks with Vented Cap (i.e., PDL), or Falcon® 175 cm² Rectangular Straight Neck Cell Culture Flasks with Vented Cap (i.e., TC) with respect to Uptake Activity (pmol/mg/min).

FIG. 23B is a graph of Cell Density Per Well (K/well) of Corning® TransportoCells™ cells cultured in Erlenmeyer shaker flasks (i.e., SF), Corning® BioCoat™ PDL 175 cm² Rectangular Straight Neck Cell Culture Flasks with Vented Cap (i.e., PDL), or Falcon® 175 cm² Rectangular Straight Neck Cell Culture Flasks with Vented Cap (i.e., TC) with respect to Uptake Ratio. The positive control (cells cultured in Erlenmeyer shaker flask, 300K/well) in this experiment exhibited an uptake ratio (i.e., S/N) of 24 with substrate.

FIG. 24 is a bar graph of positive control cells (i.e., Control: OATP1B1 Cells), negative control cells (i.e., Neg Control), or HEK293 cells cultured in Erlenmeyer shaker flasks (i.e., Susp: CD_shaker flask), Corning® BioCoat™ PDL 175 cm² Rectangular Straight Neck Cell Culture Flasks with Vented Cap (i.e., Attached: Plating_PDL), Falcon® 175 cm² Rectangular Straight Neck Cell Culture Flasks with Vented Cap treated with plating media (i.e., Attached: Plating_TC), or Falcon® 175 cm² Rectangular Straight Neck Cell Culture Flasks with Vented Cap treated with CD_10% FBS (i.e., Attached: CD/FBS_TC) for 48 hours and supplemented with sodium butyrate with respect to Uptake Activity (pmol/mg/min).

FIG. 25A is a graph of Culture Time (hours) of HEK293 cells cultured in Erlenmeyer shaker flasks (i.e., Susp: CD_Shaker Flask) or in Corning® BioCoat™ PDL 175 cm² Rectangular Straight Neck Cell Culture Flasks with Vented Cap (i.e., Attached: PM_PDL) supplemented with sodium butyrate with respect to Cell Doubling.

FIG. 25B is a graph of Culture Time (hours) of HEK293 cells cultured in Erlenmeyer shaker flasks (i.e., Susp: CD_Shaker Flask) or in Corning® BioCoat™ PDL 175 cm² Rectangular Straight Neck Cell Culture Flasks with Vented Cap (i.e., Attached: PM_PDL) supplemented with sodium butyrate with respect to Uptake Activity (pmol/mg/min).

FIG. 26 is a bar graph of HEK293 cells cultured in Erlenmeyer shaker flasks (i.e., Susp: CD_Shaker Flask) and of HEK293 cells cultured in Corning® BioCoat™ PDL 175 cm² Rectangular Straight Neck Cell Culture Flasks with Vented Cap (i.e., Attached: PM_PDL) with and without the addition of sodium butyrate 24 hours prior to harvest with respect to Uptake Activity Immediately Post-Thawing (pmol/mg/min).

FIG. 27A is a bar graph of positive control cells (i.e., Control: OATP1B1 Cells), negative control cells (i.e., Neg Control), HEK293 cells cultured in Erlenmeyer Shaker Flasks (i.e., Susp: CD_shaker flask), in Corning® BioCoat™ PDL 175 cm² Rectangular Straight Neck Cell Culture Flasks with Vented Cap (i.e., Attached: Plating_PDL), in Falcon® 175 cm² Rectangular Straight Neck Cell Culture Flasks with Vented Cap treated with plating media (i.e., Attached: Plating_TC), or in Falcon® 175 cm² Rectangular Straight Neck Cell Culture Flasks with Vented Cap treated with CD_10% FBS (i.e., Attached: CD/FBS_TC) with respect to Uptake Activity (pmol/mg/min), wherein activity was assessed via Suspension Assay at 0 hours post-thaw, Suspension Assay at 1 hour post-thaw, or Plate Assay at 4 hours post-thaw.

FIG. 27B shows an image of confluency of HEK293 cells at the 4-hour plate assay, wherein the cells were cultured in Erlenmeyer Shaker Flasks.

FIG. 27C shows an image of concluency of HEK 293 cells at the 4-hour plate assay, wherein the cells were cultured in Corning® BioCoat™ PDL 175 cm² Rectangular Straight Neck Cell Culture Flasks with Vented Cap.

FIG. 28 is a bar graph of positive control cells (i.e., Control: OATP1B1 Cells), HEK293 cells cultured in Erlenmeyer Shaker Flasks (i.e., Susp: CD_shaker flask), in Corning® BioCoat™ PDL 175 cm² Rectangular Straight Neck Cell Culture Flasks with Vented Cap (i.e., Attached: Plating_PDL), in Falcon® 175 cm² Rectangular Straight Neck Cell Culture Flasks with Vented Cap treated with plating media (i.e., Attached: Plating_TC), or in Falcon® 175 cm² Rectangular Straight Neck Cell Culture Flasks with Vented Cap treated with CD_10% FBS (i.e., Attached: CD/FBS_TC) with respect to Uptake Ratio, wherein activity was assessed via Suspension Assay at 0 hours post-thaw, Suspension Assay at 1 hour post-thaw, or Plate Assay at 4 hours post-thaw.

FIG. 29A is a bar graph of positive control cells (i.e., Control: OATP1B1 Cells), negative control cells (i.e., Neg Control), HEK293 cells cultured in Erlenmeyer Shaker Flasks (i.e., Susp: CD_SF), in Corning® BioCoat™ PDL 175 cm² Rectangular Straight Neck Cell Culture Flasks with Vented Cap (i.e., Atta: Plating_PDL), in Falcon® 175 cm² Rectangular Straight Neck Cell Culture Flasks with Vented Cap treated with plating media (i.e., Atta: Plating_TC), or in Falcon® 175 cm² Rectangular Straight Neck Cell Culture Flasks with Vented Cap treated with CD_10% FBS (i.e., Atta: CD/FBS_TC) with respect to Viability (%), wherein HEK293 cells were thawed in Plating Media or in HBSS Buffer.

FIG. 29B is a graph of Viability (%) with respect to Thawing Media (i.e., HBSS Buffer or Plating Media) as described in FIG. 29A.

FIG. 30 is a flow chart of suitable culturing conditions according to embodiments of this disclosure.

FIG. 31A is a bar graph showing the uptake of E17βG in the presence and absence of sodium butyrate (“SB”) in HEK-293 cells that overexpressed monkey Oatp1b1, dog Oatp1b4, and rat Oatp1b2, as compared to human OATP1B1*1a (i.e., wild-type).

FIG. 31B is a bar graph showing the uptake of rosuvastatin in the presence and absence of sodium butyrate (“SB”) in HEK-293 cells that overexpressed monkety Oatp1b1, dog Oatp1b4, and rat Oatp1b2, as compared to human OATP1B1*1a (i.e., wild-type).

FIG. 32 is a graph of the time-dependent uptake of the probe substrate via OATP/Oatps. Uptake of 2.0 μM estradiol-17β-glucuronide in human OATP1B1*1a, monkey Oatp1b1, dog Oatp1b4, and rat Oatp1b2 cells were determined at 1, 2, 5, 10, and 15 minutes, respectively at 37° C.

FIG. 33 is a graph of Km values of E17βG in HEK-293 cells overexpressing monkey Oatp1b1, dog Oatp1b4, and rat Oatp1b2 (following incubation of 5 minutes). Km values were calculated according to Michaelis-Menten kinetics.

FIG. 34A is a graph of the species differences of human OATP1B1*1a and OATP1B3, monkey Oatp1b1, dog Oatp1b4, and rat Oatp1b2 substrate specificity for prototypical estradiol-17β-glucuronide.

FIG. 34B is a graph of the species differences of human OATP1B1*1a and OATP1B3, monkey Oatp1b1, dog Oatp1b4, and rat Oatp1b2 substrate specificity for prototypical estrone-3-sulfate.

FIG. 34C is a graph of the species differences of human OATP1B1*1a and OATP1B3, monkey Oatp1b1, dog Oatp1b4, and rat Oatp1b2 substrate specificity for prototypical CCK-8.

FIG. 34D is a species differences of human OATP1B1*1a and OATP1B3, monkey Oatp1b1, dog Oatp1b4, and rat Oatp1b2 substrate specificity for pitavastatin.

FIG. 34E is a species differences of human OATP1B1*1a and OATP1B3, monkey Oatp1b1, dog Oatp1b4, and rat Oatp1b2 substrate specificity for atorvastatin.

FIG. 34F is a species differences of human OATP1B1*1a and OATP1B3, monkey Oatp1b1, dog Oatp1b4, and rat Oatp1b2 substrate specificity for pravastatin.

FIG. 34G is a species differences of human OATP1B1*1a and OATP1B3, monkey Oatp1b1, dog Oatp1b4, and rat Oatp1b2 substrate specificity for simvastatin.

FIG. 35A is a graph depicting the results of a kinetic assay whereby uptake of estradiol-17β-glucuronide in human OATP1B1*1a, monkey Oatp1b1, dog Oatp1b4, and rat Oatp1b2 was measured after a 2-minute incubation at 37° C.

FIG. 35B is a table of the calculated results of the kinetic assays depicted in FIG. 35A.

FIG. 35C s a graph depicting the results of a kinetic assay whereby uptake of rosuvastatin in human OATP1B1*1a, monkey Oatp1b1, dog Oatp1b4, and rat Oatp1b2 was measured after a 2-minute incubation at 37° C.

FIG. 35D is a table of the calculated results of the kinetic assays depicted in FIG. 35C.

FIG. 35E is a graph depicting the results of a kinetic assay whereby uptake of atorvastatin in human OATP1B1*1a, monkey Oatp1b1, dog Oatp1b4, and rat Oatp1b2 was measured after a 2-minute incubation at 37° C.

FIG. 35F is a table of the calculated results of the kinetic assays depicted in FIG. 35E.

FIG. 36A is a graph depicting the IC₅₀ values of human OATP1B1*1a, monkey Oatp1b1, dog Oatp1b4, and rat Oatp1b2 determined by co-incubating the cells with 1 μM E17βG with cyclosporin A at a range of concentrations.

FIG. 36B is a graph depicting the IC₅₀ values of human OATP1B1*1a, monkey Oatp1b1, dog Oatp1b4, and rat Oatp1b2 determined by co-incubating the cells with 1 μM rosuvastatin with gemfibrozil at a range of concentrations.

FIG. 37A is a table depicting results of thawing and recovery of OATP1B1*5 and OATP1B1*15 HEK-293 cells.

FIG. 37B is a table depicting results of thawing and recovery, as well as Uptake Ratio of OAT2, OAT4, OCTN2 HEK-293 cells using the probe substrate lised in the table.

FIG. 37C is a tabe depicting results of thawing and recovery, as well as Uptake Ratio, of monkey Oatp1b1, dog Oatp1b4, and rat Oatp1b2 HEK-293 cells using E17βG.

FIG. 37D are images of the FIG. 20A HEK-293 cells transfected with OATP1B1*5 and OATP1B1*15, plated at 0.4×10⁶ cells per well in 24-well poly-D-lysine coated plates at 24 hrs post-plating (following thaw from cryopreservation).

FIG. 38A is a graph of the results of uptake assays conducted on OATP1B1*1a, OATP1B1*5, OATP1B1*15 and control cells for fluorescein methotrexate (F-MTX).

FIG. 38B is a graph of the results of uptake assays conducted on OATP1B1*1a, OATP1B1*5, OATP1B1*15 and control cells for E17βG.

FIG. 38C is a graph of the results of uptake assays conducted on OATP1B1*1a, OATP1B1*5, OATP1B1*15 and control cells for atorvastatin.

FIG. 38D is a graph of the results of uptake assays conducted on OATP1B1*1a, OATP1B1*5, OATP1B1*15 and control cells for simvastatin.

FIG. 38E is a graph of the results of uptake assays conducted on OATP1B1*1a, OATP1B1*5, OATP1B1*15 and control cells for pitavastatin.

FIG. 38F is a graph of the results of uptake assays conducted on OATP1B1*1a, OATP1B1*5, OATP1B1*15 and control cells for and fluvastatin.

FIG. 39A is a graph of the results of kinetic assays conducted on OATP1B1*1a, OATP1B1*5, OATP1B1*15 and control cells for E17βG.

FIG. 39B is a graph of the results of kinetic assays conducted on OATP1B1*1a, OATP1B1*5, OATP1B1*15 and control cells for pitavastatin.

FIG. 39C is a graph of the results of kinetic assays conducted on OATP1B1*1a, OATP1B1*5, OATP1B1*15 and control cells for rosuvastatin.

FIG. 39D is a table of the calculated results of the kinetic assays depicted in FIGS. 39A-39F.

FIG. 40 is a graphic illustration of the LC-MS/MS mediated targeted protein quantification process used.

FIGS. 41A-41D are graphs of the results of extract ion chromatograms of selected reaction monitoring for AQUA® peptide (Sigma-Aldrich) and samples of CORNING® TRANSPORTOCELLS™ OATP1B1*1a, control cells, OATP1B1*5 and OATP1B1*15 prepared according to the graphic illustration of FIG. 40. The lined arrow represents the peak for the signature peptide for OATP1B1 and the solid arrow represents the peak for the internal standard.

FIG. 42A is a graph of the DNA concentration necessary to achieve consistent expression across OATP1B1 wild-type and single nucleotide polymorphisms.

FIG. 42B is a graph showing the consistency of uptake activity and ratios across four different wild-type OATP1B1 lots.

FIG. 42C is a graph showing the consistency of protein expression across a control, three different wild-type OATP1B1 lots, a OATP1B1*5 lot and a OATP1B1*15 lot.

### DETAILED DESCRIPTION

As used herein the following terms shall have the definitions set forth below.

As used herein, the term “cell” includes both primary cells as well as established cell lines (e.g., human embryonic kidney HEK293 cells, Chinese hamster ovary CHO, Madin-Darby Canine Kidney Cells MDCK, Pig Kidney Epithelial Cells LLC-PK1, human epithelial colorectal adenocarcinoma cells Caco-2 and Chinese hamster lung fibroblast V79 cells).

As used herein, the term “drug transporter protein” refers to a membrane bound transport protein that includes, but is not limited to, ATP binding cassette (hereinafter, “ABC”) transporters and solute carrier (hereinafter, “SLC”) transporters.

As used herein, the term “drug metabolizing enzyme” includes, but is not limited to, cytochromes such as cytochromes (i.e., CYPs) P450; UDP-glucouronyl transferases (i.e., Uridine 5′-diphospho-glucuronosyltransferase) and other non-CYP drug metabolizing enzymes such as alcohol dehydrogenases, monoamine oxidases and aldehyde oxidases.

As used herein, the term “detectable” means that the activity of a selected probe substrate in cells transfected with a drug transporter protein and/or drug metabolizing enzyme shall be higher than the activity of the same probe substrate in cells transfected with empty vector; desirably, the difference in activity will be at least 5-fold.

As used herein, the use of upper case letters in transporter nomenclature identifies the human protein/gene, i.e., MRP2/ABCC2, etc.; smaller case letters indicate the transporter derives from a preclinical (i.e., nonhuman mammalian) species, e.g., Mrp2/Abcc2, etc. Unless otherwise specified, a gene is derived from any species (e.g., human or other mammal).

As used herein, the terms “OATP1B1”, “OATP2”, and “SLCO 1B1” are interchangeable and refer to a human protein/gene that corresponds to the nonhuman protein/gene Oatp2. Unless noted otherwise, reference to OATP1B1 is to OATP1B1*1a.

As used herein, the terms “OAT1” and “SLC22A6” are interchangeable and refer to an organic anion transporter 1. Unless noted otherwise, reference to OAT1 is to the full length cDNA encoding with 563 amino acids (also referred to herein as “OAT1 long”).

As used herein, the term “SNP” means single nucleotide polymorphism(s).

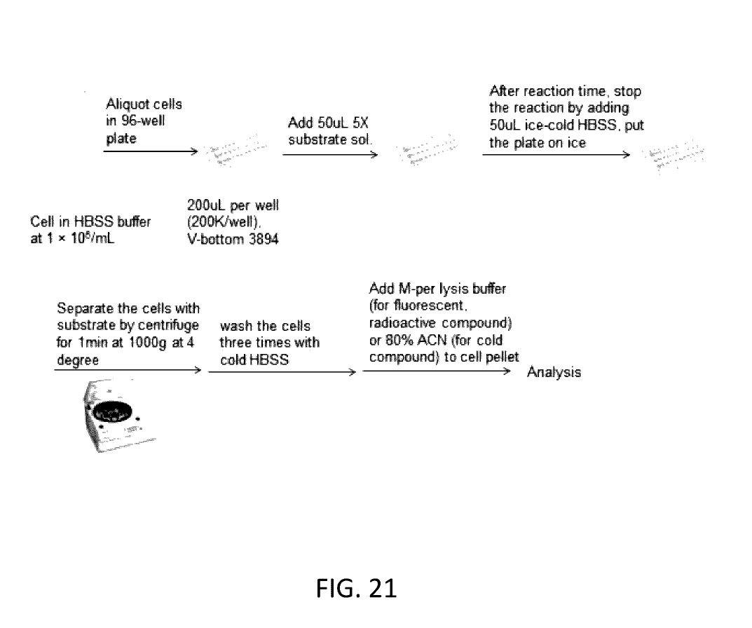

Reference will now be made in detail to embodiments of recombinant cells including one or more transiently overexpressed genes encoding a drug transporter protein, a drug metabolizing enzyme, or combination thereof. Thereafter, embodiments of processes of preparing cryopreserved, transiently transfected recombinant cells and suspension assays will be described in detail with specific reference to FIG. 21.

### I. Recombinant Cells

In embodiments, recombinant cells including one or more transiently overexpressed genes encoding a drug transporter protein, a drug metabolizing enzyme, or combination thereof, are disclosed. In embodiments, the recombinant cells are cryopreserved and activity of the drug transporter protein, drug metabolizing enzyme, or combination thereof, is detectable in a population of the recombinant cells prior to cryopreservation and/or following thaw from cryopreservation. In some embodiments, the recombinant cells are cryopreserved and activity of the drug transporter protein, drug metabolizing enzyme, or combination thereof, is detectable in a population of the recombinant cells prior to cryopreservation and/or following thaw from cryopreservation at an uptake ratio of at least 5 (i.e., 5:1). In some particular embodiments, the recombinant cells are cryopreserved and activity of the drug transporter protein, drug metabolizing enzyme, or combination thereof, is detectable in a population of the recombinant cells prior to cryopreservation at an uptake ratio of at least 5.

In embodiments, the recombinant cells are mammalian cells derived from a human or a non-human (e.g., mouse, rat, dog, monkey, hamster, and pig, etc.). In some embodiments, the recombinant cells are hepatocytes or endothelial cells. In some particular embodiments, the recombinant cells are hepatocytes. In other particular embodiments, the recombinant cells are established cells lines, such as, e.g., human embryonic kidney HEK293 cells.

In embodiments, the recombinant cells transiently overexpress one or more genes encoding a drug transporter protein, a drug metabolizing enzyme, or combination thereof. In some embodiments, the recombinant cells are transiently transfected with one or more genes encoding a drug transporter protein, a drug metabolizing enzyme, or combination thereof. In particular embodiments, the recombinant cells are transiently transfected as described subsequently with regard to processes of preparing cryopreserved, transiently transfected recombinant cells. In embodiments, the one or more transiently overexpressed genes is derived individually from a human or non-human (i.e., an animal) species. In some embodiments, the non-human species from which the one or more transiently overexpressed genes is derived are selected from the group consisting of a mouse, a rat, a dog, a monkey, a pig, and a guinea pig.

Human OATP1B1 and OATP1B3 are the two major OATP family members involved in hepatic uptake of numerous xenobiotics and drugs. Thus, there is much clinical evidence that both OATP1B1 and OATP1B3 are involved in DDI. Monkeys, dogs and rats are frequently used in preclinical studies to provide preclinical pharmacokinetics (i.e., ADME) as well as toxicity data for potential new drugs. Specifically, a recombinant model with overexpressed animal species Oatp proteins allows for the in vitro evaluation of substrate specificity and affinity, thereby facilitating the interpretation of potential interspecies differences in drug pharmacokinetic and toxicological responses.

A benefit of using monkey Oatp1b1 and 1b3 is the high degree of homology with the human counterparts; specifically, there is homology of approximately 91.9% between OATP1B1 and monkey Oatp1b1, and there is homology of approximately 93.5% between OATP1B3 and monkey Oatp1b3. Dog Oatp1b4 was cloned in 2010; since then, it has been determined that its expression level is the highest as compared to other Oatp family members. Rat Oatp1b2 is considered to be the rodent counterpart of the human OATP1B family.

In some embodiments, the recombinant cells include one or more transiently overexpressed genes encoding a drug transporter protein selected from the group consisting of ABC transporters, SLC transporters, and a combination thereof. In some particular embodiments, the recombinant cells include one or more transiently overexpressed genes encoding an ABC transporter. In embodiments, the human ABC transporter includes at least one of the proteins set forth in Table 1. Similarly, in embodiments, the one or more genes encoding the human ABC transporter include at least one of the genes set forth in Table 1.

TABLE 1

| | | ACCESSION |
|---|---|---|
| GENE NAME | PROTEIN NAME | NUMBER |
| MDR1/P-gp/ | Multidrug Resistance Protein 1 | NM_000927 |
| ABCB1 | | |
| MDR3/ABCB3 | Multidrug Resistance Protein 3 | NM_000443 |
| MRP1/ABCC1 | Multidrug Resistance Protein 1 | NM_004996 |
| MRP2/ABCC2 | Multidrug Resistance-Associated | NM_000392 |
| | Protein 2 | |
| MRP3/ABCC3 | Multidrug Resistance Protein 3 | NM_003786 |
| MRP4/ABCC4 | Multidrug Resistance Protein 4 | NM_005845 |
| MRP5/ABCC5 | Multidrug Resistance Protein 5 | NM_005688 |
| MRP6/ABCC6 | Multidrug Resistance Protein 6 | NM_001171 |
| MRP7/ABCC7 | Multidrug Resistance Protein 7 | NM_000492 |
| MRP8/ABCC8 | Multidrug Resistance Protein 8 | NM_000352 |
| BCRP/ABCG2 | Breast Cancer Resistance Protein | NM_004827 |
| BSEP/ABCB11 | Bile Salt Export Pump | NM_003742 |

In some particular embodiments, the recombinant cells include one or more transiently overexpressed genes encoding a human SLC transporter. In embodiments, the SLC transporter includes at least one of the proteins set forth in Table 2. Similarly, in embodiments, the one or more genes encoding the human SLC transporter include at least one of the genes set forth in Table 2.

TABLE 2

| | | ACCESSION |
|---|---|---|
| GENE NAME | PROTEIN NAME | NUMBER |
| OSTα | Organic Solute Transporter α | NM_152672 |
| OSTβ | Organic Solute Transporter β | NM_178859 |
| OATP1B1*/SLCO | Organic Anion-Transporting | NM_006446 |
| 1B1/OATP2 | Polypeptide 1B1 | |
| OATP1B3/ | Organic Anion-Transporting | NM_019844 |
| SLCO 1B3 | Polypeptide 1B3 | |
| OAT1/SLC22A6 | Organic Anion Transporter 1 | NM_004790 |
| OAT2/SLC22A7 | Organic Anion Transporter 2 | NM_006672 |
| OAT3/SLC22A8 | Organic Anion Transporter 3 | NM_004254 |
| OAT4/SLC22A11 | Organic Anion Transporter 4 | NM_018484 |
| OCT1/SLC22A1 | Organic Cation Transporter 1 | NM_003057 |
| OCT2/SLC22A2 | Organic Cation Transporter 2 | NM_003058 |
| OCT3/SLC22A3 | Organic Cation Transporter 3 | NM_021977 |
| OATP1A2/ | Organic Anion-Transporting | NM_134431 |
| SLCO1A2 | Polypeptide 1A2 | |
| OATP2B1/ | Organic Anion-Transporting | NM_007256 |
| SLCO2B1 | Polypeptide 2B1 | |
| PEPT1/SLC15A1 | Peptide Transporter 1 | NM_005073 |
| PEPT2/SLC15A2 | Peptide Transporter 2 | NM_021082 |
| OCTN1/SLC22A4 | Organic Cation/Ergothioneine | NM_003059 |
| | Transporter | |
| OCTN2/SLC22A5 | Organic Cation/Carnitine | NM_003060 |
| | Transporter | |
| MATE1/SLC47A1 | Multidrug and Toxin Extrusion | NM_018242 |
| | 1 | |
| MATE2K/SLC47A2 | Multidrug and Toxin Extrusion | NM_001099646 |
| | 2K | |
| URAT1/SLC22A12 | Urate Transporter 1 | NM_144585 |
| ASBT/SLC10A2 | Apical Sodium/Bile Acid Co- | NM_000452 |
| | Transporter | |
| NTCP/SLC10A1 | Sodium/Taurocholate Co- | NM_003049 |
| | Transporting Peptide | |

*In this instance, OATP1B1 includes OATP1B1*1a and OATP1B1*1b, OATP1B1*5, and OATP1B1*15.

In exemplary, non-limiting embodiments, the one or more genes encoding a human SLC transporter include at least one of the genes set forth in Table 3.

TABLE 3

| | | GENE |
|---|---|---|
| | | ACCESSION |
| GENE NAME | FULL NAME | NUMBER |
| OATP1B1*1a/ | Organic Anion-Transporting | NM_006446.4 |
| SLCO1B1*1a | Polypeptide 1B1 Wild Type | |
| | (388A) | |
| OATP1B1*1b/ | Organic Anion-Transporting | RS2306283 |
| SLCO1B1*1b | Polypeptide 1B1 SNP 388A > G | |
| OATP1B1*5/ | Organic Anion-Transportin | RS4149056 |
| SLCO1B1*5 | Polypeptide 1B1 388A, SNP | |
| | 521T > C | |
| OATP1B1*15/ | Organic Anion-Transportin | RS2306283, |
| SLCO1B1*15 | Polypeptide 1B1 SNP 388A > G, | RS4149056 |
| | SNP 521T > C | |
| OATP1B3/ | Organic Anion-Transporting | NM_019844 |
| SLCO1B3 | Polypeptide 1B3 | |
| OAT1/SLC22A6 | Organic Anion Transporter 1 | NM_004790 |
| OAT3/SLC22A8 | Organic Anion Transporter 3 | NM_004254 |
| OCT1/SLC22A1 | Organic Cation Transporter 1 | NM_003057 |
| OCT2/SLC22A2 | Organic Cation Transporter 2 | NM_003058 |

In some particular embodiments, the recombinant cells include one or more transiently overexpressed genes selected from the group consisting of MDR1/Mdr1a/Mdr1b, MRP1/Mrp1, MRP2/Mrp2, MRP3/Mrp3, MRP4/Mrp4, MRP5/Mrp5, MRP6/Mrp6, MRP7/Mrp7, MRP 8/Mrp8, BCRP/Bcrp, BSEP/Bsep, OATP2/Oatp2, OATP1B3/Oatp1b3, OAT1/Oat1, OAT2/Oat2, OAT3/Oat3, OAT4/Oat4, OCT1/Oct1, OCT2/Oct2, OATP1/Oatp1, PEPT1/Pept1, PEPT2/Pept2, OCTN1/Octn1, OCTN2/Octn2, MATE1/Mate1, MATE2K/Mate2, URAT1/Urat1, ASBT/Asbt, NTCP/Ntcp, and a combination thereof. In other particular embodiments, the recombinant cells include one or more transiently overexpressed genes selected from the group consisting of OATP2/Oatp2, OATP1B3/Oatp1b3, OAT1/Oat1, OAT2/Oat2, OAT3/Oat3, OAT4/Oat4, OCT1/Oct1, OCT2/Oct2, OATP1/Oatp1, PEPT1/Pept1, PEPT2/Pept2, OCTN1/Octn1, OCTN2/Octn2, MATE1/Mate1, MATE2K/Mate2, URAT1/Urat1, ASBT/Asbt, NTCP/Ntcp, and a combination thereof. In some embodiments, OATP2/Oatp2 is selected from the group consisting of OATP1B1*1a, OATP1B1*1b, OATP1B1*5, OATP1B1*15 and combinations thereof. In some embodiments, OATP2/Oatp2 is OATP1B1*1b. In some embodiments, OATP2/Oatp2 is OATP1B1*5. In some embodiments, OATP2/Oatp2 is OATP1B1*15.

In some embodiments, the recombinant cells include one or more transiently overexpressed genes that encodes a solute carrier transporter protein selected from the group consisting of monkey Oatp1b1, monkey Oatp1b3, dog Oatp1b4, rat Oatp1b2, rat Oatp1a1, rat Oatp1a4, and a combination thereof. In some particular embodiments, the recombinant cells include one or more transiently overexpressed genes that encodes monkey Oatp1b1 and monkey Oatp1b3. In other particular embodiments, the recombinant cells include one or more transiently overexpressed genes that encondes dog Oatp1b4. In even further particular embodiments, the recombinant cells include one or more transiently overexpressed genes that encondes rat Oatp1b2.

In some embodiments, the recombinant cells include one or more transiently overexpressed genes encoding a drug metabolizing enzyme. In embodiments, the recombinant cells include one or more transiently overexpressed genes encoding a drug metabolizing enzyme selected from the group consisting of cytochrome drug metabolizing enzymes, non-cytochrome drug metabolizing enzymes, and a combination thereof. In particular embodiments, the recombinant cells include one or more transiently overexpressed genes encoding CYPs P450, UDP-glucouronyl transferases, alcohol dehydrogenases, monoamine oxidases, or aldehyde oxidases.

In embodiments, activity of the drug transporter protein, drug metabolizing enzyme, or combination thereof is detectable in a population of the recombinant cells prior to cryopreservation and/or following thaw from cryopreservation at an uptake ratio of at least 5. In some embodiments, activity of the drug transporter protein, drug metabolizing enzyme, or combination thereof is detectable in a population of the recombinant cells prior to cryopreservation at an uptake ratio of at least 5. In recombinant cells wherein activity of the drug transporter protein, drug metabolizing enzyme, or combination thereof is detectable in a population of the recombinant cells prior to cryopreservation, the recombinant cells have been transfected with one or more genes and have been cultured (such as, e.g., via suspension or adherent culture) for a period of time sufficient to initiate protein expression in the recombinant cells prior to cryopreservation.

In some embodiments, activity of the drug transporter protein, drug metabolizing enzyme, or combination thereof would be detectable in a population of the recombinant cells following thaw from cryopreservation at an uptake ratio of at least 5. In some particular embodiments, activity of the drug transporter protein, drug metabolizing enzyme, or combination thereof would be detectable in a population of the recombinant cells within four hours of thaw from cryopreservation at an uptake ratio of at least 5. In recombinant cells wherein activity of the drug transporter protein, drug metabolizing enzyme, or combination thereof would be detectable in a population of the recombinant cells within 4 hours of thaw from cryopreservation, the recombinant cells have been transfected with one or more genes and have been cultured (such as, e.g., via suspension or adherent culture) prior to cryopreservation for a period of time sufficient to initiate protein expression in the recombinant cells. In embodiments wherein activity of the drug transporter protein, the drug metabolizing enzyme, or combination thereof is detectable at an uptake ratio of at least 5 prior to cryopreservation and/or would be detectable at an uptake ratio of at least 5 within 4 hours of thaw from cryopreservation, the recombinant cells are assay-ready. In some particular embodiments, assay-ready recombinant cells are suitable for screening drug candidates (without culturing) to determine whether they have an effect on drug transporter proteins and/or drug metabolizing enzymes. For example, drug candidates can be screened to determine if any are substrates or inhibitors of the drug transporter proteins and/or drug metabolizing enzymes. In particular, if a drug candidate is a substrate of a drug transporter protein and/or a drug metabolizing enzyme, the drug candidate will be affected. For example, if the drug candidate is a substrate of a drug transporter protein, the drug candidate will be translocated in and/or out of the recombinant cell via the drug transporter protein. However, if the drug candidate is an inhibitor of the drug transporter protein, the drug candidate will inhibit translocation of a substrate of the drug transporter protein in and/or out of the recombinant cell. In some embodiments, screening is conducted using whole cells and/or subcellular fractions thereof (such as, e.g., via use of microsomes and/or cytosol).

In some embodiments, activity of the drug transporter protein, the drug metabolizing enzyme, or combination thereof would be detectable in a population of recombinant cells within 48 hours of thawing from cryopreservation. In some particular embodiments, activity of the drug transporter protein, the drug metabolizing enzyme, or combination thereof, would be detectable in a population of recombinant cells at about 0 hours post-thaw from cryopreservation (i.e., immediately following thaw from cryopreservation), at about 4 hours post-thaw from cyropreservation, at about 8 hours post-thaw from cryopreservation, at about 16 hours post-thaw from cryopreservation, at about 24 hours post-thaw from cryopreservation, or at about 48 hours post-thaw from cryopreservation. In such particular embodiments, the recombinant cells have been transfected with one or more genes prior to cryopreservation and have been cultured for a period of time sufficient to initiate protein expression in the recombinant cells either prior to cryopreservation or following thaw from cryopreservation.

In some particular embodiments, the recombinant cells include one or more transiently overexpressed genes encoding a drug transporter protein, wherein activity of the drug transporter protein is detectable in a population of recombinant cells prior to cryopreservation at an uptake ratio of at least 5. In embodiments, activity of the drug transporter protein is detectable in a population of recombinant cells prior to cryopreservation at an uptake ratio of from about 5 to about 150. In some embodiments, the recombinant cells include one or more transiently overexpressed genes encoding a drug transporter protein, wherein activity thereof is detectable in a population of recombinant cells prior to cryopreservation at an uptake ratio of from about 5 to about 150, or from about 10 to about 250, or from about 25 to about 100, or about 30. In embodiments, the population of recombinant cells is selected from the group consisting of an adherent population (such as, e.g., a plated population), a suspended population, or a combination thereof.

In some particular embodiments, the recombinant cells include one or more transiently overexpressed genes encoding a drug transporter protein, wherein activity of the drug transporter protein would be detectable in a population of recombinant cells following thaw from cryopreservation at an uptake ratio of at least 5. In embodiments, activity of the drug transporter protein would be detectable in a population of recombinant cells following thaw from cryopreservation at an uptake ratio of from about 5 to about 150. In some embodiments, the recombinant cells include one or more transiently overexpressed genes encoding a drug transporter protein, wherein activity thereof would be detectable in a population of recombinant cells following thaw from cryopreservation at an uptake ratio of from about 5 to about 150, or from about 10 to about 250, or from about 25 to about 100, or about 30. In embodiments, the population of recombinant cells is selected from the group consisting of an adherent population (such as, e.g., a plated population), a suspended population, or a combination thereof.

Methods for detecting activity of a drug transporter protein and/or drug metabolizing enzyme in recombinant cells are known to the skilled artisan, such as, e.g., via uptake assay. In exemplary, non-limiting embodiments, activity of a drug transporter protein and/or drug metabolizing enzyme is detected by washing the cells with appropriate buffer (such as, e.g., pre-warmed HBSS buffer with Ca²⁺ and Mg²⁺ for thawed Corning® TransportoCells™) and pre-incubating the cells in appropriate buffer (such as, e.g., HBSS buffer for 10 minutes at 37° C. for thawed Corning® TransportoCells™). An uptake assay may then be performed by adding appropriate labeled substrates (such as, e.g., radio-labeled substrates) and/or appropriate labeled inhibitors (such as, e.g., radio-labeled inhibitors) and incubating at 37° C. for an appropriate period of time (such as, e.g., 2 minutes for MATE1/2K; 5 minutes for OATP1B1*1a, OATP1B3, and OAT1/3; or 10 minutes for OCT1/2). Reactions may be stopped by removing substrate solutions and washing the cells with cold buffer (such as, e.g., HBSS buffer for Corning® TransportoCells™). Cells may be lysed with M-Per Mammalian Protein extraction reagent and uptake activity may be quantified using liquid scintillation counting normalized for protein concentration in each sample. Kinetic parameters may be determined via non-linear regression using SigmaPlot. For each substrate concentration, the initial uptake may be calculated by subtracting the initial rate determined in control cells from that obtained in experimental, recombinant cells expressing the drug transporter protein and/or drug metabolizing enzyme. IC50 values may be determined using Sigmoidal Hill four-parameter equation. Activity of a drug transporter protein and/or drug metabolizing enzyme may be detected via an adherent assay (such as, e.g., a plated population) or a suspension assay, as described subsequently.

Embodiments of recombinant cells including one or more transiently overexpressed genes encoding a drug transporter protein, a drug metabolizing enzyme, or combination thereof have been described in detail. Reference will now be made in detail to embodiments of processes of preparing cryopreserved, transiently transfected recombinant cells.

### II. Processes of Preparing Cryopreserved, Transiently Transfected Recombinant Cells

In embodiments, processes of preparing cryopreserved transiently transfected recombinant cells are disclosed. The processes may include transiently transfecting cells with one or more genes encoding a drug transporter protein, a drug metabolizing enzyme, or combination thereof (providing transiently transfected recombinant cells), and cryopreserving the transiently transfected recombinant cells within 72 hours of transfection. In some embodiments, the processes include transiently transfecting cells with one or more genes encoding a drug transporter protein, a drug metabolizing enzyme, or combination thereof (providing transiently transfected recombinant cells), and cryopreserving the transiently transfected recombinant cells within 48 hours of transfection. In embodiments, a population of the transiently transfected recombinant cells transiently overexpress the one or more genes encoding the drug transporter protein, drug metabolizing enzyme, or combination thereof at a detectable level prior to cryopreservation and/or following thaw from cryopreservation. In some embodiments, the transiently transfected recombinant cells transiently overexpress the one or more genes encoding the drug transporter protein, drug metabolizing enzyme, or combination thereof at a detectable level prior to cryopreservation and/or following thaw from cryopreservation, wherein the detectable level is an uptake ratio of at least 5 (i.e., 5:1). In some particular embodiments, the transiently transfected recombinant cells transiently overexpress the one or more genes encoding the drug transporter protein, drug metabolizing enzyme, or combination thereof at a detectable level prior to cryopreservation, wherein the detectable level is an uptake ratio of at least 5.

In embodiments, the recombinant cells are as previously described with regard to recombinant cells. In embodiments, the cells are transiently transfected with one or more genes encoding a drug transporter protein, a drug metabolizing enzyme, or a combination thereof. In embodiments, the one or more genes encoding a drug transporter protein, drug metabolizing enzyme, or a combination thereof are as previously described with regard to recombinant cells.

In some embodiments, the cells are transiently transfected with one or more genes encoding a drug transporter protein to provide transiently transfected recombinant cells. In embodiments, transfection includes introducing genes into a population of cells. Gene delivery systems (e.g., transient transfection systems) for introducing one or more genes into a population of cells are known to a skilled artisan. Exemplary, non-limiting transient transfection systems include virus-based gene delivery methods, lipid-based transfection methods, electroporation (i.e., EP), and combinations thereof. With regard to virus-based gene delivery methods, such methods require special handling due to safety concerns. With regard to lipid-based transfection methods, such methods are costly and are not amenable to large-scale manufacturing processes. Additionally, lipid-based transfection methods provide relatively low gene delivery efficiency and relatively delayed protein expression (e.g., from 72 hours to 96 hours post-transfection) (data not shown). With regard to EP, EP is amenable to large-scale manufacturing processes and avoids the safety issues of viral-based gene delivery methods. Further, EP results in relatively efficient gene delivery. As demonstrated by the data disclosed herein, EP leads to the surprising and unexpected effect of improved (decreased) lot-to-lot variability, improved manufacturability of the instantly-disclosed transiently transfected, cryopreserved cells, as well as an improved, earlier response time and increased levels of expression and activity of transiently transfected drug transporter proteins as compared to lipid-based transfection methods. As such, in embodiments, the processes of preparing transiently transfected recombinant cells include transiently transfecting cells via EP. In exemplary, non-limiting embodiments, cells are pelleted down via centrifugation, aspirated, and resuspended in appropriate EP buffer (such as, e.g., buffer available from MaxCyte, Cat. No. B201). A cell stock may then be prepared by transferring the cell suspension to 50 ml Falcon tubes, pelleting down via centrifugation, and resuspending in appropriate EP buffer to a final cell density of, e.g., 100×10⁶ cells/ml. DNA to be used for EP may then be prepared in sterile water (such as, e.g., to a final concentration of 5 mg/ml). For each sample, 0.4 ml of the cell stock and DNA may be transferred to a sterile 1.5 ml eppendorf tube and processed in an OC-400 Processing Assembly (available from MaxCyte, Cat. No. OC-400R) for EP. Vectors used for transient transfection utillize the CMV promoter (such as, e.g., pCMV6-XL5, pCMV6-Entry, and pCMV6-AC vectors available from Origene).

After gene delivery into a population of cells, gene(s) encoding a drug transporter protein and/or a drug metabolizing enzyme will be overexpressed such that activity of the protein(s) encoded therefrom are detectable following thaw from cryopreservation. Drug candidates can be tested to determine if any are substrates or inhibitors of the protein(s) encoded from the overexpressed gene(s) by incubation of the recombinant cells therewith. In particular, if a drug candidate is a substrate of a drug transporter protein and/or a drug metabolizing enzyme, the drug candidate will be affected. For instance, if the drug candidate is a substrate of a drug transporter protein, the drug candidate will be translocated in or out of the recombinant cell via the drug transporter protein. However, if the drug candidate is an inhibitor of the drug transporter protein, the drug candidate will inhibit translocation of a substrate of the drug transporter protein in or out of the recombinant cell.

Additionally, in embodiments, the recombinant cells of the present disclosure are further transfected with RNAi and/or siRNA of the transiently overexpressed genes to knockdown and/or knockout the expression thereof. For example, primary cells (such as, e.g., hepatocytes) may be transfected with RNAi and/or siRNA directed against any ABC transporters, SLC transporters, and/or any drug metabolizing enzymes to knockdown and/or knockout the expression thereof.

In embodiments, the transiently transfected recombinant cells are cryopreserved within 72 hours of transfection. In embodiments wherein a population of cells which overexpress the one or more genes at a detectable level prior to cryopreservation is desired, the transiently transfected recombinant cells are cultured for a period of time sufficient to initiate protein expression in the recombinant cells prior to cryopreservation. In some embodiments, the transiently transfected recombinant cells are cultured for from about 24 hours to about 72 hours, or for about 48 hours prior to cryopreservation. In embodiments wherein a population of cells which would overexpress the one or more genes at a detectable level within 4 hours following thaw from cryopreservation is desired, the transiently transfected recombinant cells are cultured for a period of time sufficient to initiate protein expression in the recombinant cells prior to cryopreservation. In some embodiments, the transiently transfected recombinant cells are cultured for from about 24 hours to about 72 hours, or for about 48 hours prior to cryopreservation. In embodiments wherein a population of cells which would overexpress the one or more genes at a detectable level within 48 hours following thaw from cryopreservation is desired (e.g., at about 0 hours post-thaw from cryopreservation (i.e., immediately following thaw from cryopreservation), at about 1 hour post-thw from cyropreservation, at about 4 hours post-thaw from cyropreservation, at about 8 hours post-thaw from cryopreservation, at about 16 hours post-thaw from cryopreservation, at about 24 hours post-thaw from cryopreservation, or at about 48 hours post-thaw from cryopreservation), the transiently transfected recombinant cells are cultured for from about 24 hours to about 72 hours, or for about 48 hours prior to cryopreservation.

In embodiments, the transiently transfected recombinant cells are cultured in suitable culturing conditions via suspension culture or adherent culture (such as, e.g., a plated culture). In some embodiments, the transiently transfected recombinant cells are cultured in suitable culturing conditions via suspension culture in shaker flasks. In other embodiments, the transiently transfected recombinant cells are cultured in suitable culturing conditions via adherent culture in microplates or T-flasks. In embodiments, the transiently transfected recombinant cells are cultured in suitable culturing conditions via suspension or adherent culture at a cell density of from about 100K cells/well to 300K cells/well. In specific embodiments, the transiently transfected cells are cultured in suitable culturing conditions via suspension culture or adherent culture at a cell density of at least about 200K cells/well. In other specific embodiments, the transiently transfected cells are cultured in the presence of sodium butyrate. In further specific embodiments, the transiently transfected cells are cultured in the presence of sodium butyrate provided to a final concentration of 5 mM. In embodiments, the transiently transfected recombinant cells are harvested following culturing. Methods for harvesting transiently transfected recombinant cells are known to the skilled artisan, such as, e.g., via centrifugation and/or treatment with Trypsin or Dulbecco's Phosphate-Buffered Saline.

In embodiments, the transiently transfected recombinant cells are cryopreserved within 72 hours of transfection. In some embodiments, the transiently transfected recombinant cells are cryopreserved within 48 hours of transfection. Methods for cryopreserving transiently transfected recombinant cells are known to the skilled artisan. In exemplary, non-limiting embodiments, transiently transfected recombinant cells are cryopreserved by pelleting down transiently transfected recombinant cells via centrifugation and resuspending in freshly prepared appropriate ice-cold freezing media (such as, e.g., 9 parts culturing medium and 1 part DMSO). Then, cryo vials may be filled with 1-2 ml of the suspended transiently transfected recombinant cells and placed on ice-cold Mr. Frosty freezing container and stored in a −80° C. freezer overnight.

In embodiments, a population of the transiently transfected recombinant cells transiently overexpress the one or more genes encoding the drug transporter protein, drug metabolizing enzyme, or combination thereof at a detectable level prior to cryopreservation and/or following thaw from cryopreservation, wherein the detectable level is an uptake ratio of at least 5. In some embodiments, a population of the transiently transfected recombinant cells transiently overexpress the one or more genes encoding the drug transporter protein, drug metabolizing enzyme, or combination thereof at a detectable level prior to cryopreservation, wherein the detectable level is an uptake ratio of at least 5.

In some embodiments, activity of the drug transporter protein, drug metabolizing enzyme, or combination thereof would be detectable in a population of the recombinant cells following thaw from cryopreservation at an uptake ratio of at least 5. In some particular embodiments, activity of the drug transporter protein, drug metabolizing enzyme, or combination thereof would be detectable in a population of the recombinant cells within four hours of thaw from cryopreservation at an uptake ratio of at least 5. In embodiments wherein activity of the drug transporter protein, the drug metabolizing enzyme, or combination thereof is detectable at an uptake ratio of at least 5 prior to cryopreservation and/or would be detectable at an uptake ratio of at least 5 within 4 hours of thaw from cryopreservation, the recombinant cells are assay-ready. In some particular embodiments, assay-ready recombinant cells are suitable for screening drug candidates (such as, e.g., without culturing) to determine whether they have an effect on drug transporter proteins and/or drug metabolizing enzymes.

In particular embodiments, a suspended population of the transiently transfected recombinant cells would overexpress the one or more genes encoding the drug transporter protein, the drug metabolizing enzyme, or the combination thereof, at a detectable level at about hours post-thaw from cryopreservation (i.e., immediately following thaw from cryopreservation). In particular embodiments, a suspended population of the transiently transfected recombinant cells would overexpress the one or more genes encoding the drug transporter protein, the drug metabolizing enzyme, or the combination thereof, at a detectable level within 1 hour post thaw from cryopreservation. For example, in some embodiments, the transiently transfected recombinant cells would overexpress the one or more genes encoding the drug transporter protein, the drug metabolizing enzyme, or the combination thereof, at the detectable level within 1 hour post thaw from cryopreservation as determined via a suspension assay. In other particular embodiments, an adherent population (such as, e.g., a plated population) of the transiently transfected recombinant cells would overexpress the one or more genes encoding the drug transporter protein, the drug metabolizing enzyme, or the combination thereof, at a detectable level within 4 hours post thaw from cryopreservation. For example, in some embodiments, the transiently transfected recombinant cells would overexpress the one or more genes encoding the drug transporter protein, the drug metabolizing enzyme, or the combination thereof, at the detectable level within 4 hours post thaw from cryopreservation as determined via an adherent (such as, e.g., a plated) assay.

In some embodiments, a population of the transiently transfected recombinant cells would transiently overexpress the one or more genes encoding the drug transporter protein, the drug metabolizing enzyme, or combination thereof at a detectable level following thaw from cryopreservation, wherein the detectable level is an uptake ratio of at least 5. In some particular embodiments, a population of the transiently transfected recombinant cells would transiently overexpress the one or more genes encoding the drug transporter protein, the drug metabolizing enzyme, or combination thereof at a detectable level at about 0 hrs post-thaw from cyropreservation (i.e., immediately post-thaw), at about 1 hour post-thaw from cryopreservation, at about 4 hours post-thaw from cryopreservation, at about 8 hours post-thaw from cryopreservation, at about 16 hours post-thaw from cryopreservation, at about 24 hours post-thaw from cryopreservation, or at about 48 hours post-thaw from cryopreservation.

In some particular embodiments, the transiently transfected recombinant cells transiently overexpress one or more genes encoding a drug transporter protein, wherein activity of the drug transporter protein is detectable in a population of recombinant cells prior to cryopreservation at an uptake ratio of at least 5. In embodiments, the detectable level is at an uptake ratio of from about 5 to about 150. In some embodiments, the detectable level is at an uptake ratio of from about 5 to about 150, or from about 10 to about 250, or from about 25 to about 100, or about 30. In embodiments, the population of recombinant cells is selected from the group consisting of an adherent population (such as, e.g., a plated population), a suspended population, or a combination thereof.

In some particular embodiments, the transiently transfected recombinant cells would transiently overexpress one or more genes encoding a drug transporter protein, wherein activity of the drug transporter protein would be detectable in a population of recombinant cells following thaw from cryopreservation at an uptake ratio of at least 5. In embodiments, the detectable level is an uptake ratio of from about 5 to about 150. In some embodiments, the detectable level is at an uptake ratio of from about 5 to about 150, or from about 10 to about 250, or from about 25 to about 100, or about 30. In embodiments, the population of recombinant cells is selected from the group consisting of an adherent population (such as, e.g., a plated population), a suspended population, or a combination thereof.

Methods for detecting activity of a drug transporter protein and/or drug metabolizing enzyme in recombinant cells are as previously described with regard to recombinant cells. In exemplary, non-limiting embodiments, activity of the drug transporter protein and/or drug metabolizing enzyme may be detected via an uptake assay.

Embodiments of processes of preparing cryopreserved, transiently transfected recombinant cells have been described in detail. Reference will now be made in detail to embodiments of suspension assays with specific reference to FIG. 21.

III. Suspension Assays for Assessing Activity of Drug Transporter Proteins and/or Drug Metabolizing Enzymes in Recombinant Cells

In embodiments, suspension assays for assessing activity of drug transporter proteins and/or drug metabolizing enzymes in recombinant cells are disclosed. Referencing FIG. 21, in some embodiments, the suspension assays include: (1) providing suspended, recombinant cells transiently transfected with one or more genes encoding a drug transporter protein, a drug metabolizing enzyme, or a combination thereof, with a substrate; (2) stopping reaction of the drug transporter protein, drug metabolizing enzyme, or combination thereof, with the substrate; (3) separating the recombinant cells and the substrate via centrifugation; and (4) assessing activity of the drug transporter protein, drug metabolizing enzyme, or combination thereof.

In embodiments, the recombinant cells are as previously described with regard to recombinant cells. In embodiments, the cells are transiently transfected with one or more genes encoding a drug transporter protein, a drug metabolizing enzyme, or a combination thereof, as previously described with regard to processes of preparing cryopreserved, transiently transfected recombinant cells. In embodiments, the one or more genes encoding a drug transporter protein, drug metabolizing enzyme, or a combination thereof, are as previously described with regard to recombinant cells.

In embodiments, suspended, recombinant transiently transfected cells are provided and/or contacted with a substrate. In some embodiments, the recombinant, transiently transfected cells are suspended in buffer (such as, e.g., Hank's Balanced Salt Solution with Ca²⁺ and Mg²⁺). In some particular embodiments, the recombinant, transiently transfected cells are suspended in buffer to a cell density of about 1×10⁶ cells/ml.

In embodiments, suspended, recombinant cells transiently transfected with one or more genes encoding a drug transporter protein and/or a drug metabolizing enzyme are provided and/or contacted with a substrate. In some embodiments, the substrate is provided in a substrate solution. In some embodiments, suspended, recombinant cells transiently transfected with one or more genes encoding a drug transporter protein are provided and/or contacted with a substrate solution. In some embodiments, the substrate solution includes a substrate upon which the drug transporter protein is capable of acting and/or a buffer. In some particular embodiments, the substrate solution contains a labeled substrate (such as, e.g., a radio-labeled or fluorescently-labeled substrate) upon which the drug transporter protein is capable of acting and/or a buffer. For example, in embodiments wherein suspended, recombinant cells are transiently transfected with one or more genes encoding Organic Anion-Transporter Polypeptide 1B1, the substrate solution may contain Estradiol 17-β Glucuronide, fluorescein methotrexate, 8-fluorescein-cAMP, and/or Hank's Balanced Salt Solution. In some particular embodiments, the suspended, recombinant transiently transfected cells are provided at a cell density of about 200K cells/well and about 50 μL of the 5× substrate solution is provided for a final 1× substrate. Both cells and substrate are resuspened/dissolved in buffer. In embodiments, the suspended, transiently transfected recombinant cells are provided and/or contacted with a substrate solution in a vessel, such as, e.g., a microplate.

In embodiments, a biochemical reaction of the drug transporter protein and/or drug metabolizing enzyme and substrate is inhibited and/or stopped. In some embodiments, biochemical reaction of the drug transporter protein and substrate is inhibited and/or stopped. In particular embodiments, the biochemical reaction is inhibited and/or stopped by providing and/or contacting the substrate with cold buffer. In some particular embodiments, the cold buffer is Hank's Balanced Salt Solution. In further particular embodiments, about 50 μl of Hank's Balanced Salt Solution is provided. In some other embodiments, reaction of the drug transporter protein and substrate is inhibited and/or stopped by providing and/or contacting the substrate with cold buffer and placing the suspended, transiently transfected recombinant cells and/or substrate on ice. In some particular embodiments, placing the suspended, transiently transfected recombinant cells and/or substrate on ice involves placing a vessel (such as, e.g., a microplate) including the transiently transfected recombinant cells and/or substrate on ice.

In embodiments, the suspended, transiently transfected recombinant cells and/or substrate are separated via centrifugation. In some embodiments, the suspended, transiently transfected recombinant cells and/or substrate are centrifuged at about 1000 g for about 1 minute at about 4° C. Upon centrifugation, a cell pellet including the transiently transfected recombinant cells may form. In some embodiments, a cell pellet formed during centrifugation is washed with buffer. In some particular embodiments, the wash buffer is Hank's Balanced Salt Solution. In some further particular embodiments, the cell pellet formed during centrifugation is washed 3 times with Hank's Balanced Salt Solution (HBSS).

In embodiments, activity of the drug transporter protein, drug metabolizing enzyme, or combination thereof is assessed. In some embodiments, methods for assessing and/or detecting the activity of the drug transporter protein, the drug metabolizing enzyme, or combination thereof are as previously described with regard to recombinant cells. In exemplary, non-limiting embodiments, activity of the drug transporter protein and/or drug metabolizing enzyme may be assessed via lysing and the appropriate radiolabel and/or fluorescent analysis of the radiolabled or fluorescent substrate.

Embodiments of suspension assays have been described in detail.

It should now be understood that various aspects of recombinant cells, preparation processes, and suspension assays are described herein and that such aspects may be utilized in conjunction with various other aspects.

In a first aspect, the disclosure provides a recombinant cell including one or more transiently overexpressed genes encoding a drug transporter protein. The recombinant cell is cryopreserved, and activity of the drug transporter protein is detectable in a population of the recombinant cells prior to cyropreservation at an uptake ratio of at least 5.

In a second aspect, the disclosure provides a recombinant cell of the first aspect, in which the activity of the drug transporter protein would be detectable in a population of the recombinant cells following thaw from cryopreservation at an uptake ratio of at least 5.

In a third aspect, the disclosure provides a recombinant cell of the first or the second aspect, in which the activity of the drug transporter protein would be detectable in the population of the recombinant cells following thaw from cryopreservation at an uptake ratio of from about 5 to about 150.

In a fourth aspect, the disclosure provides a recombinant cell of the first to the third aspects, in which the activity of the drug transporter protein would be detectable in a plated population of the recombinant cells following thaw from cryopreservation at an uptake ratio of from about 5 to about 30 within 4 hours of thawing.

In a fifth aspect, the disclosure provides a recombinant cell of the first to the third aspects, in which the activity of the drug transporter protein would be detectable in a suspended population of the recombinant cells following thaw from cryopreservation at an uptake ratio of from about 5 to about 150 within 1 hour of thawing.

In a sixth aspect, the disclosure provides a recombinant cell of the first to the fifth aspects, in which the drug transporter protein is selected from the group consisting of an ATP binding cassette transporter and a solute carrier transporter protein.

In a seventh aspect, the disclosure provides a recombinant cell of the first to the sixth aspects, in which the one or more transiently overexpressed genes is selected from the group consisting of MDR1/Mdr1a/Mdr1b, MRP1/Mrp1, MRP2/Mrp2, MRP3/Mrp3, MRP4/Mrp4, MRP5/Mrp5, MRP6/Mrp6, MRP7/Mrp7, MRP 8/Mrp8, BCRP/Bcrp, BSEP/Bsep, OATP2/Oatp2, OATP1B3/Oatp1b3, OAT1/Oat1, OAT2/Oat2, OAT3/Oat3, OAT4/Oat4, OCT1/Oct1, OCT2/Oct2, OATP1/Oatp1, PEPT1/Pept1, PEPT2/Pept2, OCTN1/Octn1, OCTN2/Octn2, MATE1/Mate1, MATE2K/Mate2, URAT1/Urat1, ASBT/Asbt, NTCP/Ntcp, and a combination thereof.

In an eighth aspect, the disclosure provides a recombinant cell of the first to the seventh aspects the one or more transiently overexpressed genes is selected from the group consisting of OATP2/Oatp2, OATP1B3/Oatp1b3, OAT1/Oat1, OAT2/Oat2, OAT3/Oat3, OAT4/Oat4, OCT1/Oct1, OCT2/Oct2, OATP1/Oatp1, PEPT1/Pept1, PEPT2/Pept2, OCTN1/Octn1, OCTN2/Octn2, MATE1/Mate1, MATE2K/Mate2, URAT1/Urat1, ASBT/Asbt, NTCP/Ntcp, and a combination thereof.

In a ninth aspect, the disclosure provides a recombinant cell of the seventh to the eighth aspects, in which OATP2/Oatp2 is selected from the group consisting of OATP1B1*1a, OATP1B1*1b, OATP1B1*5, OATP1B1*15 and combinations thereof.

In a tenth aspect, the disclosure provides a recombinant cell of the eighth aspect, in which OATP2/Oatp2 is OATP1B1*1a.

In an eleventh aspect, the disclosure provides a recombinant cell of the eighth aspect, in which OATP2/Oatp2 is OATP1B1*1b.

In a twelfth aspect, the disclosure provides a recombinant cell of the eighth aspect, in which OATP2/Oatp2 is OATP1B1*5.

In a thirteenth aspect, the disclosure provides a recombinant cell of the eighth aspect, in which OATP2/Oatp2 is OATP1B1*15.

In a fourteenth aspect, the disclosure provides a recombinant cell of the first to the thirteenth aspects, in which the one or more transiently overexpressed genes is derived individually from a human or an animal species selected from the group consisting of a mouse, a rat, a guinea pig, a dog, and a monkey.

In a fifteenth aspect, the disclosure provides a recombinant cell of the first to the fourteenth aspect, in which the one or more genes encodes a solute carrier transporter protein selected from the group consisting of monkety Oatp1a1, monkey Oatp1b3, dog Oatp1b4, rat Oatp1b2, rat Oatp1a1, rat Oatp1a4, and combinations thereof.

In a sixteenth aspect, the disclosure provides a recombinant cell of the first to the fifteenth aspects, in which the cell is a hepatocyte.

In a seventeenth aspect, the disclosure provides a recombinant cell of the first to the fifteenth aspects, in which the cell is an endothelial cell.

In a eighteenth aspect, the disclosure provides a process of preparing cryopreserved transiently transfected recombinant cells, the process including: transiently transfecting cells with one or more genes encoding a drug transporter protein to provide the transiently transfected recombinant cells, and cryopreserving the transiently transfected recombinant cells within 48 hours of transfection, wherein a population of the transiently transfected recombinant cells transiently overexpress the one or more genes encoding the drug transporter protein at a detectable level prior to cryopreserving the transiently transfected recombinant cells, and wherein the detectable level prior to cryopreserving is an uptake ratio of at least 5.

In a nineteenth aspect, the disclosure provides a process according to the eighteenth aspect, in which transient transfection of the cells includes electroporation.

In a twentieth aspect, the disclosure provides a process according to the eighteenth or the nineteenth aspects, in which the transiently transfected recombinant cells are cryopreserved at about 24 hours to about 48 hours post transfection.

In a twenty-first aspect, the disclosure provides a process according to any of the eighteenth to the twentieth aspects, in which a population of the transiently transfected recombinant cells would overexpress the one or more genes encoding the drug transporter protein at the detectable level following thaw from cryopreservation, and the detectable level following thaw from cryopreservation is an uptake ratio of at least 5.

In a twenty-second aspect, the disclosure provides a process according to any of the eighteenth to the twenty-first aspects, in which a suspended population of the transiently transfected recombinant cells would overexpress the one or more genes encoding the drug transporter protein at the detectable level following thaw from cryopreservation within 1 hour post thaw.

In a twenty-third aspect, the disclosure provides a process according to any of the eighteenth to the twenty-second aspects, in which the detectable level following thaw from cryopreservation is an uptake ratio of from about 5 to about 150.

In a twenty-fourth aspect, the disclosure provides a process according to any of the eighteenth to the twenty-third aspects, in which a plated population of the transiently transfected recombinant cells would overexpress the one or more genes encoding the drug transporter protein at the detectable level following thaw from cryopreservation within 4 hours post thaw.

In a twenty-fifth aspect, the disclosure provides a process according to any of the eighteenth to the twenty-fourth aspects, in which the detectable level following thaw from cryopreservation is an uptake ratio of from about 5 to about 30.

### EXAMPLES

Cells were cultured under standard sterile practices for cell culture, and transiently transfected using EP. Following EP, cells were assayed for protein activity both before as well as after being frozen, thawed and plated. As detailed below, cells cultured in suspension and adherent cell cultures were both successfully transiently transfected and exhibited substantial activity of the recombinant protein following thaw from cryopreservation.

### Example 1

### Development and Characterization of Transiently Transfected Recombinant Cells Expressing a Gene Encoding a Drug Transporter Protein

Cells Cultured in Suspension—Experimental Protocol.

In brief, on Day 1, FreeStyle 293 Cells (hereinafter, “FS293”) and 293-F cells were each passaged into appropriate sized shaker flasks at a density of 0.7-1.0×10⁶ cell/ml using supplemented CD293 medium (hereinafter, “CD293 medium”; available from Gibco, Cat. No. 11913-019, Life Technologies Corp., Carlsbad, Calif., supplemented with 4 mM L-Glutamine; available from Gibco, Cat. No. 25030-081, Life Technologies Corp.) or supplemented Excell™ 293 serum free media (available from Sigma, Cat. No. 14571C, Sigma-Aldrich, St. Louis, Mo.) supplemented with 6 mM L-Glutamine. Cell viability and cell number were determined using a Cellometer (available from Nexcelom Bioscience, Lawrence, Mass.).

On Day 2, EP of cells was executed. In short, following a determination of cell viability and cell density, cells were pelleted down by spinning at 100 g for 5 min, after which the media was aspirated and cells resuspended in 30 ml EP Buffer (available from MaxCyte, Cat. No. B201, MaxCyte Inc., Gaithersburg, Md.). The cell suspension was transferred to 50 ml Falcon tubes, pelleted down as described above, and resuspended in an appropriate amount of EP Buffer to reach 100×10⁶ cells/ml which was used as the cell stock. DNAs to be used for EP were prepared in sterile water at a final concentration of 5 mg/ml. For each sample, 0.4 ml of cell stock and DNA was placed in a sterile 1.5 ml eppendorf tube resulting in a final concentration of 200 μg/ml (see Table 4) or 300 μg/ml DNA (see Tables 10 and 11) and cell density of 40×10⁶ cells per sample.

TABLE 4

| | | | | CELL | |
|---|---|---|---|---|---|
| | | | | STOCK | |
| | | CELL | | VOL. | [DNA] |
| | SAMPLE # | TYPE | PLASMID(S) | (ml) | (ug/ml) |
| A | 1, 2 | FS293 | pOATP1B1 | 0.4 | 200 |
| B | 3, 4 | | pCMV6 | 0.4 | 200 |
| C | 5 | | EP Buffer (16 μl) | 0.4 | — |
| D | 6, 7 | 293-F | pOATP1B1 | 0.4 | 200 |
| E | 8, 9 | | pCMV6 | 0.4 | 200 |
| F | 10 | | EP Buffer (16 μl) | 0.4 | — |

Samples were transferred into an OC-400 Processing Assembly (available from MaxCyte, Cat. No. OC-400R, MaxCyte Inc.) which followed the manufacturer's instructions for EP of HEK cells. Following EP, the cells were carefully pipetted out and transferred into the bottom of a 125 ml shaker flask and incubated for 20 min at 37° C. with 8% CO₂, after which pre-warmed 40 ml CD293 media was added into the shaker flask to reach cell density at 1×10⁶ cells/ml. The cells were incubated for 30 min at 37° C. and 8% CO₂. After 30 min recovery, cell viability and cell density were determined. A portion of cells (i.e., 20×10⁶ cells) was used for plating and the rest was cryopreserved, or all of the cells were cryopreserved. It is contemplated that recombinant cells may be cryopreserved within 48 hrs of transfection and exhibit activity of protein(s) encoded from transfected gene(s) at a detectable level following thaw from cryopreservation.

For plating cells following EP, 20×10⁶ cells were pelleted down by spinning at 100 g for 5 min and then resuspended in 20 ml pre-warmed CD293 media (cell density of 1×10⁶ cells/ml). Cells were placed in 24-well tissue culture plates poly-D-Lysine coated, Corning Biocoat™ (available from Corning Life Sciences, Tewksbury, Mass.) at a density of 0.2×10⁶ cells/well and 0.4×10⁶ cells/well and incubated at 37° C. with 8% CO₂ so as to determine the impact of seeding density on uptake activity. Media was replaced 4 hours later and then every 24 hours until the day of assaying. On Day 4, cells were assayed for OATP1B1 activity as described below.

For cryopreservation, cells were pelleted then resuspended in freshly prepared ice-cold freezing media (9 parts supplemented CD293 medium and 1 part DMSO which was syringe filtered to sterilize) at a density of 10×10⁶ cell/ml. Cryo vials were filled with 1 ml of this cell suspension, and placed on ice-cold Mr. Frosty freezing container (available from Thermal Scientific), which was stored in −80° C. freezer overnight after which the vials were transferred into liquid nitrogen.

Cryopreserved cells were assayed for OATP1B1 activity as described below. In brief, on Day 1, cryopreserved cells were removed from liquid nitrogen to dry ice, and then thawed in a water bath at 37° C. for about 2 min. Cells were transferred into 10 ml of supplemented DMEM media (DMEM with high glucose (available from Gibco, Cat. No. 11965092, Life Technologies Corp.), supplemented with 0.1 mM non-essential amino acids (available from Gibco, Cat. No. 11140050, Life Technologies Corp.), 10% FBS (available from SAFC Biosciences, Cat. No. 12016C, Sigma)) prewarmed to a temperature of about 37° C. and the viability and cell density determined. Cells were pelleted down and resuspended in supplemented DMEM media at a cell density of 1×10⁶ viable cells/ml. Cells were plated in the same manner described above for plating cells following EP (which had not been cryopreserved) and assayed for OATP1B1 activity at 24, 48 and 72 hrs following plating thereof.

Adherent Cell Cultures—Experimental Protocol.

In brief, HEK293 cells were cultured in 5 Layer Corning® CellStack® (available from Corning Inc. Life Sciences, Lowell, Mass.) using plating media containing DMEM (high glucose) available from Gibco Cat. No. 11965118, Life Technologies Corp.; Penicillin-Streptomycin (10,000 units/ml) available from Gibco Cat. No. 15140-122, Life Technologies Corp.; L-Glutamine (200 mM) available from Gibco Cat. No. 25030-081, Life Technologies Corp.; Sodium Pyruvate, available from Gibco Cat. No. 11360, Life Technologies Corp.; FBS available from Sigma-Aldrich Corp. in a ratio of 100:1:1:1:10. On Day 1, about 24 hrs before EP, HEK293 cells were trypsinized, cell viability and cell number determined after which cells were passaged to fresh multilayer chamber flasks at 30-40% confluency. Cells were incubated at 37° C. with 5% CO₂.

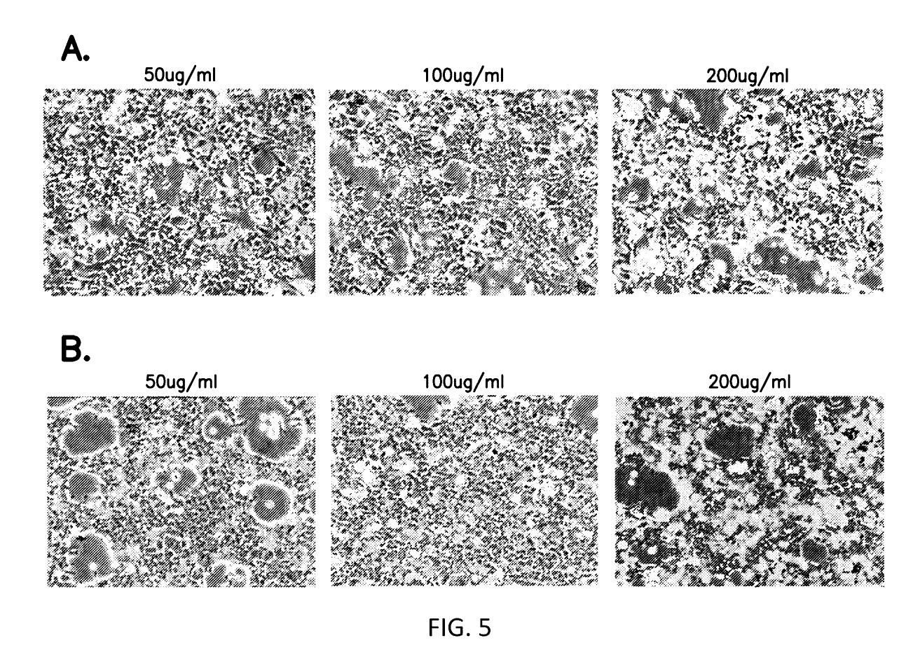

On Day 2, EP of cells was executed. In short, cells were harvested, cell viability and cell number determined after which cells were pelleted down by spinning at 100 g for 5 min and the media aspirated. Cells were resuspended in EP buffer and pelleted down by spinning at 100 g for 5 min, then resuspended in an appropriate amount of EP Buffer to reach 50×10⁶ cells/ml which was used as the cell stock. DNAs to be used for EP were prepared in sterile water at a final concentration of 5 mg/ml. For each sample used for OC-400 processing assembly, 0.4 ml of cell stock and DNA was placed in a sterile 1.5 ml eppendorf tube resulting in a final concentration of 50 μg/ml, 100 μg/ml, 200 μg/ml or 400 μg/ml DNA as indicated in FIGS. 5-9 and cell density of 40×10⁶ cells per sample. For each sample used for CL-2 processing assembly, 10 ml of cell stock and DNA was placed in 50 ml sterile conical tube resulting in a final concentration of 100 μg/ml DNA.

Samples were transferred into an OC-400 or CL-2 processing assembly (available from MaxCyte, Cat. No. OC-400R and CL2-R, MaxCyte Inc.) which followed the manufacture instructions for EP of HEK cells. Following EP, the cells were carefully pipetted out and transferred into 6-well tissue culture plates and incubated for 20 min at 37° C. with 5% CO₂, after which cells were removed and placed in a 50 ml conical tube containing pre-warmed plating media. Cell viability and cell density were determined. A portion of cells (i.e., 20×10⁶ cells) was used for plating and the rest was cryopreserved.

For plating cells following EP, cells were pelleted down by spinning at 100 g for 5 min and then resuspended in pre-warmed plating media (cell density of 1×10⁶ cells/ml). Cells were placed in 24-well tissue culture plates (poly-D-Lysine coated, Corning Biocoat™ (available from Corning Life Sciences) at a density of 0.4×10⁶ cells/well and incubated at 37° C. with 5% CO₂. Media was replaced 4 hours later and then every 24 hours until the day of assaying. On Days 4 and 6, cells were assayed for OATP1B1 activity.

For cryopreservation, cells were pelleted then resuspended in freshly prepared ice-cold freezing media (9 parts plating medium and 1 part DMSO which was syringe filtered to sterilize) at a density of 10×10⁶ cell/ml. Cryo vials were filled with 1 ml of this cell suspension, and placed on ice-cold Mr. Frosty freezing container (available from Thermal Scientific) stored in −80° C. freezer overnight after which the vials were stored in liquid nitrogen.

Cryopreserved cells were assayed for OATP1B1 activity. Notably, cells were plated in the same manner described above for plating cells following EP (which had not been cryopreserved) and assayed for OATP1B1 activity (as described below) at 48 hrs following plating thereof.

Assaying Transporter Activity—Experimental Protocol and Results.

In brief, substrate solution was prepared for OATP1B1*1a and OATP1B1*1b using 2 μM estradiol-17β-glucuronide (99% of cold E17βG and 1% of [³H]-E17βG); for OATP1B3 using 2 μM CCK-8 (99% of cold CCK-8 and 1% of [³H]-CCK-8); for OAT1 short using 1 μM Para-aminohippurate (PAH) (90% of cold PAH and 10% of [³H]-PAH); for OAT1 long using 1 μM or 3 μM Para-aminohipurate (PAH) (90% of cold PAH and 10% of [³H]-PAH); for OAT3 using 1 μM or 2 μM Estrone-3-sulfate (99% of cold E3S and 1% of [³H]-E3S), for OCT1 and OCT2 using 30 μM GTetraethylammonium Bromide (100% [¹⁴C]-TEA); MATE1 and MATE2K using 10 μM Metformin (100% [¹⁴C]-Metformin) or 10 μM Tetraethylammonium Bromide (100% [¹⁴C]-TEA); in Krebs-Henseleit Buffer pH 7.4 (available from Sigma, Cat. No. K3753, Sigma-Aldrich) and incubated at 37° C. for at least 20 min. Culture media was aspirated from cells to be assayed and cells washed thrice with pre-warmed KHB Buffer. Cells were subsequently incubated with Uptake Buffer at 37° C. for 10 min. For MATE1 and MATE2K, cells were washed and pre-incubated with KHB buffer containing 20 mM NH₄Cl for 10 min. Assays were initiated by adding 0.3 ml substrate solution into each well and incubated at 37° C. for 5 min with samples for OCT1 and OCT2 incubated for 10 min.

The reaction was quickly stopped after the incubation period by aspirating substrate solution from cells then washing cells thrice with cold Uptake Buffer. Cells were then incubated with lysing solution (M-per mammalian protein extraction reagent) for 15-20 minutes while being shaken. The substrate solution was triturated and 0.4 ml of the resultant cell lysis placed in 5 ml scintillation tube with 5 ml of scintillation liquid for analysis with scintillation counter.

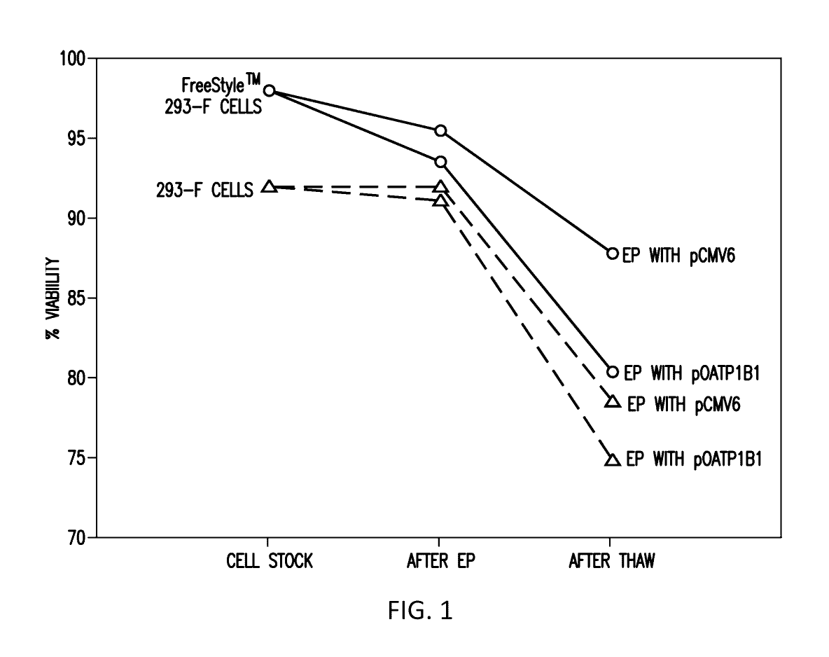

As illustrated in FIG. 1, cell viability dropped 1-5% after EP relative to that of the cell stock. Additionally, after cryopreservation, cell viability dropped an additional 10-15% relative to that after EP. Nonetheless, even after EP and thaw from cryopreservation, cell viability is greater than 75%.

Cell morphology and uptake activity was examined following cryopreservation after 30 min recovery and 24 hours recover post-transfection. Table 5 illustrated cell morphology and uptake activity with 24 hours recovery was reduced compared to 30 min recovery.

TABLE 5

| | | Cell | | |
|---|---|---|---|---|
| | Recovery time prior | Confluency | Uptake Activity | |
| SAMPLE | to cryopreservation | at 24 hrs | (pmole/mg/min) | S:N |
| OATP1B1 | 24 HOURS | 40-50% | 0.59 | 0.44 |
| OATP1B1 | 30 MIN | 70-75% | 5.78 | 4.32 |
| VECTOR | 30 MIN | 90-95% | 1.34 | |

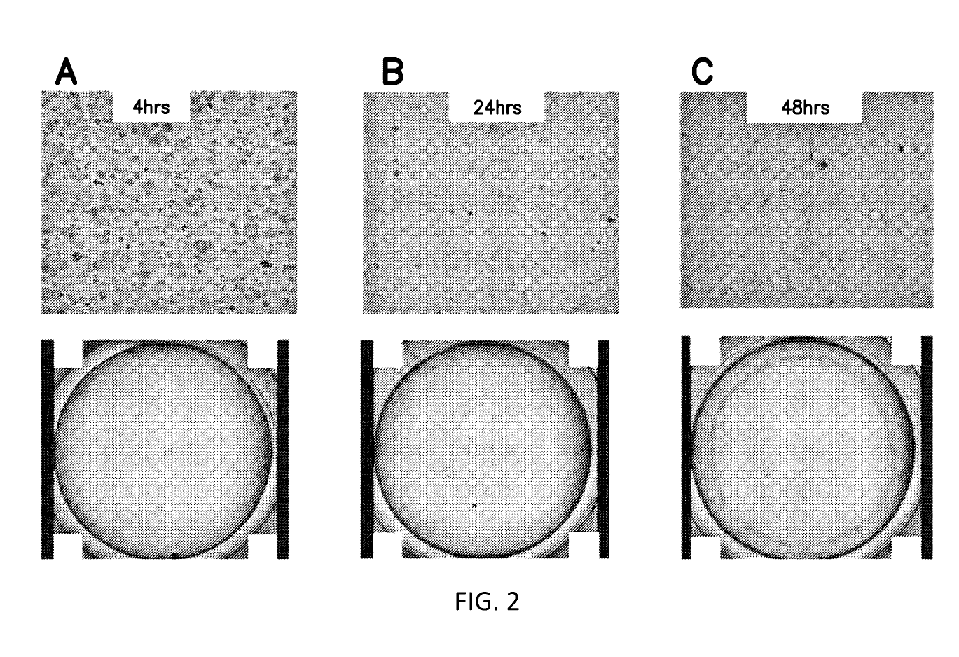

Cell morphology and confluency of transfected cells thawed from cryopreservation were examined after various periods of time following plating at a density of 0.4×10⁶ cells per well in 24-well poly-D-lysine coated Corning Biocoat™ plates. In particular, FIG. 2 illustrates OATP1B1 transiently transfected cells cultured at 4 hrs, 24 hrs and 72 hrs post plating. Additionally, cell confluency at 24 hrs, 48 hrs and 72 hrs post-plating of these cells is recorded in Table 6 below.

TABLE 6

| CELLS | 24 hrs | 48 hrs | 72 hrs |
|---|---|---|---|
| FS293 with pOATP1B1 | 80-90% | 90-95% | 80-85% |
| FS293 with pCMV6 vector | 70-80% | 90-95% | 90% |
| 293-F with pOATP1B1 | 90-95% | 95-100% | 80-85% |
| 293-F with pCMV6 vector | 90-95% | 95-100% | 80-85% |

Desirably, after EP and cryopreservation, the cells form a monolayer on poly-D-lysine coated Corning Biocoat™ plates achieving 80-90% confluency at 24 hrs post-plating, 90%400% confluency at 48 hrs post-plating.

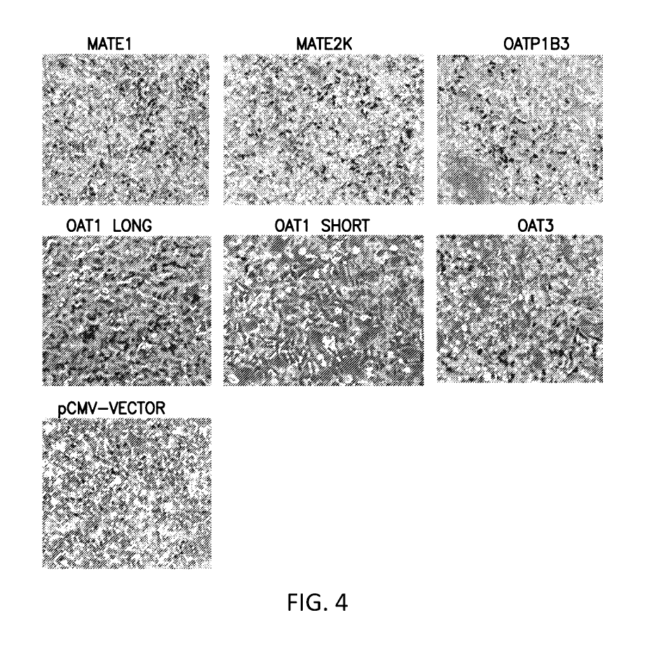

FIG. 4 illustrates cells, transiently transfected with MATE1, MATE2K, OATP1B3, OAT1 long, OAT1 short, OAT3, and pCMV vector respectively, cultured at 24 hrs post plating after thawed from cryopreservation.

As illustrated in FIG. 5, the expression of Green Fluorescent Protein (GFP) in adhesion HEK293 cells was increased with increasing concentration of DNA. Additionally, GFP expression increased at the 48 hr timepoint relative to the 24 hr timepoint. In particular, GFP transfection efficiency by EP achieved 100% at 24 hrs with 200 μg/ml DNA and 100% fluorescent cell staining at 48 hrs with 100 μg/ml DNA. Hence, GFP protein expression level in transfected cells increased with increased DNA concentration and at 48 hrs relative to 24 hrs.

Uptake activity of suspension cultured 293 cells transfected with OATP1B1 (pOATP1B1) and control vector (pCMV) were assayed at various time points following EP. In brief, transfected cells were plated at a density of 0.4×10⁶ cells/well in 24-well poly-D-lysine coated Corning Biocoat™ plates following EP or after thaw from cryopreservation. OATP1B1 uptake activity and uptake ratio were determined using probe substrate, estradiol-17β-glucuronide, in both fresh plated cells (“fresh”) and cryopreserved cells (“cryo”) at various timepoints post plating as detailed in Table 7 below.

TABLE 7

| CELLS/CULTURE | | UPTAKE ACTIVITY | | |
|---|---|---|---|---|
| MEDIA, FRESH OR | CELL PLATING | (pmol/mg/min)/confluence | | UPTAKE |
| CRYO | TIME POINT (HR) | pOATP1B1 | pCMV | RATIO |
| 293-F in CD293, fresh | 48 | 15.4 (85%) | 0.7 (90%) | 22.0 |
| 293-F in CD293, cryo | 48 | 15.1 (95-100%) | 0.9 (95-100%) | 16.8 |
| FS293 in Excell, cryo | 24 | 36.4 | 1.9 | 19.2 |
| | 48 | 10.0 | 0.7 | 14.3 |
| | 72 | 6.6 | 1.0 | 6.6 |
| 293-F in CD293, cryo | 24 | 27.4 | 1.5 | 18.3 |
| | 48 | 15.1 | 0.9 | 16.8 |
| | 72 | 9.9 | 1.0 | 9.9 |

Note:
The number appearing in parentheses is the cell confluency at assay time.

OATP1B1 uptake activity and uptake ratio in transfected cells following thaw from cryopreservation is consistent with those in freshly plated transfected cells. In both cells types 293-F and FS293, the highest uptake activity and uptake ratio is observed at 24 hrs post plating.

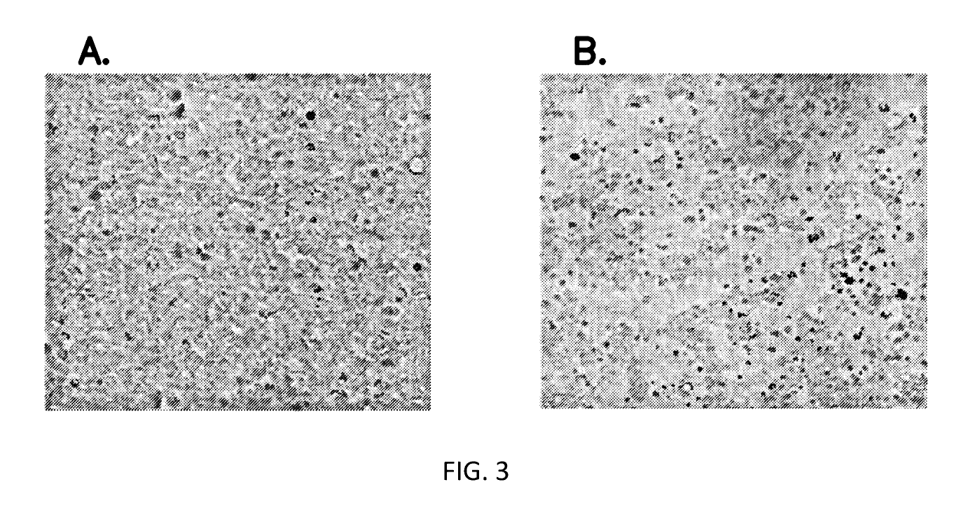

Morphology and cell confluency of transfected cells (i.e., FS293 or 293-F) were examined at 24 hrs, 48 hrs and 72 hrs post-plating in 24-well poly-D-lysine coated Corning Biocoat™ plates at plating density of either 0.4×10⁶ cells/well or 0.2×10⁶ cells/well after thaw from cryopreservation. Cell confluency at 24 hrs post-plating are summarized below in Table 8. Cell confluency at 48 hrs and 72 hrs are similar to those achieved at 24 hrs (data not shown). Additionally, FIG. 3 provides images of transfected cells plated at (A) 0.4×10⁶ cells per well and (B) 0.2×10⁶ cells per well 24 hrs post-plating following thaw from cryopreservation at a confluence of 90-95% and 60-70%, respectively.

TABLE 8

| CELLS, CULTURE MEDIA, | 0.4 × 10⁶ | 0.2 × 10⁶ |
|---|---|---|
| TRANSFECTED DNA | CELLS/WELL | CELLS/WELL |
| FS293 with pOATP1B1 in Excell | 80-90% | 30-50% |
| FS293 with pCMV vector in Excell | 70-80% | 50% |
| 293-F with pOATP1B1 in CD293 | 90-95% | 60-70% |
| 293-F with pCMV6 vector in CD293 | 90-95% | 80% |

For optimal assay performance, plating cells at a density of 0.4×10⁶ is preferable to that of 0.2×10⁶ as it achieves higher cell confluency and higher uptake activity.

TABLE 9

| | UPTAKE ACTIVITY | | |
|---|---|---|---|
| | (pmol/mg/min)/ | | |
| | confluence | | UPTAKE |
| CELLS | pOATP1B1 | pCMV6 | RATIO |
| FS293 cells, 0.2 × 10⁶ cells/well | 10.5 | 3.0 | 3.5 |
| FS293 cells, 0.4 × 10⁶ cells/well | 36.4 | 1.9 | 19.2 |
| 293-F cells, 0.2 × 10⁶ cells/well | 20.2 | 1.5 | 13.5 |
| 293-F cells, 0.4 × 10⁶ cells/well | 27.4 | 1.5 | 18.3 |

Following EP, cell viability was examined using trypan blue and hemocytometer or cellometer.

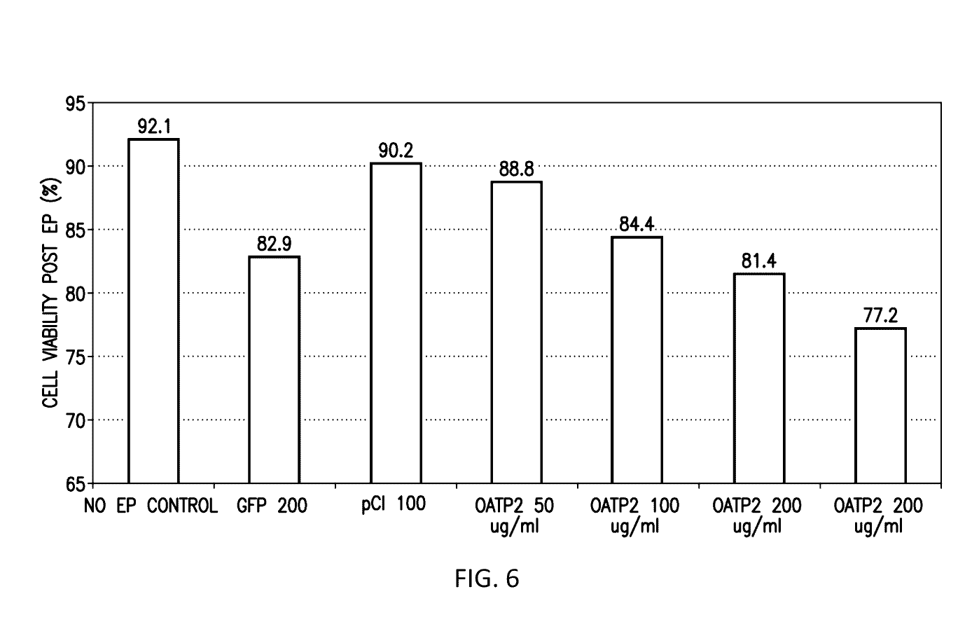

As illustrated in FIG. 6, when using adhesion HEK293 cells, cell viability post EP dropped with increasing amounts of DNA used in EP. Nonetheless, cell viability following transfection with pOATP1B1 was ranged from 89% to 77% and that following transfection with empty vector was 90%.

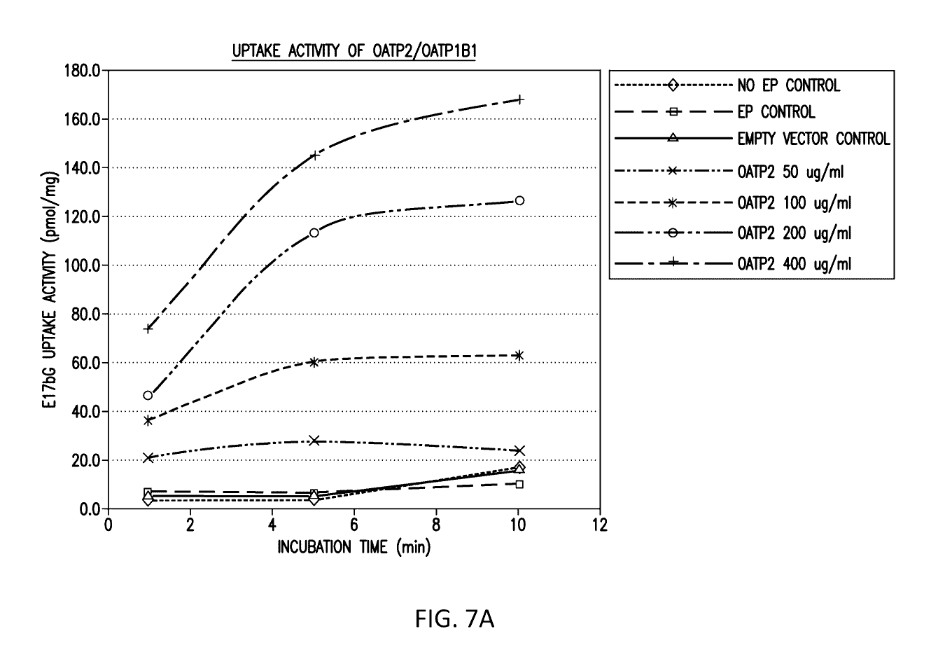

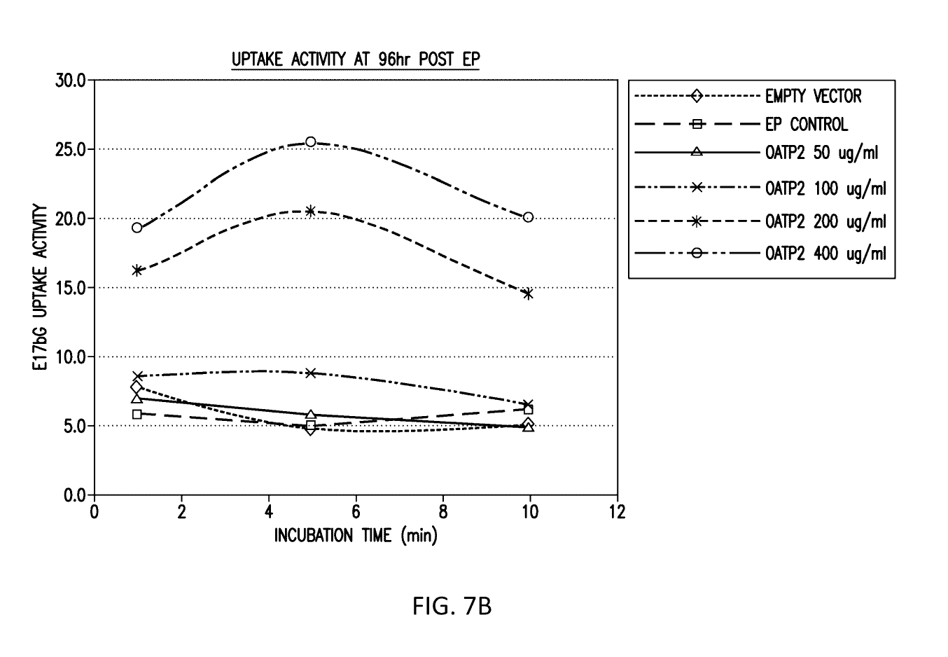

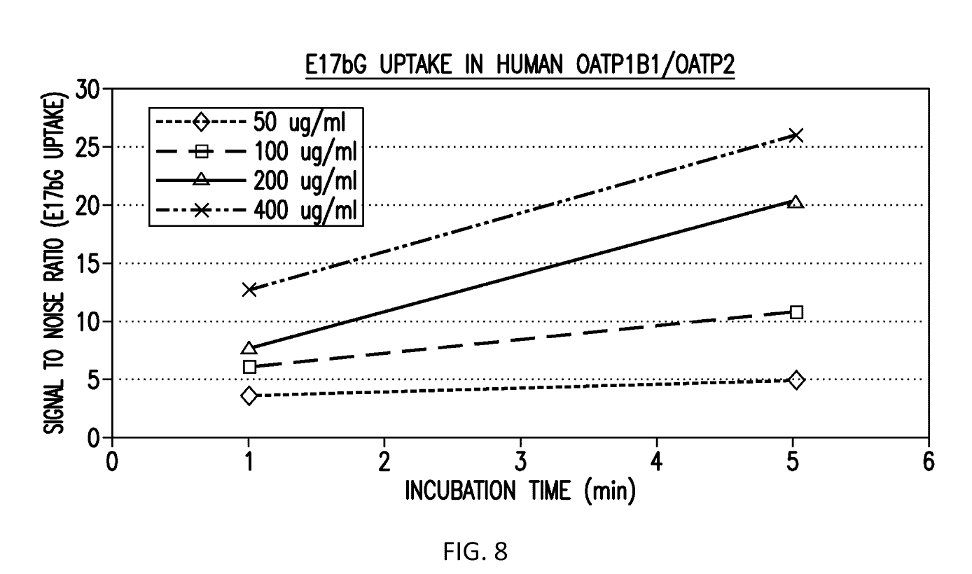

As illustrated in FIGS. 7A-7B, when using adhesion HEK293 cells, OATP1B1 mediated uptake of Estradiol-17β-glucuronide in the fresh plated transient transfected adhesion HEK293 cells is time-dependent. Notably, uptake activity and uptake ratio increased with increasing amounts of DNA used in EP. However, OATP1B1 mediated uptake of Estradiol-17β-glucuronide reduced at the 96 hr timepoint relative to the 48 hr timepoint. Further, as illustrated in FIG. 8, the signal to noise ratio (i.e., uptake ratio) of estradiol-17β-glucuronide increased with the increase of amount of DNA and assay incubation time, in adhesion HEK293 cells transfected with OATP1B1 relative to empty vector at 48 hrs post EP.

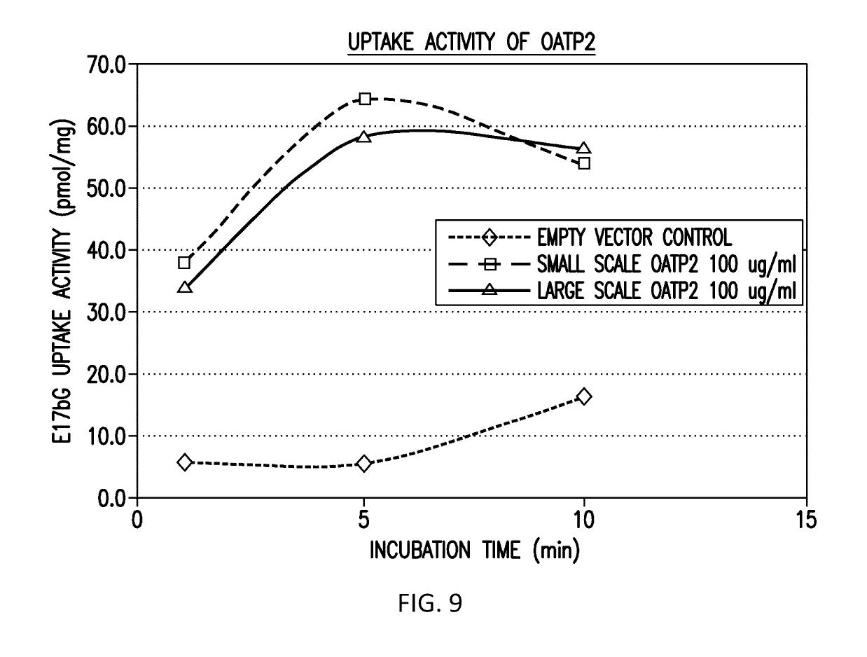

As illustrated in FIG. 9, when using adhesion HEK293 cells, estradiol-17β-glucuronide uptake in OATP1B1 transiently expressed HEK293 cells using small scale EP device and large scale EP device is consistent for both uptake activity and signal to noise ratio (i.e., uptake ratio). 100 μg/ml DNA was used in the experiments.

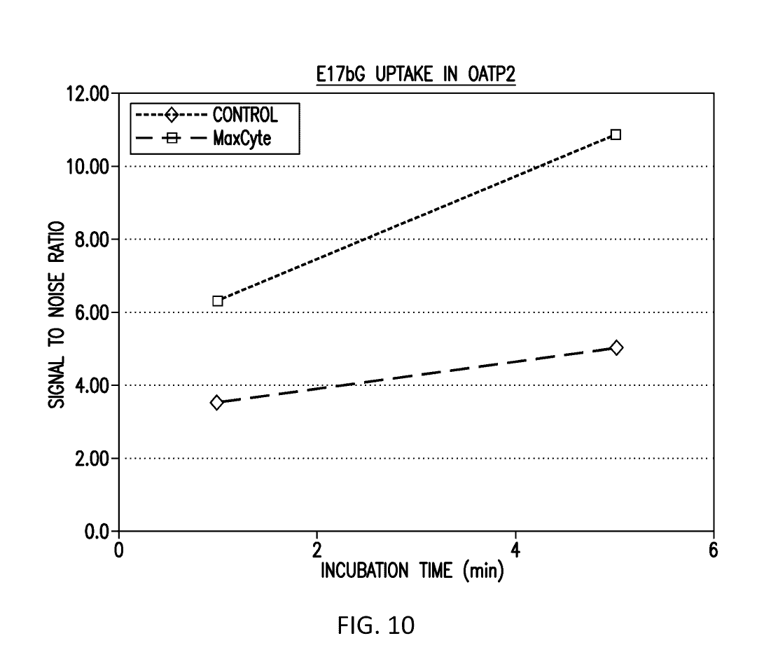

As illustrated in FIG. 10, when using adhesion HEK293 cells, OATP1B1 uptake activity is compared between the cells transfected using traditional lipid transfection reagent (control: lipofectamine 2000, available from Invitrogen) and EP using STX, MaxCyte Inc., Gaithersburg, Md. Notably, cells transfected using EP resulted in a pronouncedly greater signal to noise ratio relative to those cells transfected with lipid transfection reagent.

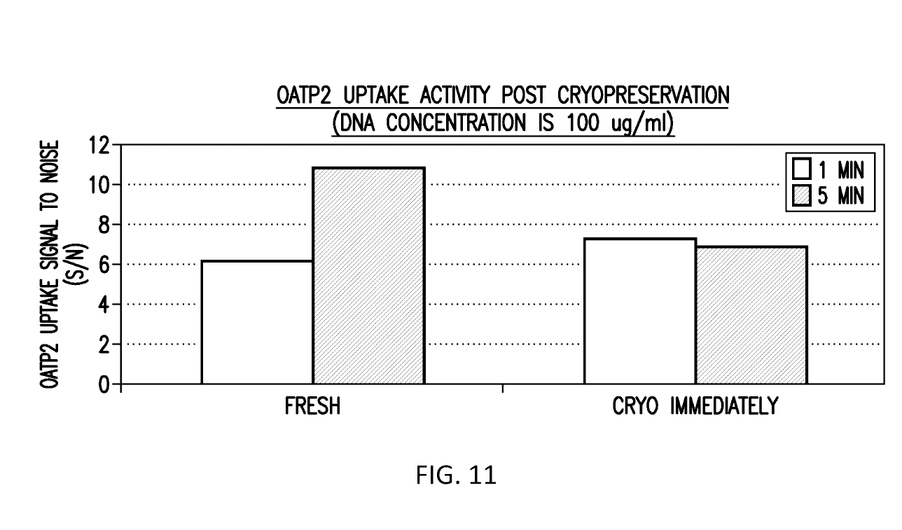

As illustrated in FIG. 11, when using adhesion HEK293 cells, OATP1B1 uptake activity in both freshly plated EP transfected cells and cells following thaw from cryopreservation was detectable.

Uptake activity of suspension cultured 293 cells transfected with OATP1B1*1a, OATP1B1*1b, OATP1B3, OAT1 long, OAT1 short, OAT3, OCT1, OCT2, MATE1, MATE2K or control vector (pCMV) were assayed at 24 hrs post plating after thaw from cryopreservation. In brief, the transfected cells were plated at a density of 0.4×10⁶ cells/well in 24-well poly-D-lysine coated Corning Biocoat™ plates following EP and after thaw from cryopreservation. SLC transporter uptake activity and uptake ratio were determined using probe substrates as indicated at 24 hrs post plating as detailed in Table 10 below.

TABLE 10

| | | UPTAKE | | |
|---|---|---|---|---|
| | | ACTIVITY | | |
| | | (pmol/mg/min)/ | | |
| | | SLC | | UPTAKE |
| TRANSPORTERS | SUBSTRATE | transporter | pCMV6 | RATIO |
| OATP1B1*1a | 2 μM E17bG | 41.0 | 1.03 | 40 |
| OATP1B1*1b | 2 μM E17bG | 32.6 | 0.88 | 37 |
| OATP1B3 | 2 μM CCK-8 | 28.7 | 0.2 | 145 |
| OATP1B3 | 2 μM CCK-8 | 77.0 | 0.79 | 98 |
| OAT1 long | 1 μM PAH | 13.1 | 0.3 | 39 |
| OAT1 short | 1 μM PAH | 9.7 | 0.3 | 29 |
| OAT1long | 3 μM PAH | 15.0 | 0.71 | 21 |
| OAT3 | 1 μM E3S | 44.7 | 1.2 | 38 |
| OAT3 | 2 μM E3S | 60.9 | 1.62 | 38 |
| OCT1 | 30 μM TEA | 127.6 | 5.63 | 23 |
| OCT2 | 30 μM TEA | 100.5 | 5.53 | 18 |
| MATE1 | 10 μM Metformin | 71.4 | 6.0 | 12 |
| | 10 μM TEA | 46.3 | 4.3 | 11 |
| MATE2K | 10 μM Metformin | 33.5 | 5.2 | 6.5 |
| | 10 μM TEA | 46.6 | 6.1 | 7.6 |

As reflected in Table 10 above, the recombinant cells exhibited strong uptake activity towards their specific prototypical substrate each of which had an uptake ratio above 10. Notably, an uptake ratio above 5 indicates a successful process.

As reflected in Table 11, the post-thaw viability for recombinant cryopreserved cells was determined to be above 90%.

TABLE 11

| Cells | Post-Thaw Viability |
|---|---|
| OATP1B1*1a | 94.2% |
| OATP1B1*1b | 96.1% |
| OATP1B3 | 95.5% |
| OAT1 long | 93.5% |
| OAT3 | 93.8% |
| OCT1 | 95.1% |
| OCT2 | 96.1% |

Each of these recombinant cells as well as a control vector (pCMV) was examined 24 hrs post-plating (after cryopreservation). Confluency for each of these cells 24 hrs post-plating was 85% or greater as reflected in Table 12 below.

TABLE 12

| Transfected Cells | 24-h confluency |
|---|---|
| OATP1B1*1a | 90% |
| OATP1B1*1b | 95% |
| OATP1B3 | 95% |
| OAT1 long | 90% |
| OAT3 | 90% |
| Vector | 95% |
| OCT1 | 95% |
| OCT2 | 85% |

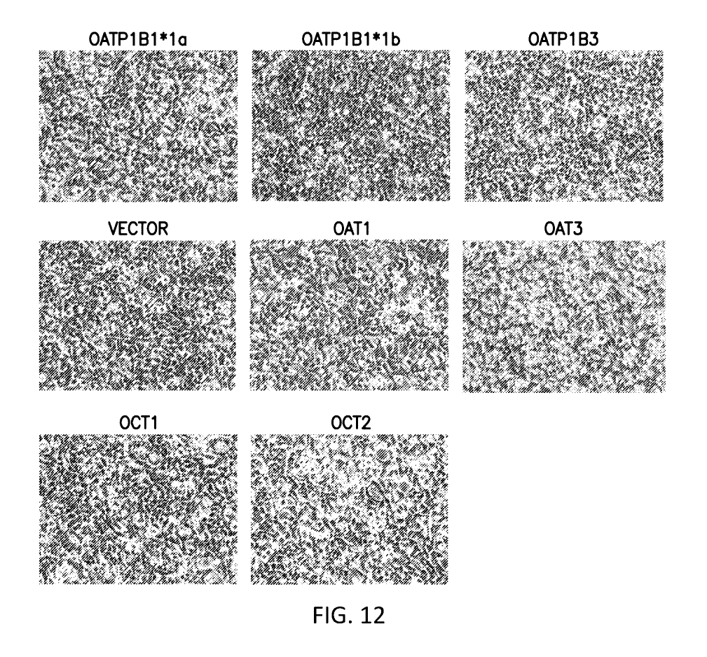

As illustrated in FIG. 12, each of the 8 cryopreserved recombinant cells formed a confluent monolayer following thawing, plating on Poly-D-Lysine plates and incubation for 24-hrs post-plating.

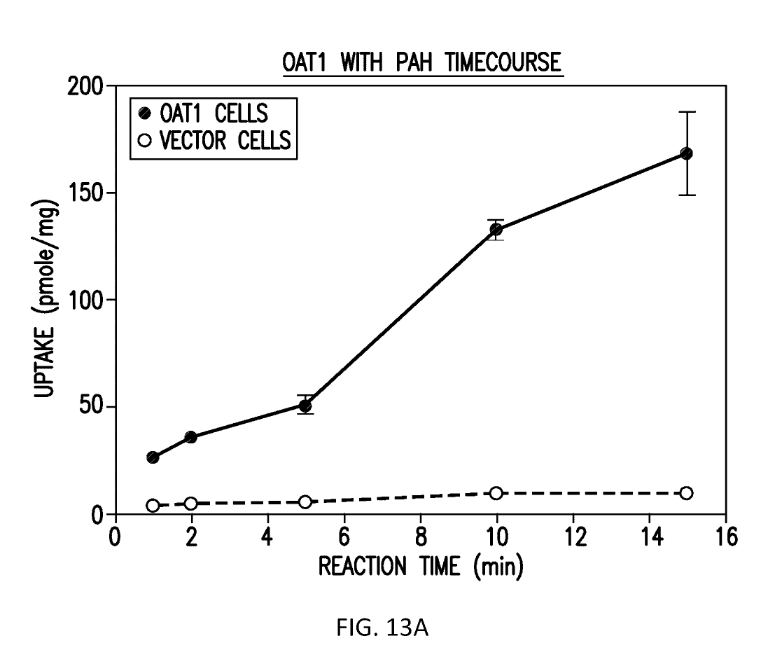

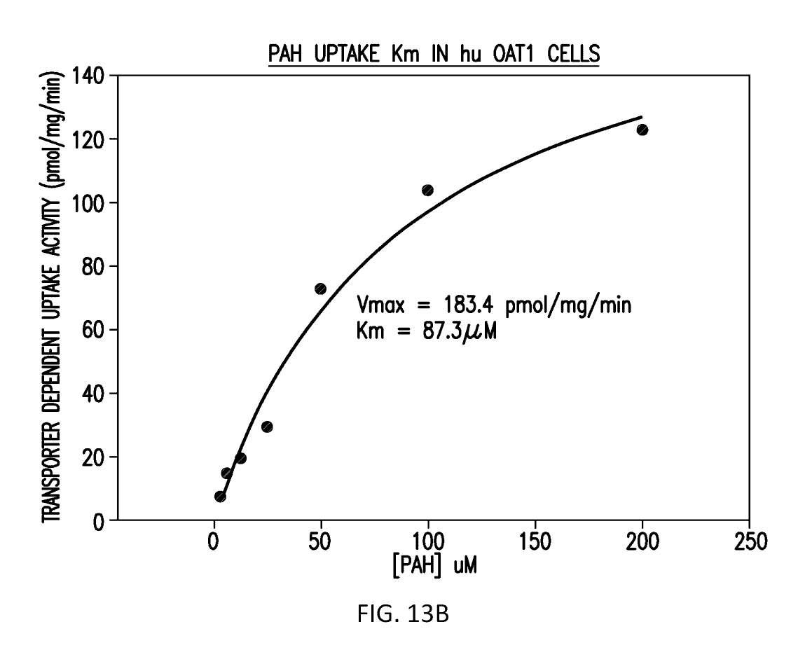

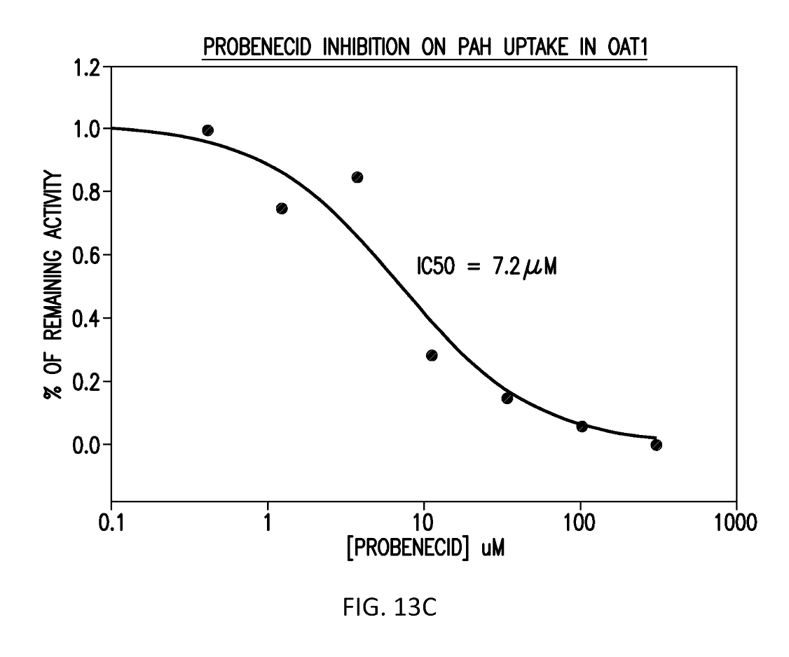

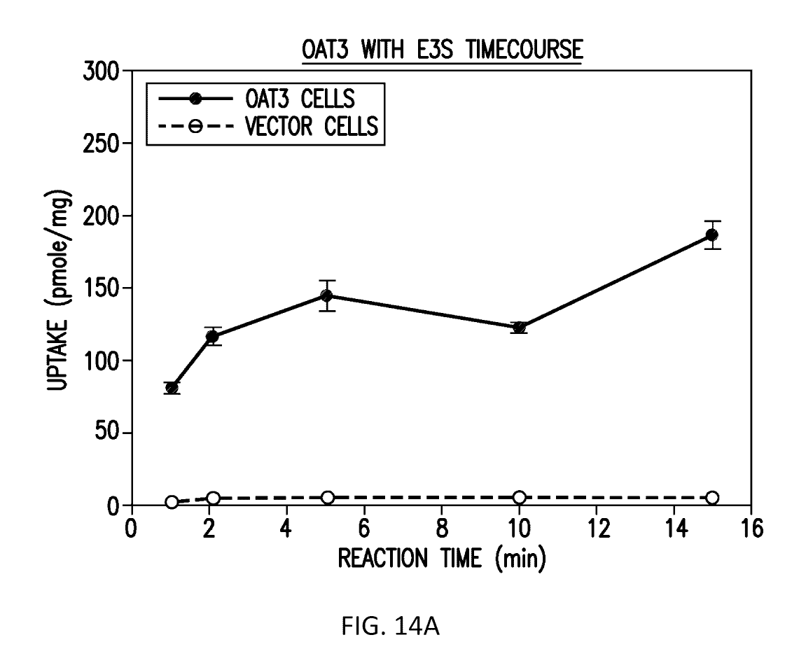

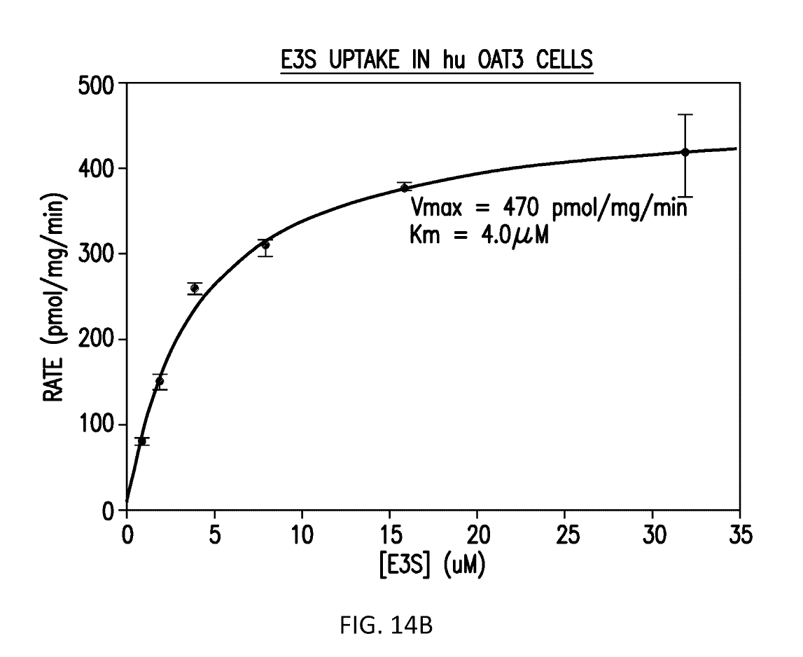

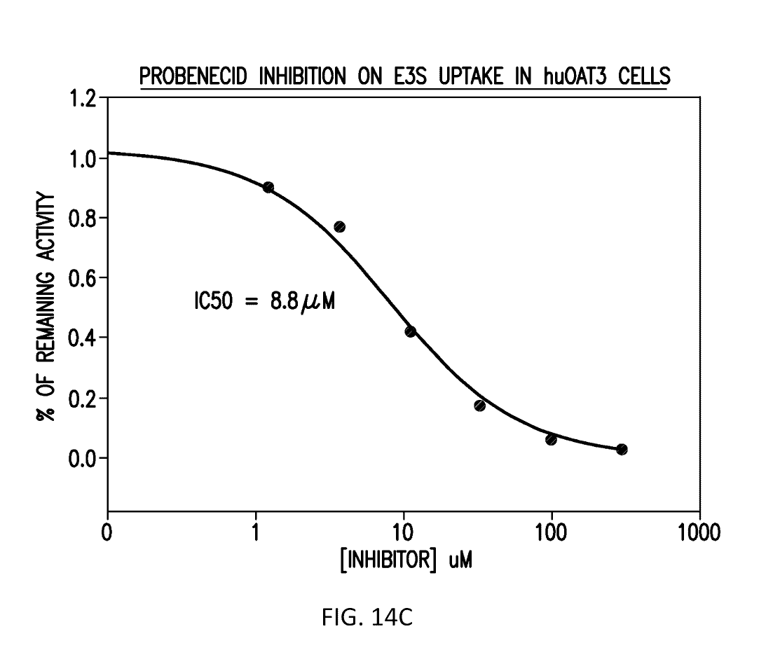

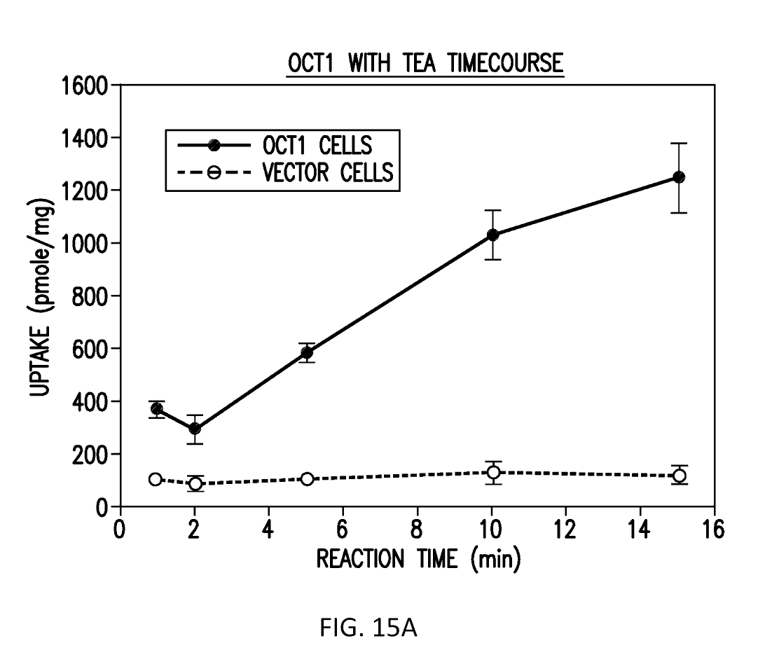

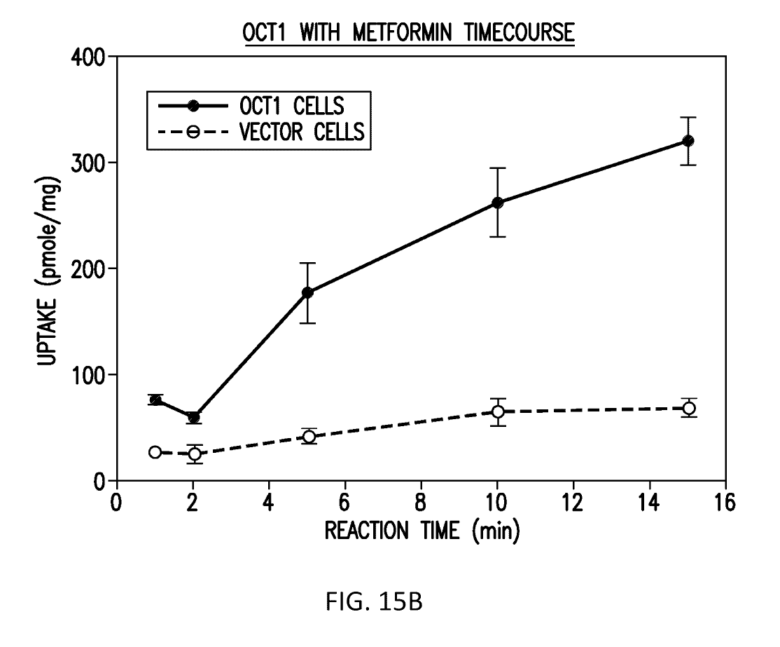

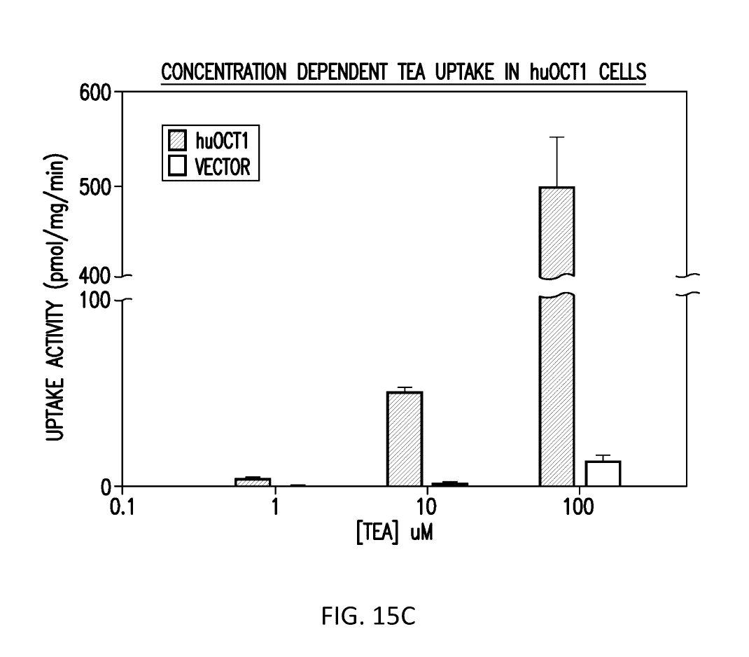

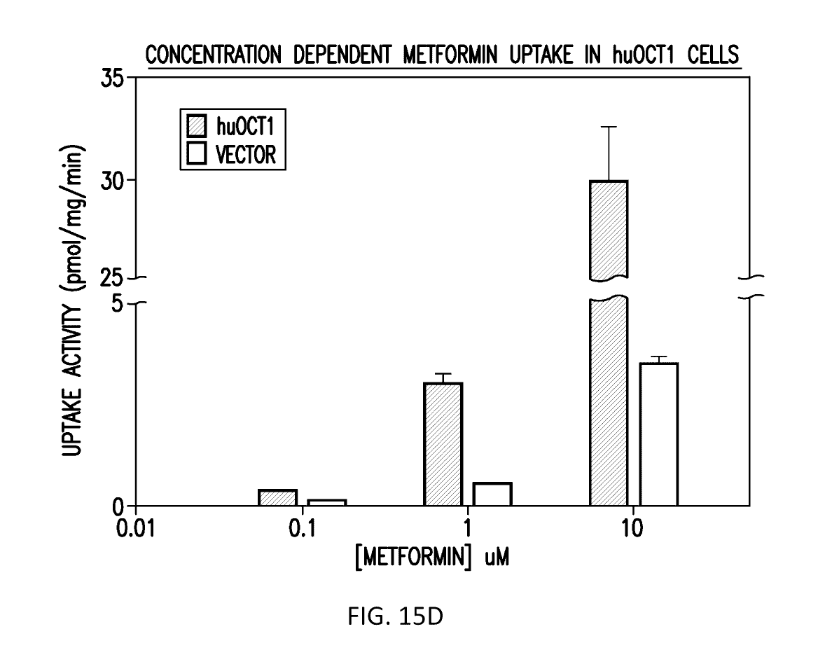

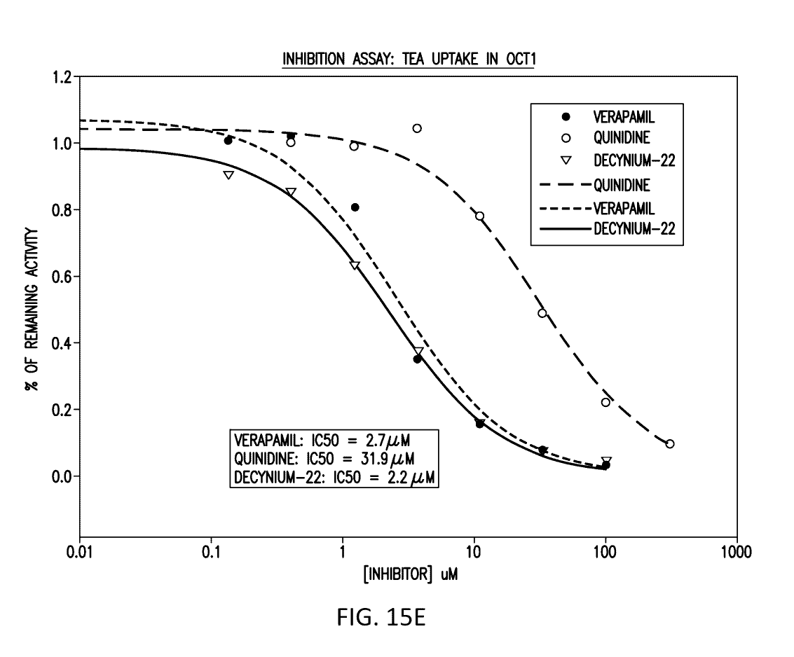

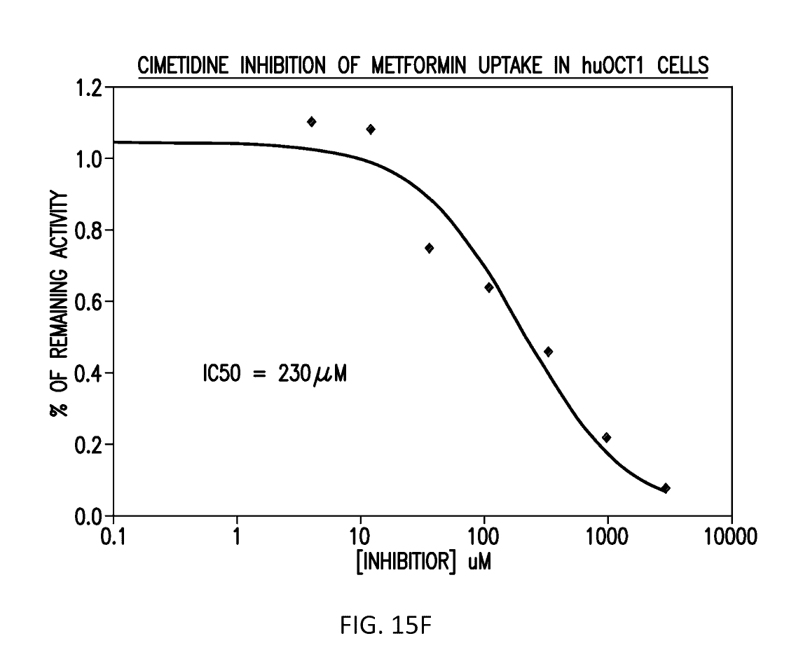

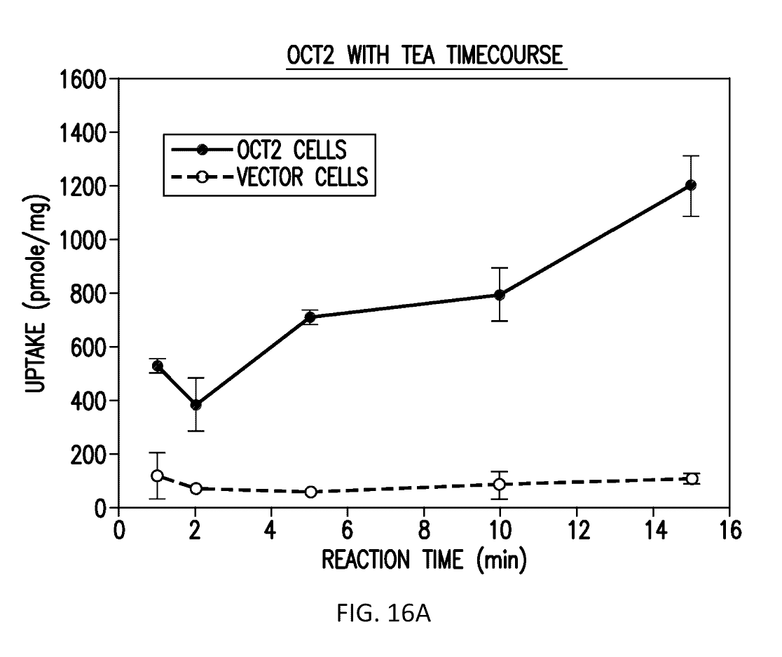

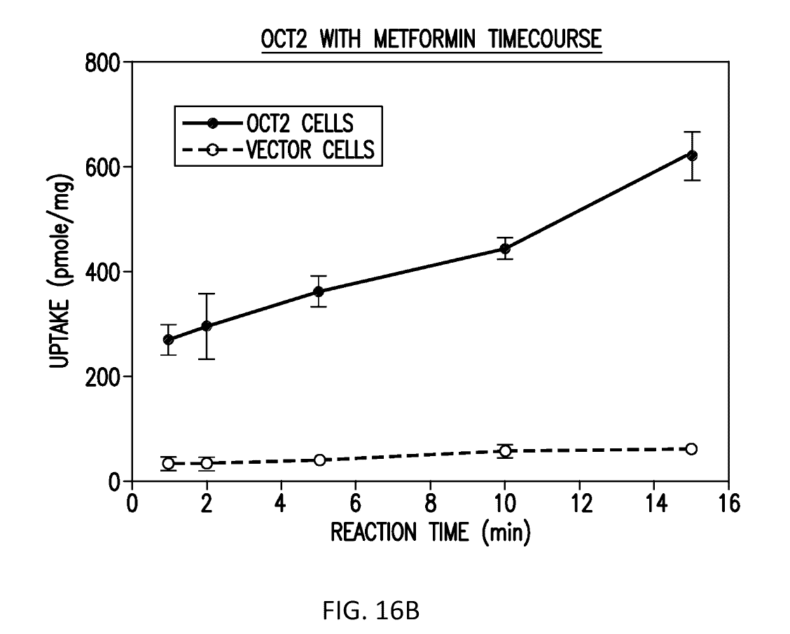

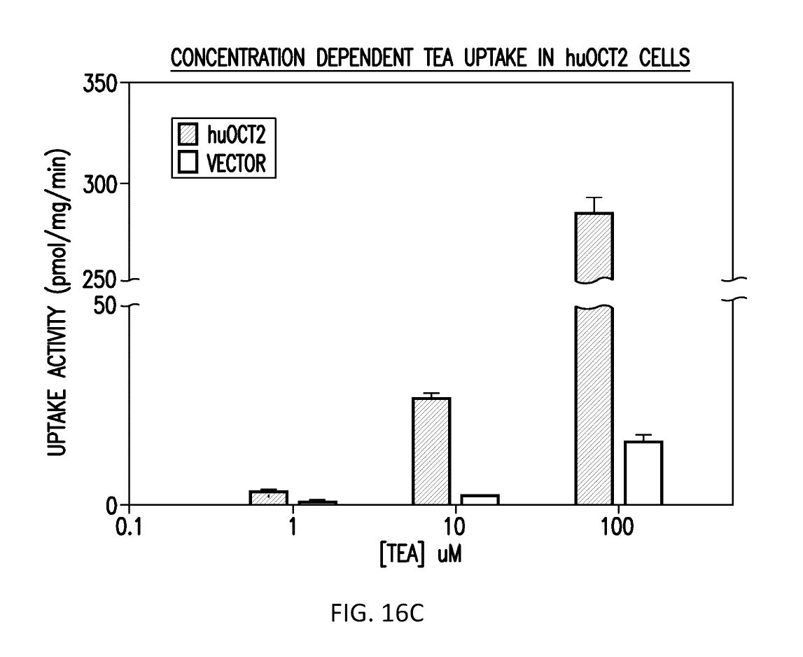

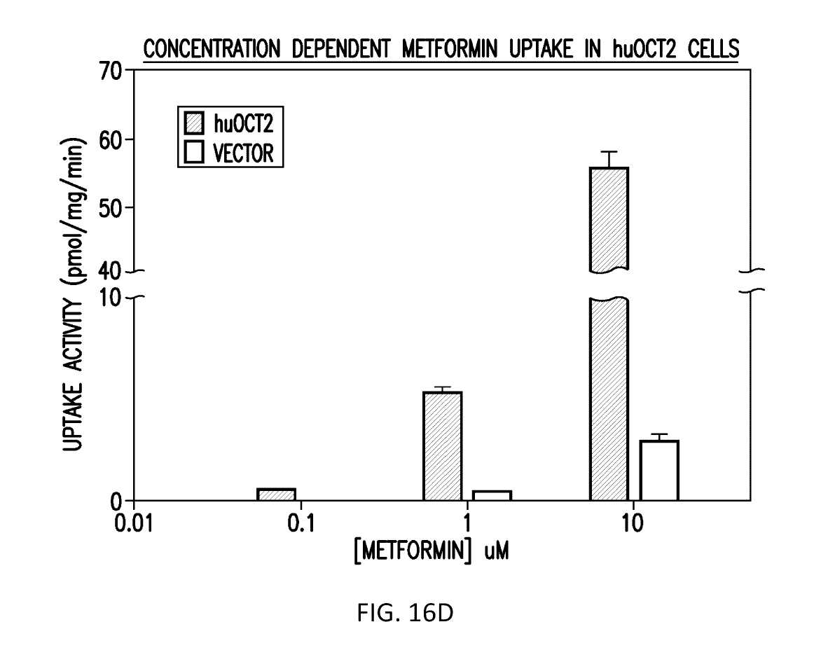

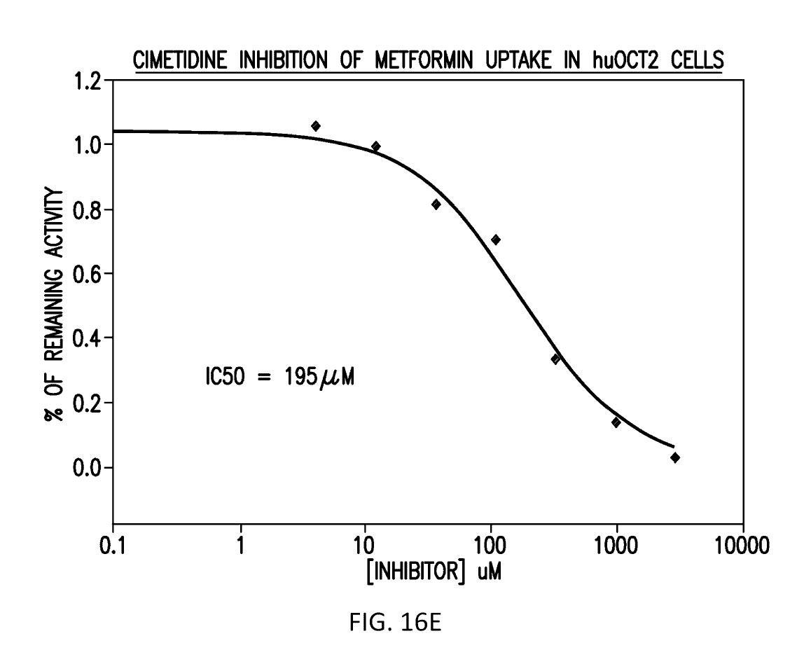

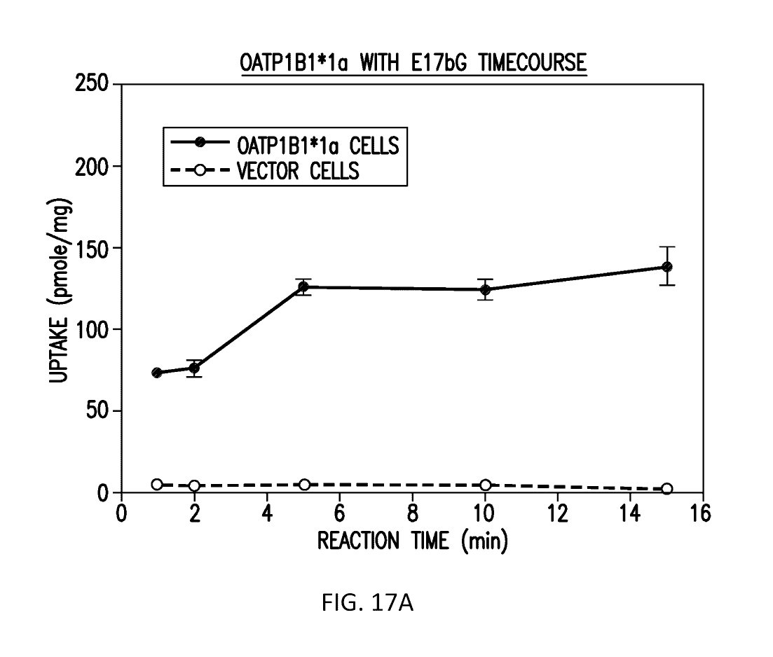

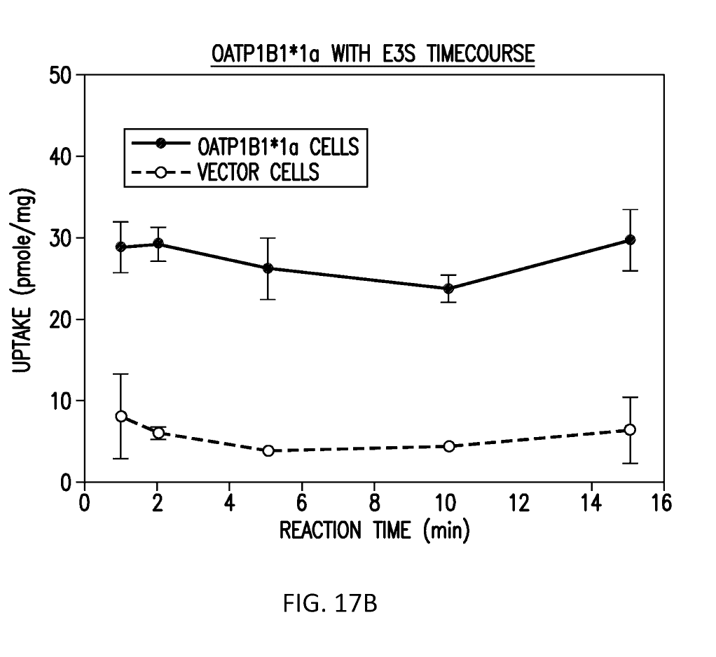

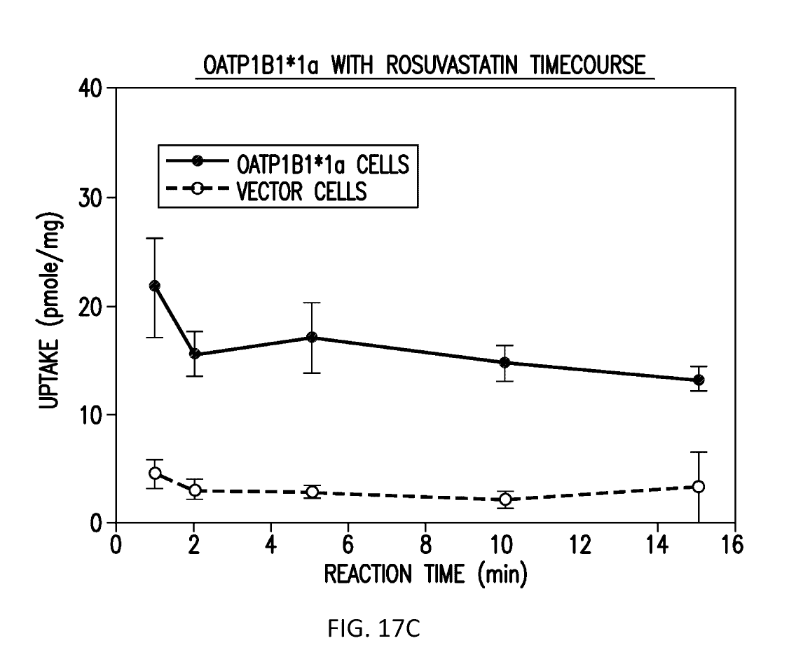

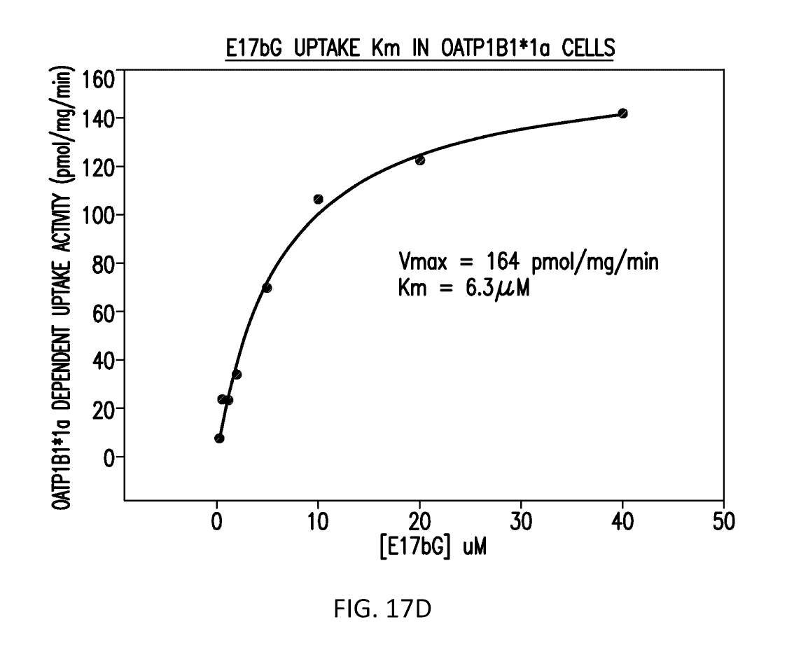

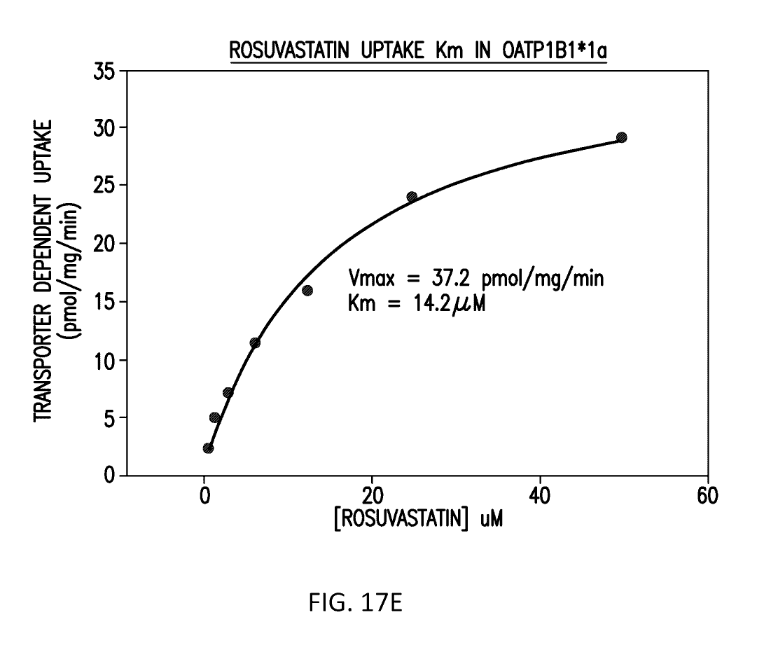

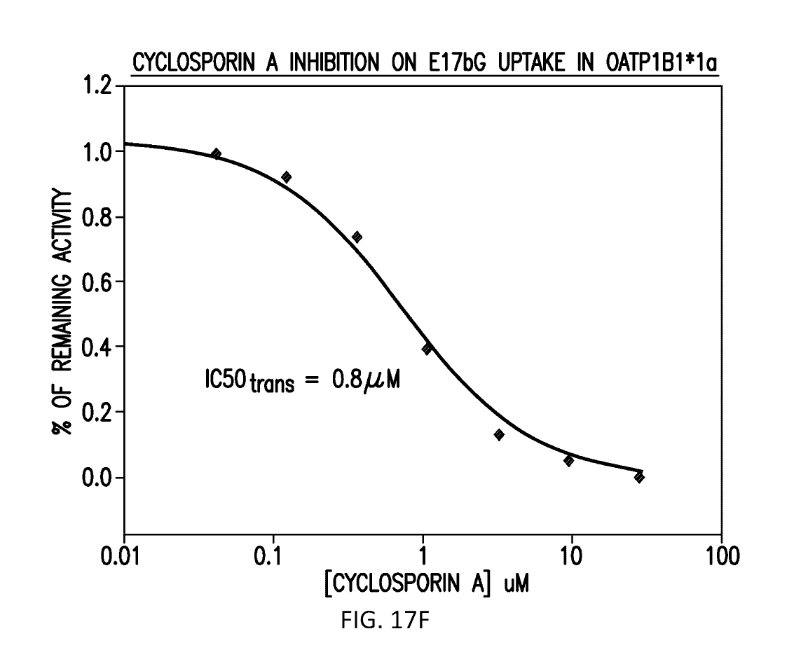

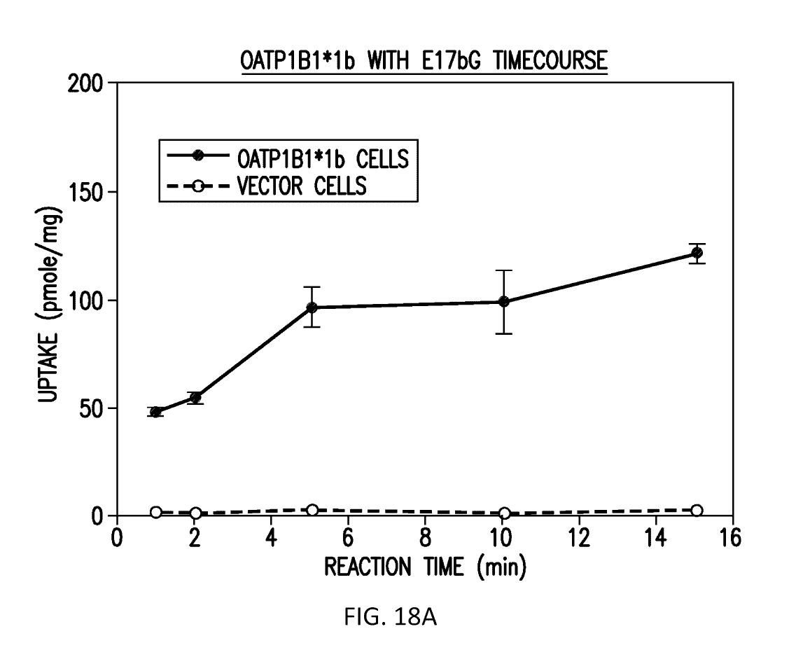

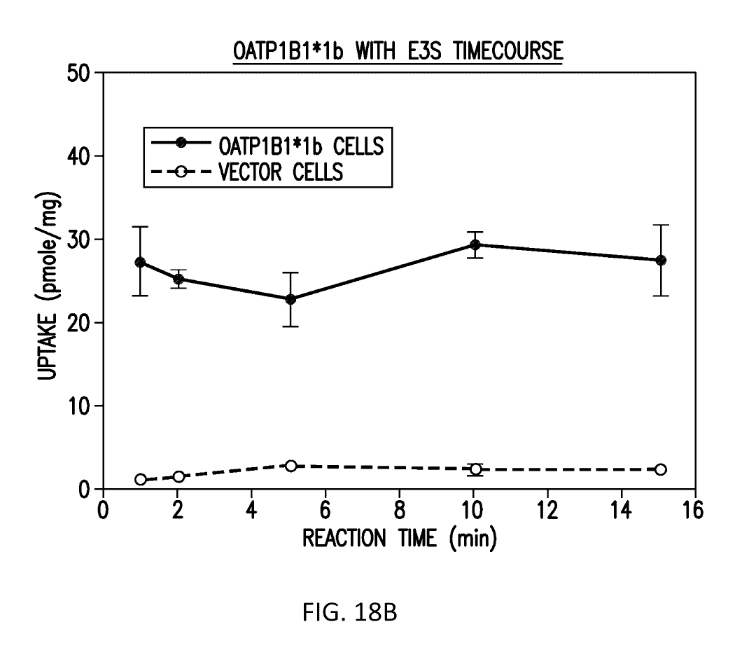

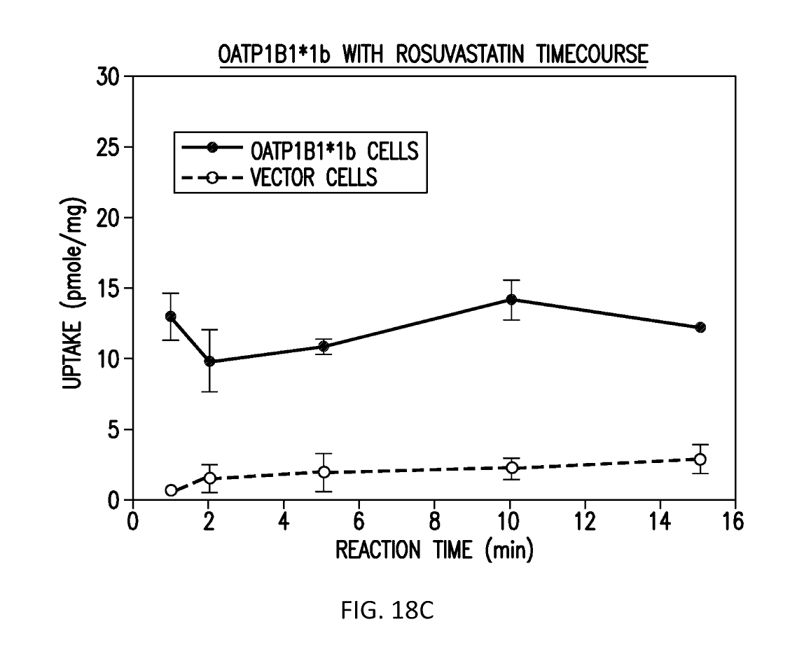

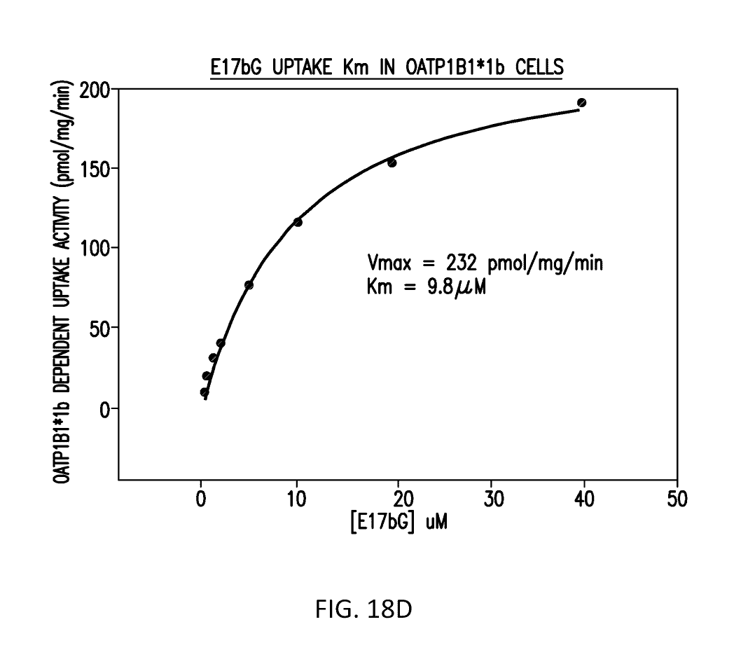

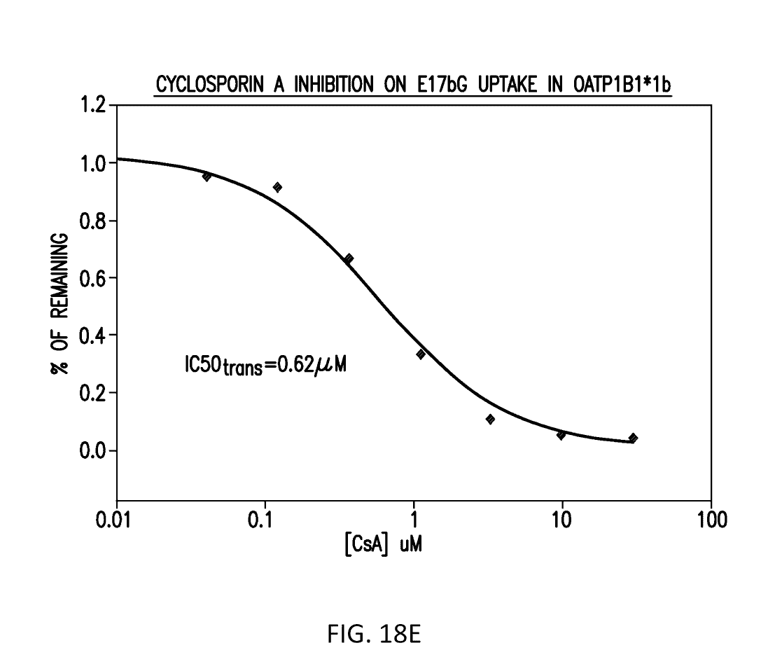

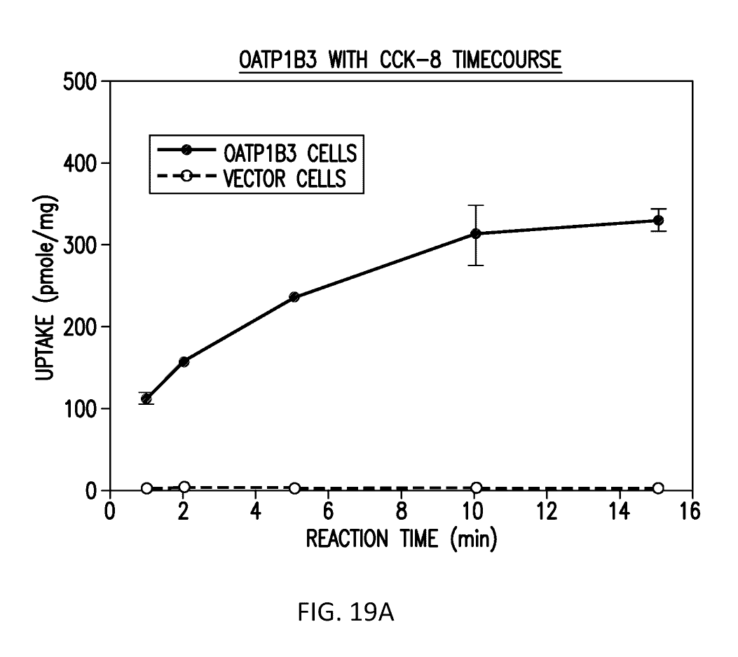

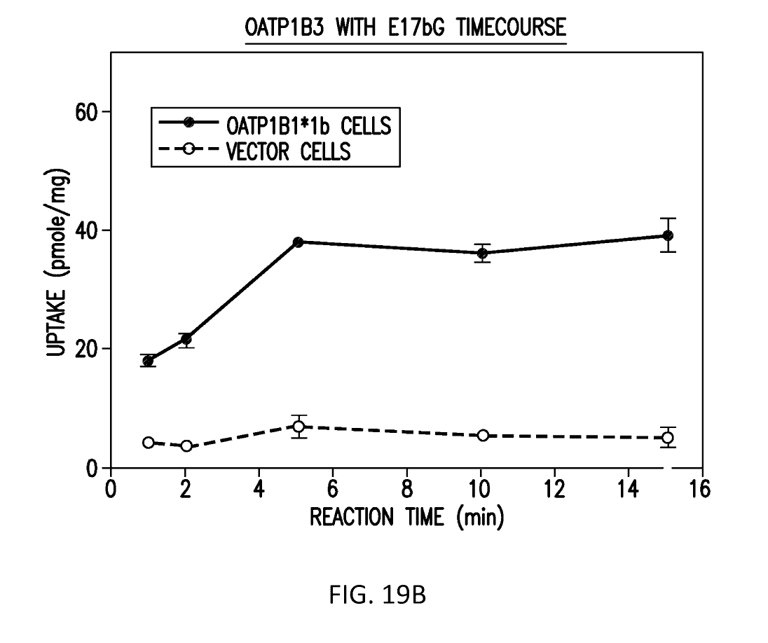

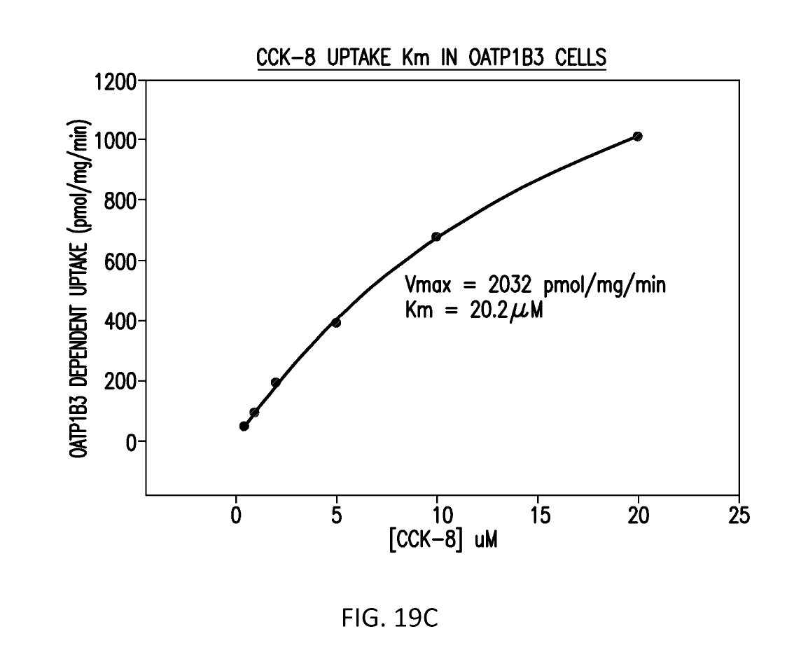

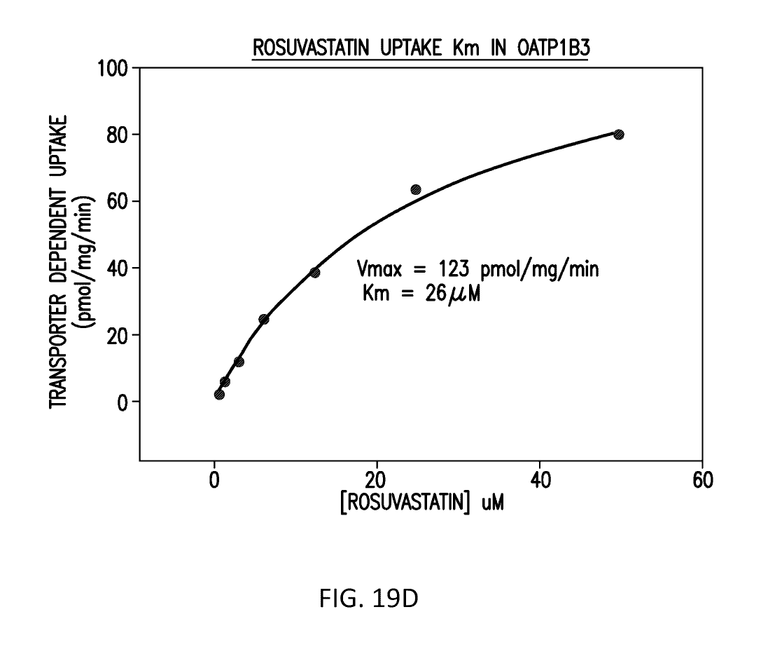

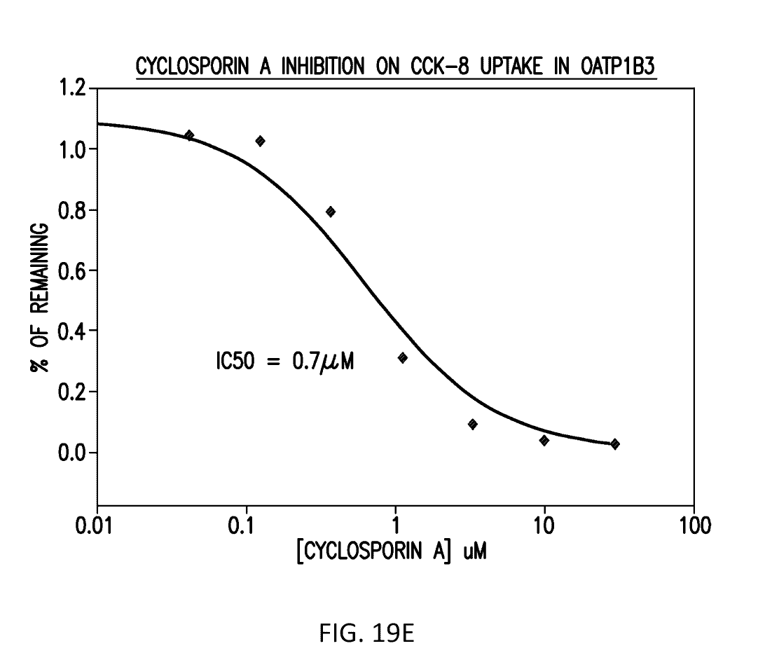

As illustrated in FIGS. 13A-19E and Tables 13-14, the kinetic and inhibition profiles examined in cryopreserved recombinant cells expressing a transporter protein was consistent with reported values. Specifically, as illustrated in FIGS. 13A-13C, the kinetics of PAH uptake by recombinant cells expressing OAT1 and inhibition profile of probenecid thereof is consistent with reported values. As illustrated in FIGS. 14A-14C, the kinetics of E3S uptake by recombinant cells expressing OAT3 and inhibition profile of probenecid thereof is consistent with reported values. As illustrated in FIGS. 15A-15F, the kinetics of TEA and metformin uptake by recombinant cells expressing OCT1 as well as inhibition profile thereof is consistent with reported values. As illustrated in FIGS. 16A-16E, the kinetics of TEA and metformin uptake by recombinant cells expressing OCT2 as well as inhibition profile is consistent with reported values. As illustrated in FIGS. 17A-17F, the kinetics of E17βG, E3S and rosuvastatin uptake by recombinant cells expressing OATP1B1*1a as well as inhibition profile of E17βG uptake by cyclosporin A is consistent with reported values. As illustrated in FIGS. 18A-18E, the kinetics of E17βG, E3S and rosuvastatin uptake by recombinant cells expressing OATP1B1*1b as well as inhibition profile of E17βG uptake by cyclosporin A is consistent with reported values. As illustrated in FIGS. 19A-19E and Tables 13-14, the kinetics of CCK-8, E17βG and rosuvastatin uptake by recombinant cells expressing OATP1B3 as well as inhibition profile of CCK-8 uptake by cyclosporin A is consistent with reported values.

TABLE 13

| SLC Transporter Cells | | | Literature Report | | |
|---|---|---|---|---|---|
| | | Kₘ | Kₘ | Test | |
| Transporter | Substrate | (μM) | (μM) | System | Literature |
| OATP1B1*1a | E17βG | 6.2 | 6.3 | HEK293 | P. Sharma, et al. |
| | | | | cells | Xenobiotica |
| | | | | | 40:24. 2010 |
| OATP1B3 | CCK-8 | 20.2 | 16.5 | CHO | Poirier A, et al., J |
| | | | | Cells | Pharmacokinet |
| | | | | | Pharmacodyn, |
| | | | | | 2009 |
| OAT1 | PAH | 87.3 | 28 | HEK293 | Ueo H, et al., |
| | | | | cells | Biochem |
| | | | | | Pharmacol., 2005 |
| OAT3 | E3S | 4.0 | 6.3 | HEK293 | Ueo H, et al., |
| | | | | cells | Biochem |
| | | | | | Pharmacol., 2005 |

TABLE 14

| Corning ® SLC TransportoCells ™ | | | | Literature Report | | |
|---|---|---|---|---|---|---|
| | | | IC50 | IC50 | Test | |
| Transporter | Substrate | Inhibitor | (μM) | (μM) | System | Literature |
| OATP1B1*1a | E17βG | Cyclosporin A | 0.8 | 0.7 | HEK293 | MG Soars, et |
| | | | | | Cells | al., Drug Metab |
| | | | | | | Dispos, 2012 |
| OATP1B3 | CCK-8 | Cyclosporin A | 0.7 | 0.6 | HEK293 | Bednarczyk D. |
| | | | | | Cells | Anal Biochem. |
| | | | | | | 2010 |
| OAT1 | PAH | Probenecid | 7.2 | 6.5 | CHO | Ho ES, et al., J |
| | | | | | | Am Soc |
| | | | | | | Nephrol., 2001 |
| OAT3 | E35 | Probenecid | 8.8 | 9 | S2 | Takeda M, et |
| | | | | | | al., Eur J |
| | | | | | | Pharmacol., |
| | | | | | | 2001 |
| OCT1 | Metformin | Cimetidine | 230 | 104 | HEK293 | Sumito I, et al., |
| | | | | | Cells | JPET, 2011 |
| OCT2 | Metformin | Cimetidine | 195 | 124 | HEK293 | Sumito I, et al., |
| | | | | | Cells | JPET, 2011 |

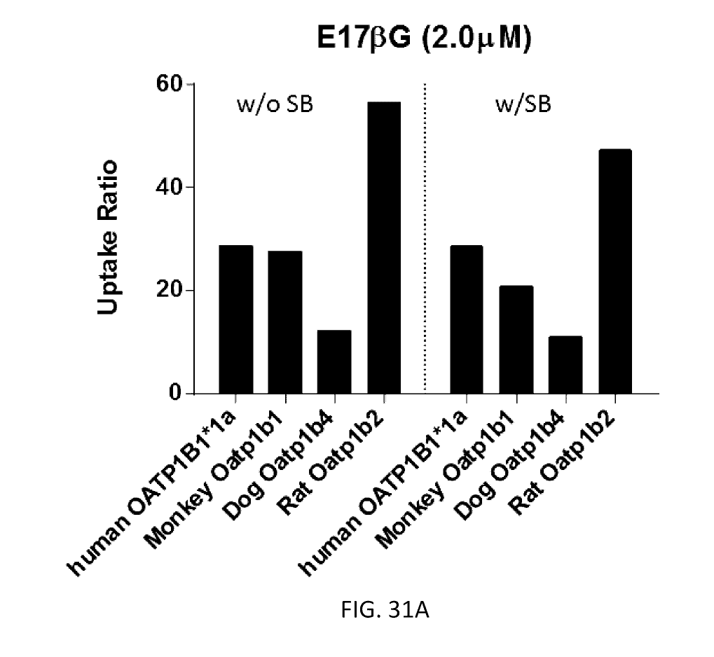

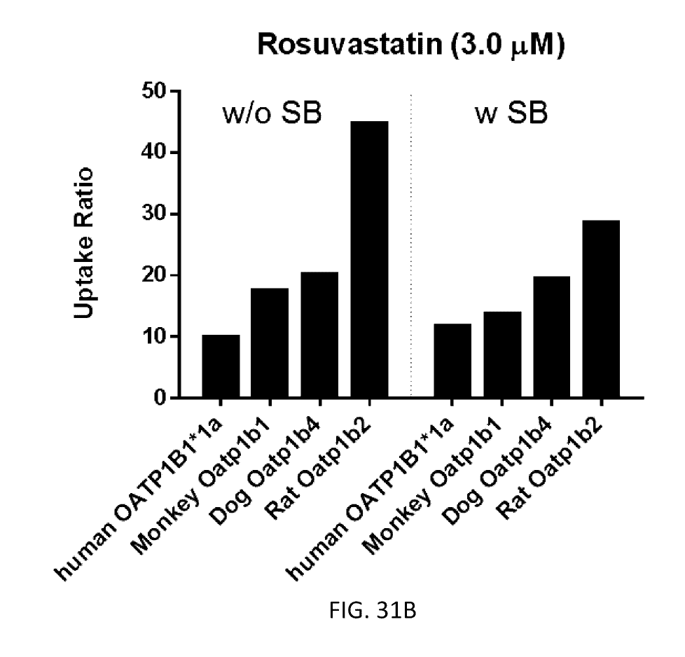

As previously discussed, rats, dogs and monkeys are all frequently used in preclinical testing in order to study early pharmacokinetics (i.e., ADME) and toxicity of potential new drugs. Therefore, the uptake of both E17βG and rosuvastatin was studied in both the presence and absence of sodium butyrate (“SB”) in HEK-293 cells that overexpressed: (i) monkey Oatp1b1; (ii) dog Oatp1b4; and (iii) rat Oatp1b2, as compared to (iv) human OATP1B1*1a (i.e., wild-type). The results were graphed and are shown in FIG. 31A and FIG. 31B. As shown in FIG. 31A and FIG. 31B, monkey Oatp1b1, dog Oatp1b4 and rat Oatp1b2 all show significant uptake of both E17βG and rosuvastatin, considered to be prototypical substrates. Thus, monkey Oatp1b1, dog Oatp1b4 and rat Oatp1b2, together with human OATP1B, enable mechanistic studies to better understand, study and compare drug clearance in different species.

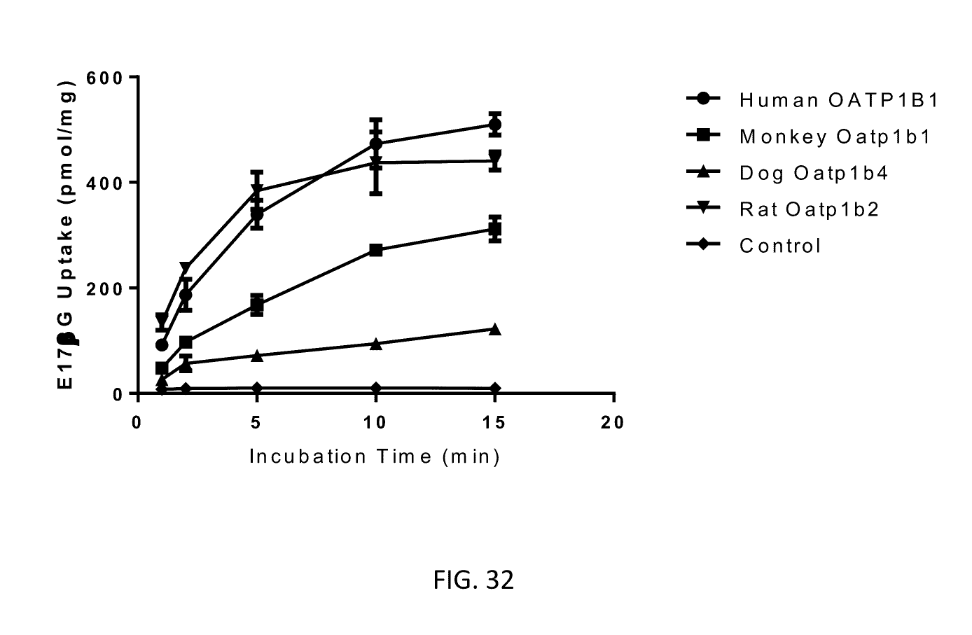

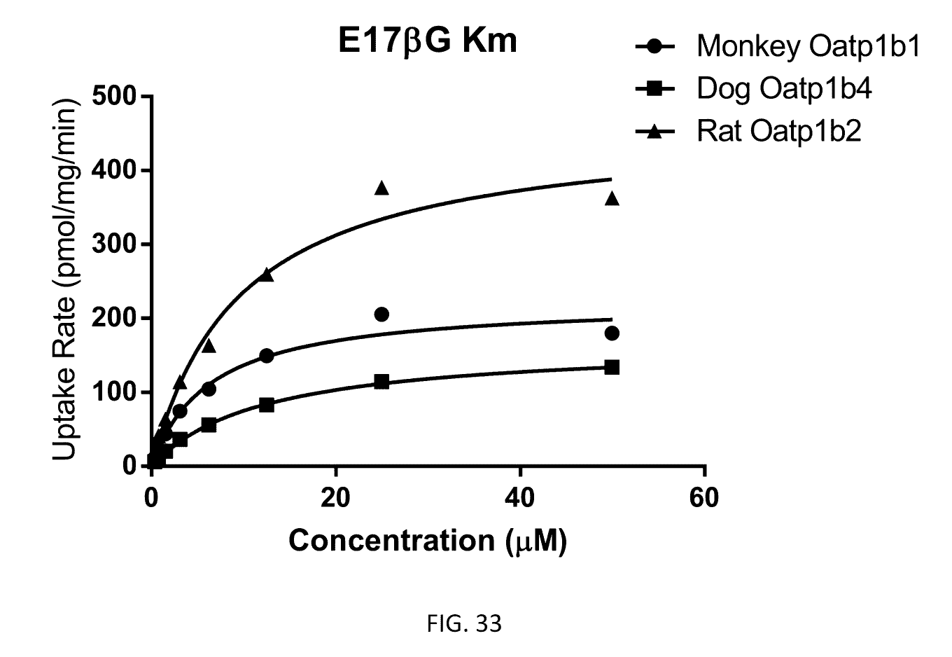

Additional assays of animal species were conducted. First, a time-course experiment was conducted to demonstrate the time-dependent uptake of the probe substrate via OATP/Oatps. Uptake of 2.0 μM estradiol-17β-glucuronide in human OATP1B1*1a, monkey Oatp1b1, dog Oatp1b4, and rat Oatp1b2 cells were determined at 1, 2, 5, 10, and 15 minutes, respectively at 37° C. The results are shown in FIG. 32. Additionally, a kinetics assay was conducted of the uptake of E17βG in HEK-293 cells overexpressing monkey Oatp1b1, dog Oatp1b4 and rat Oatp1b2 (following incubation of 5 minutes). Kₘ and Vₘₐₓ values were calculated according to Michaelis-Menten kinetics. The results are shown in FIG. 33 and Table 15:

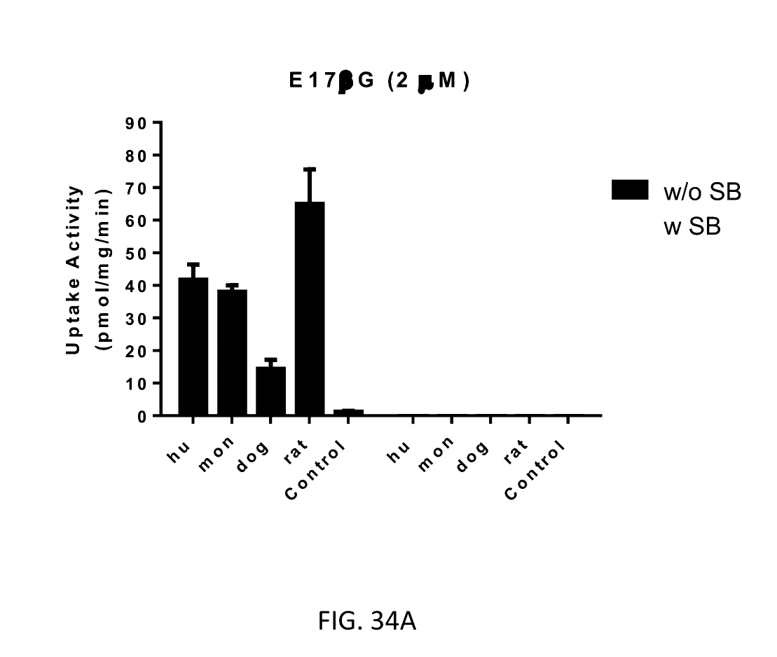

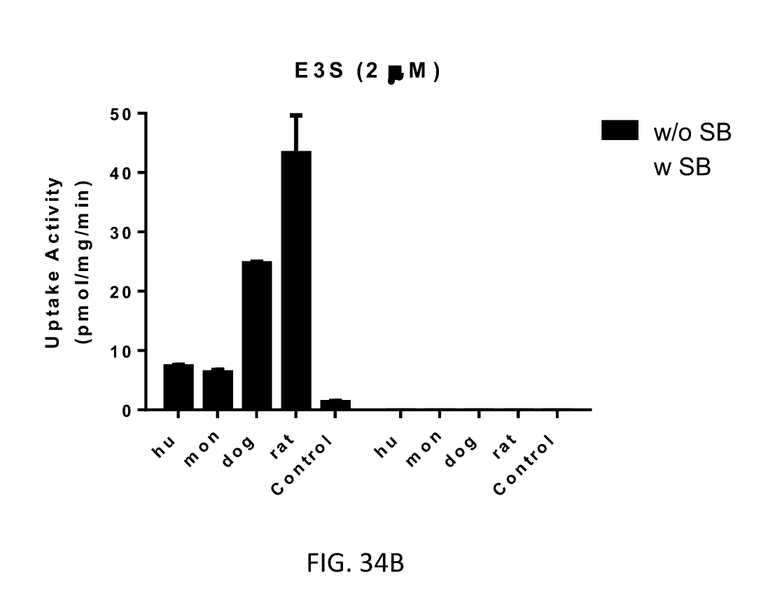

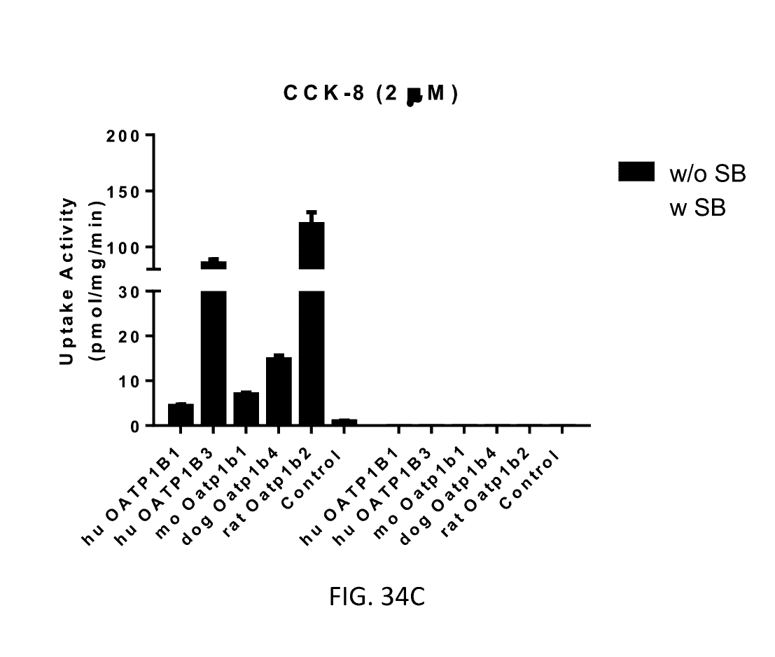

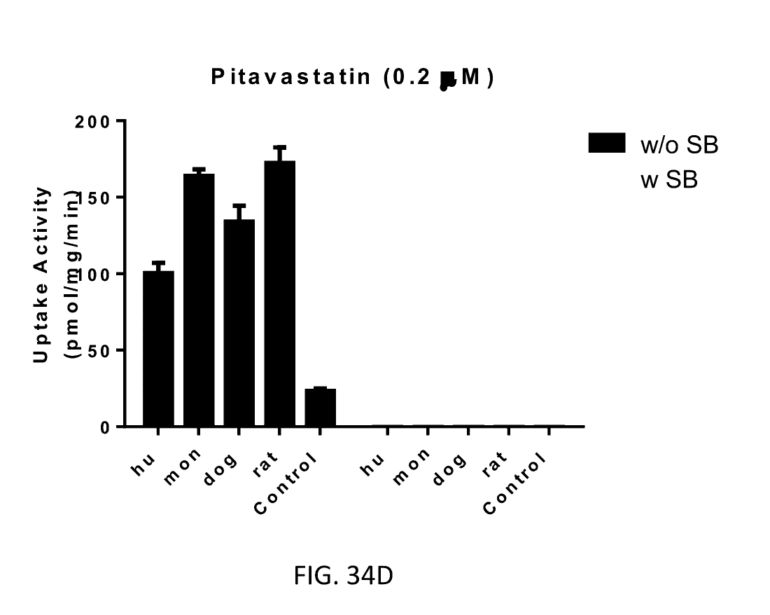

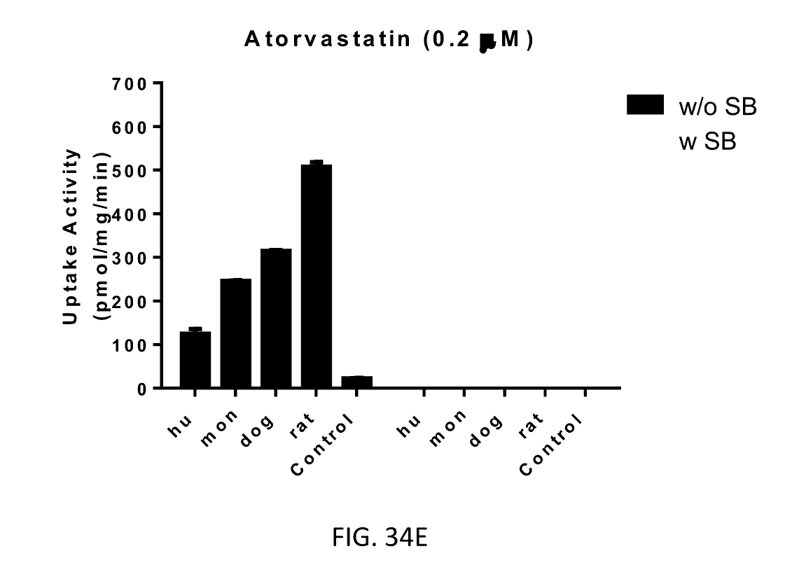

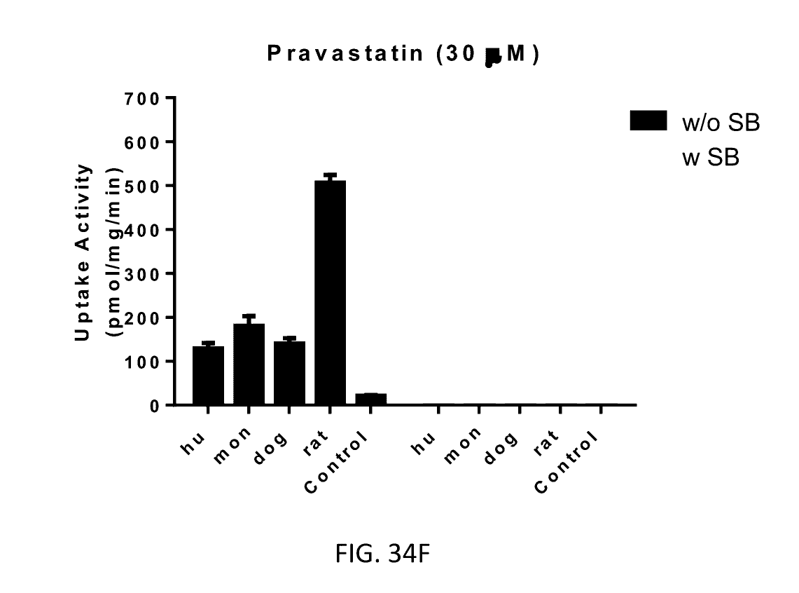

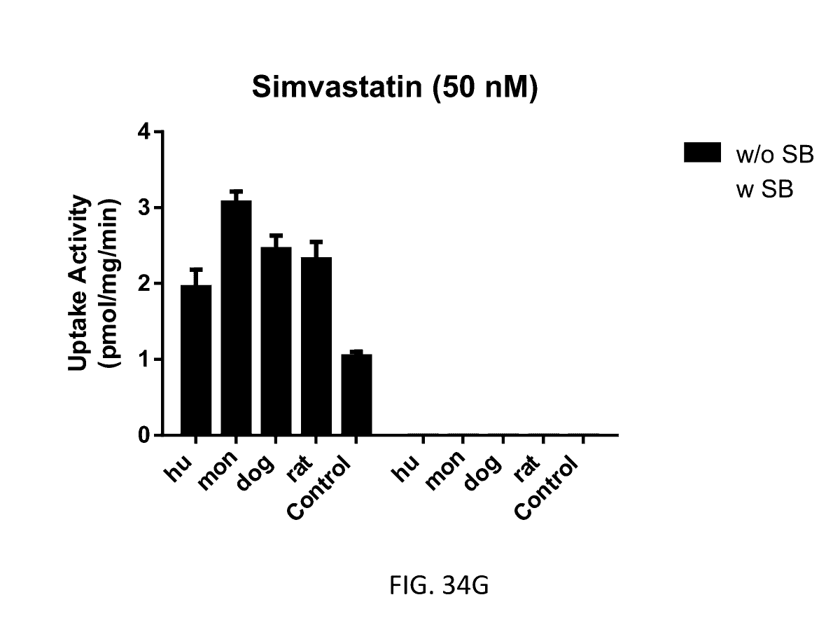

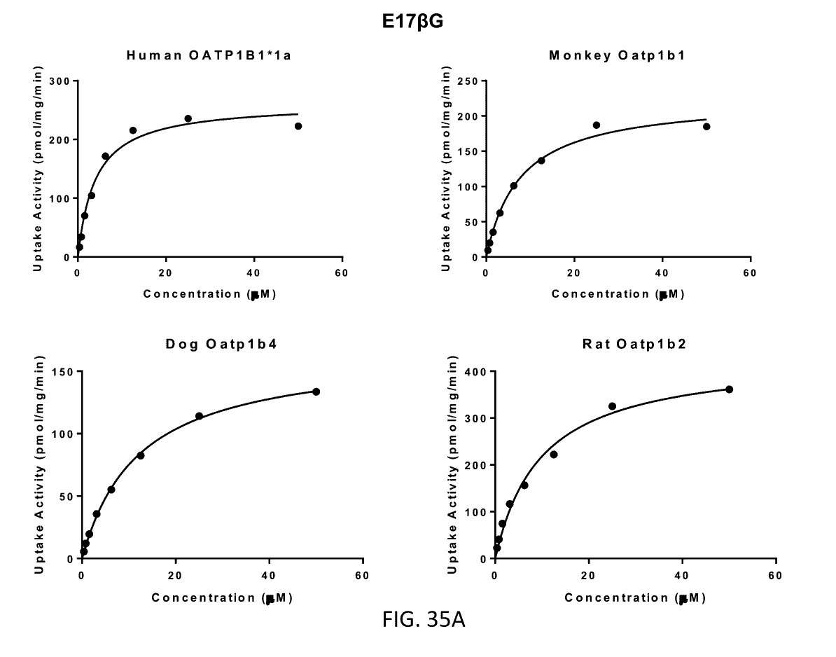

Furthermore, species differences of substrate specificity were examined for prototypical substrates and statins. Human OATP1B1*1a and OATP1B3, monkey Oatp1b1, dog Oatp1b4, rat Oatp1b2, and control cells were incubated with 2 μM estradiol-17β-glucuronide, 2 μM estrone-3-sulfate, or 2 μM CCK-8 for 5 minutes at 37° C.; 0.2 μM pitavastatin, 0.2 μM atorvastatin, 30 μM pravastatin, or 50 nM simvastatin for 2 minutes at 37° C. The results are shown in FIG. 34A-FIG. 34G, respectively. Data is shown as the mean±S.D. of three replicates (n=3). Significant species differences were observed between human OATP1B1 and preclinical species Oatp1bs. Monkey Oatp1b1 demonstrated similar substrate specificity as human OATP1B1; dog Oatp1b4 and rat Oatp1b2 functions like human OATP1B3, as they both showed significant uptake of CCK-8. Compared to other species, dog Oatp1b4 demonstrated similar or higher uptake of all tested substrates, except E17βG; rat Oatp1b2 demonstrated similar (pitavastatin and simvastatin) or significantly higher activity for three prototypical substrates, atorvastatin and pravastatin.

TABLE 15
E17βG

| | | Vₘₐₓ |
|---|---|---|
| Transporter | Kₘ (μM) | (pmol/mg/min) |
| Monkey | 6.3 ± 1.4 | 223 ± 16 |
| Oatp1b1*1a | | |
| Dog Oatp1b4 | 12.2 ± 0.6 | 167 ± 3.1 |
| Rat Oatp1b2 | 9.6 ± 1.9 | 463 ± 34 |

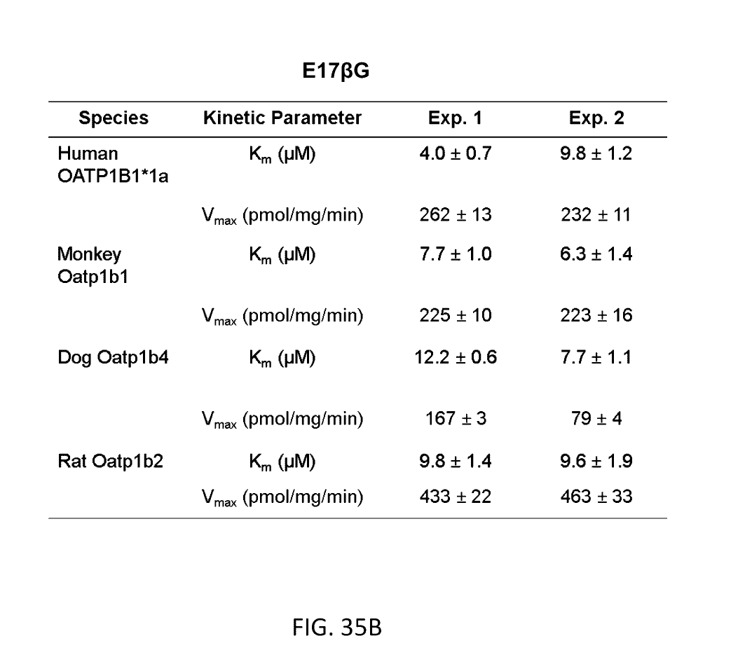

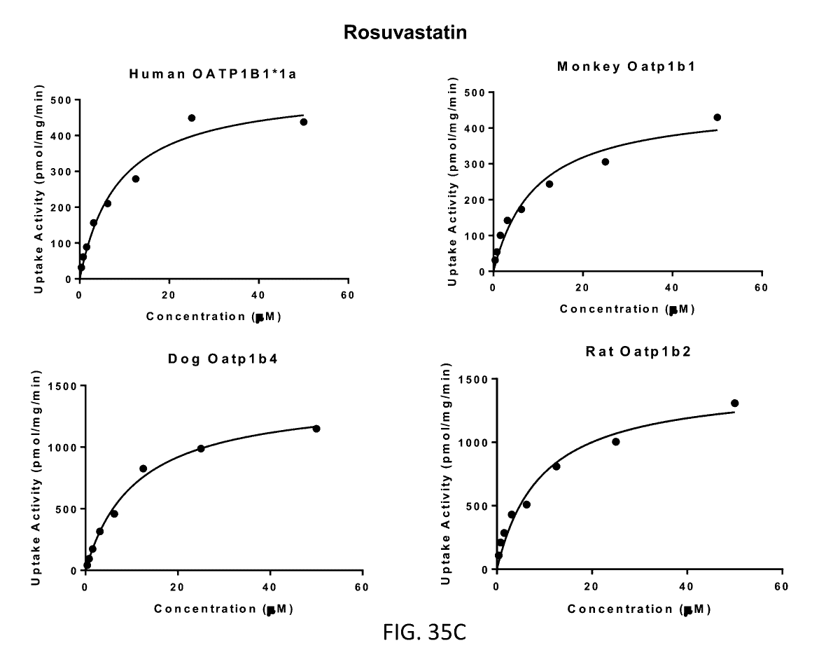

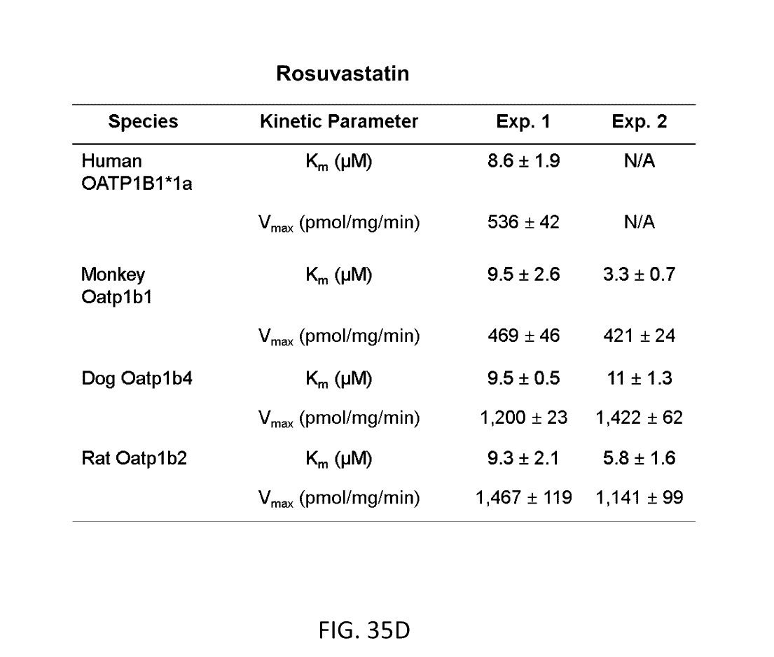

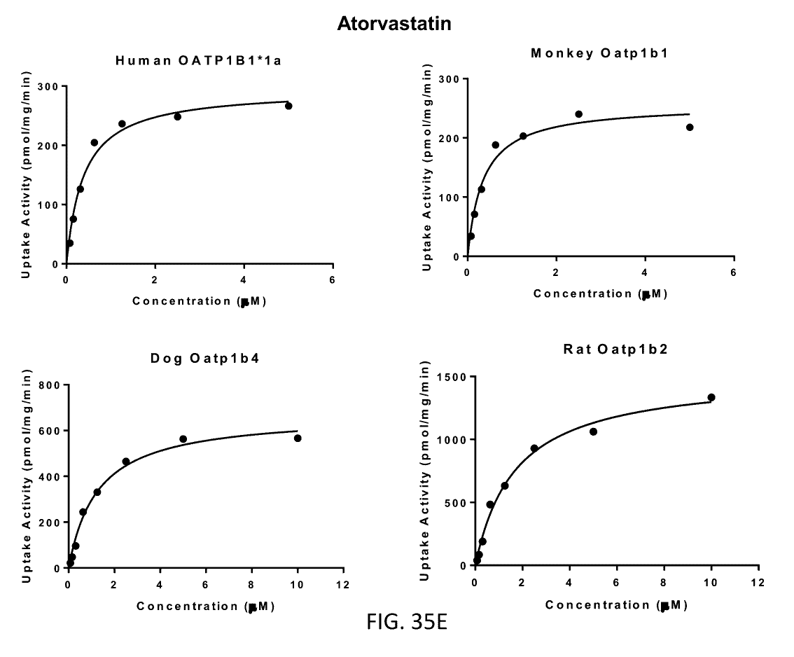

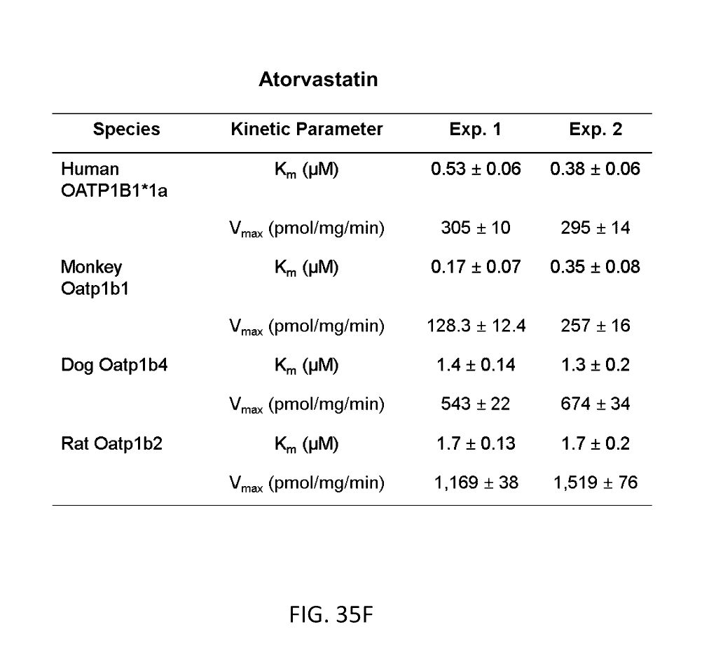

Additionally, kinetic parameters (Kₘ and Vₘₐₓ) were determined for uptake of estradiol-17β-glucuronide (FIG. 35A and FIG. 35B), rosuvastatin (FIG. 35C and FIG. 35D), and atorvastatin (FIG. 35E and FIG. 35F) in human OATP1B1*1a, monkey Oatp1b1, dog Oatp1b4, and rat Oatp1b2 after a 2-minute incubation at 37° C. Control cells were included for all Kₘ and Vₘₐₓ determinations. Eight substrate concentrations were used in each kinetic determination. For each substrate concentration, the initial uptake rate was calculated by subtracting the initial rate determined in HEK cells expressing an empty vector from those obtained in HEK-293 overexpressing SLC transporter. Each point is an average of triplicate determinations. Kinetics were determined in at least two independent experiments and the values are summarized in FIGS. 35B, 35D and 35F.

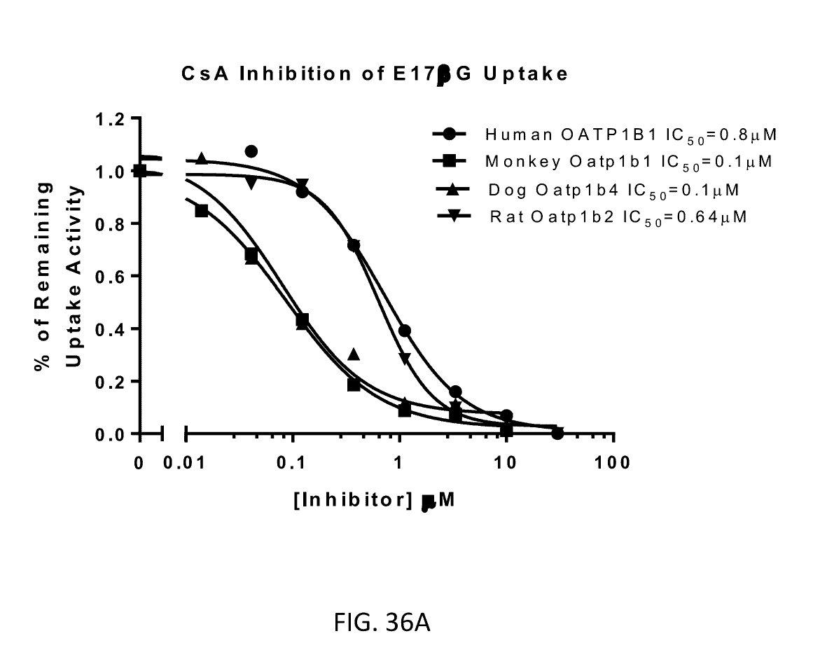

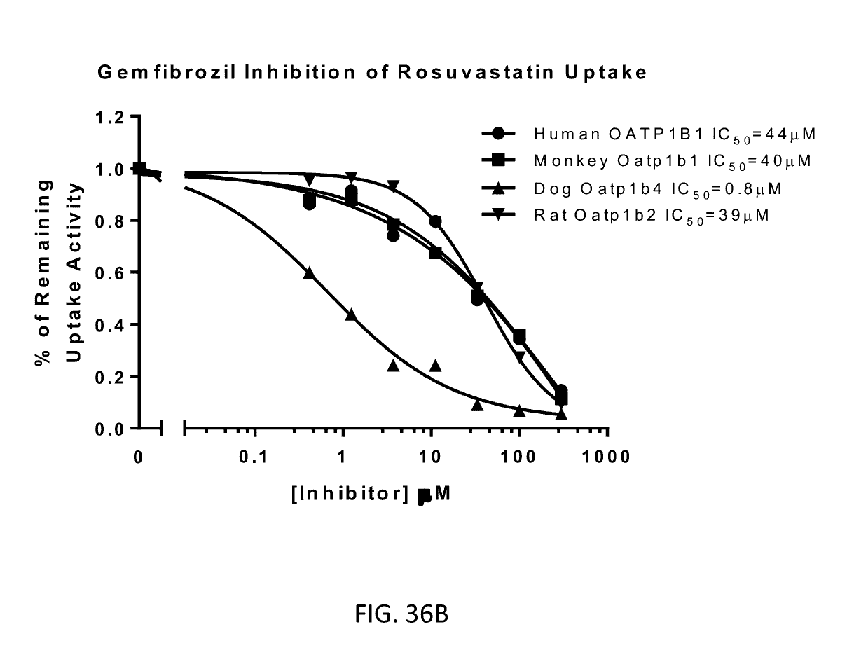

Next, species differences of inhibitory profiles were determined. IC₅₀ values were determined by co-incubating the cells with 1 μM substrates (E17βG or rosuvastatin) with cyclosporin A (FIG. 36A) or gemfibrozil (FIG. 36B) at a range of concentrations. For each inhibitor concentration, the uptake activity was calculated by subtracting uptake activity determined in HEK cells expressing an empty vector from those obtained in HEK overexpressing SLC transporter. Each point represents the mean value of three replicates and the solid lines represented the non-linear regression fitting. The curve represents one of two independent experiments.

### Example 2

### Development of OATP1B1 Single Nucleotide Polymorphism Panel

An OATP1B1 single nucleotide polymorphism panel was developed to allow investigation of drug response by different genetic backgrounds in the early stage of drug development. OATP1B1*1a, OATP1B1*5, and OATP1B1*15 were transiently overexpressed in HEK-293 cells and then cryopreserved. The expression levels of the recombinant proteins were quantitated and normalized in the haplotype cells versus wild type cells by targeted protein quantification via liquid chromatography/tandem mass spectrometry. Uptake of OATP1B1 prototypical substrate, estradiol-17β-glucuronide (E17βG), and statins was determined in OATP1B1*1a, OATP1B1*5, OATP1B1*15, and control cells. E17βG uptake was reduced to 40% to 50% in OATP1B1*5 and *15 cells compared to OATP1B1*1a cells. Significant decrease in uptake activity was observed in OATP1B1*5 and *15 for simvastatin, atorvastatin, pitavastatin, and rosuvastatin, but not for fluvastatin. The results are consistent with the clinical finding of impact of the genotypes on the pharmacokinetics of these statins. The new OATP1B1 single nucleotide polymorphism panel is, therefore, a useful tool to facilitate prediction of drug disposition in populations with different genotypes.

Experimental Protocol and Results.

CORNING® TRANSPORTOCELLS™ OATP1B1*1a (Cat. No. 354859), OATP1B1*5 (Cat. No. 354878), OATP1B1*15 (Cat. No. 354879), control cells (Cat. No. 354854), cell culture media components and assay buffer were obtained from Corning Life Sciences. Radiolabeled and non-radiolabeled chemicals were obtained from American Radiolabeled Chemicals or Sigma-Aldrich.

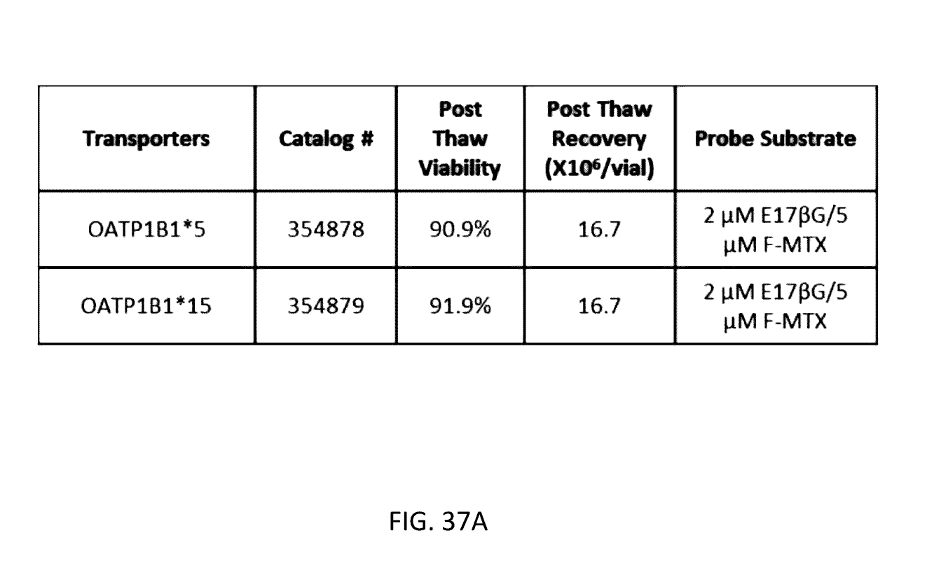

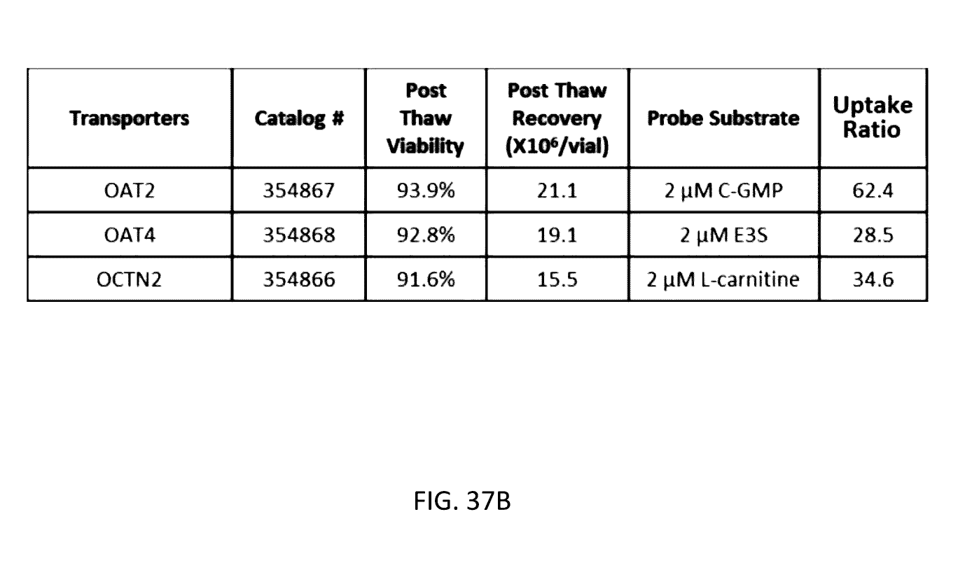

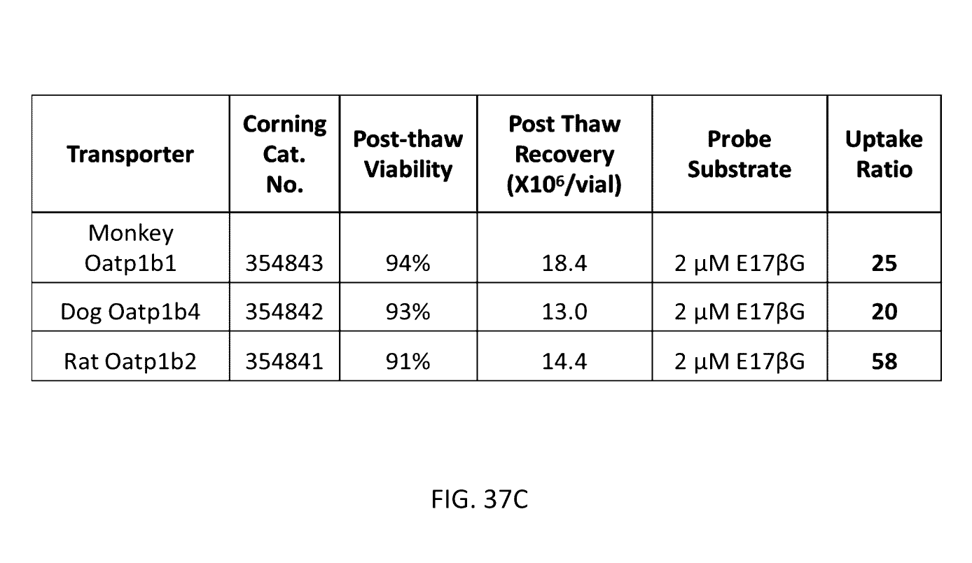

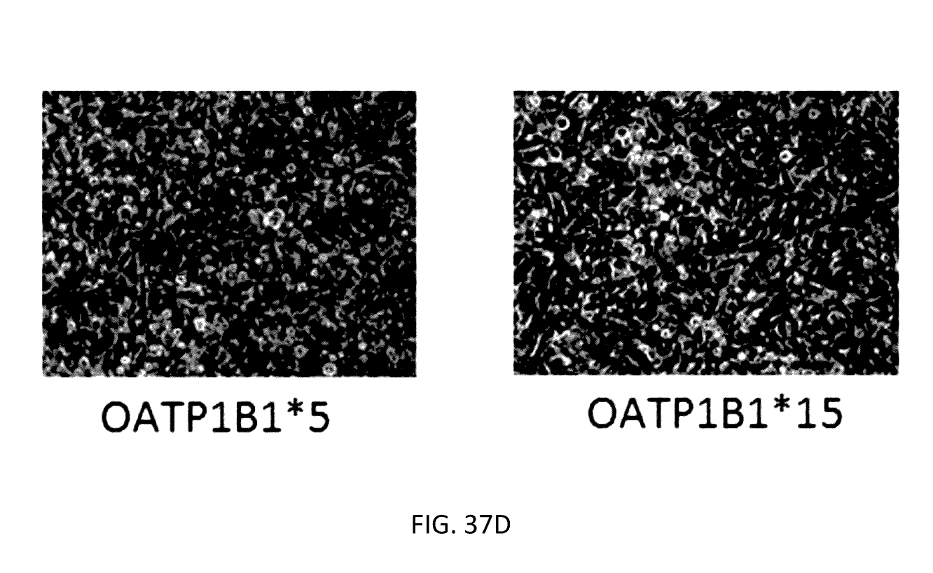

Cells were thawed and plated at a seeding density of 200K per well in 48-well poly-D-lysine coated plates (Corning Life Sciences) according to the manufacturer recommended procedure. The viability and recovery of the thawed OATP1B1*5 (Cat. No. 354878) and OATP1B1*15 cells is illustrated in FIG. 37A. Viability data and Uptake Ratio data for thawed OAT2, OAT4, OCTN2 HEK cells under the same conditions are illustrated in FIG. 37B, while viability data and Uptake Ratio data for thawed monkey Oatp1b1, dog Oatp1b4, and rat Oatp1b2 HEK-293 cells under the same conditions are illustrated in FIG. 37C. FIG. 37D illustrates the cell mophorlogy and plated OATP1B1*5 and OATP1B1*15 cells.

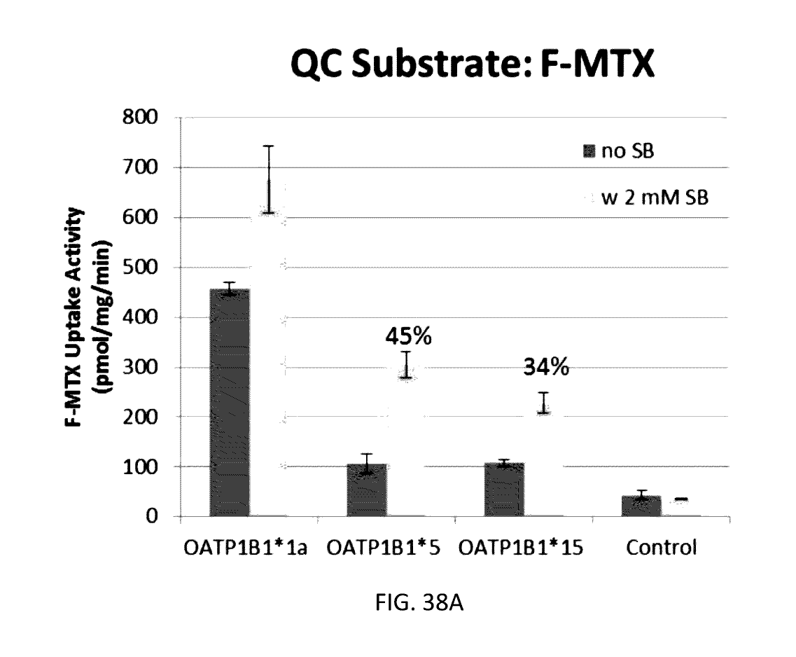

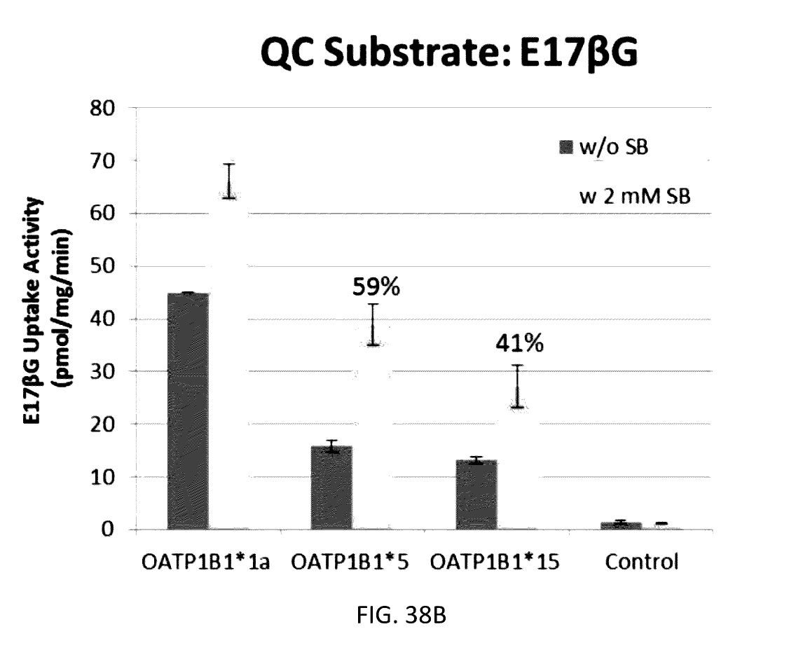

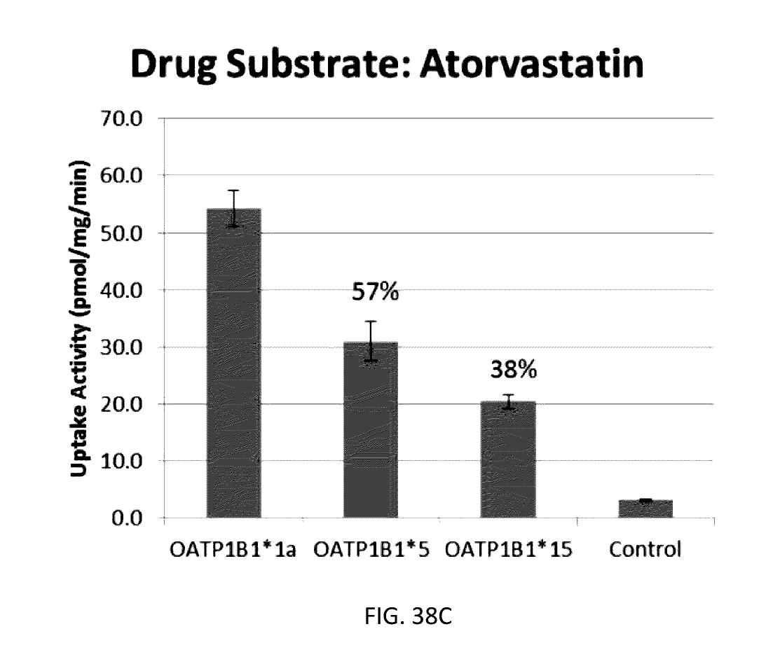

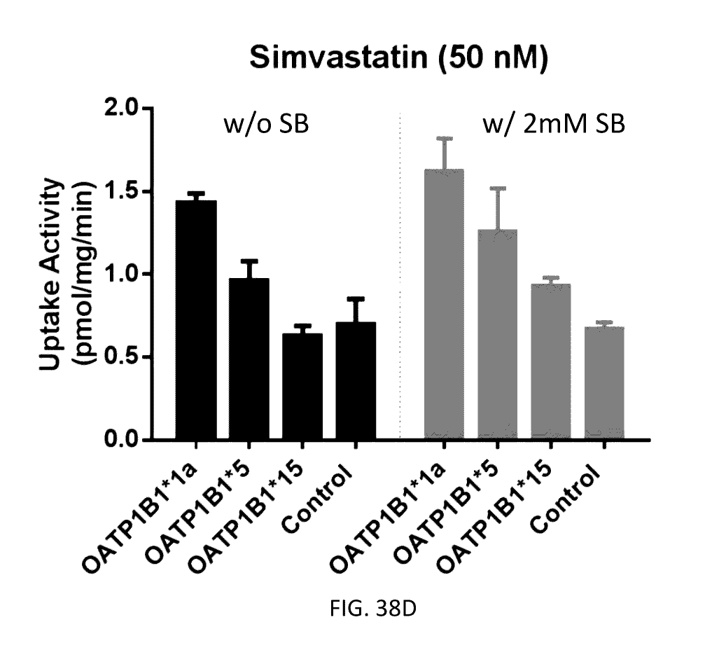

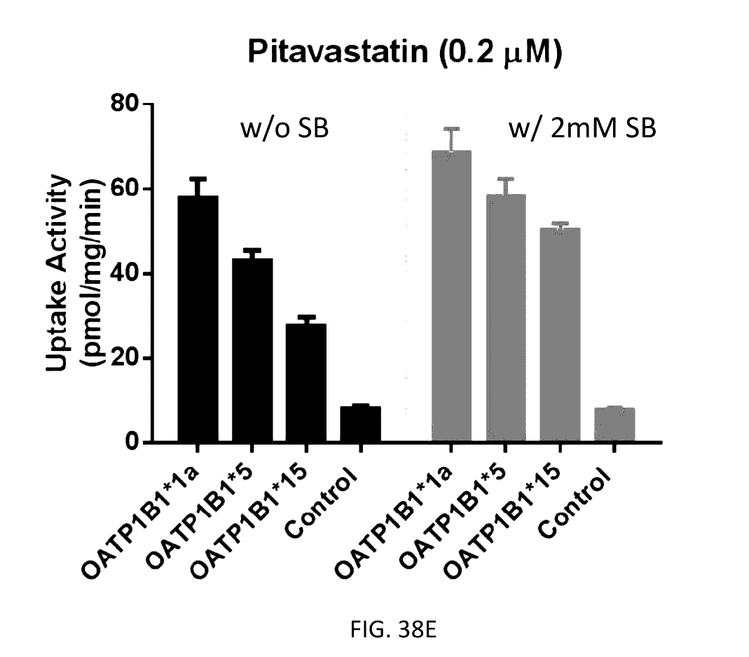

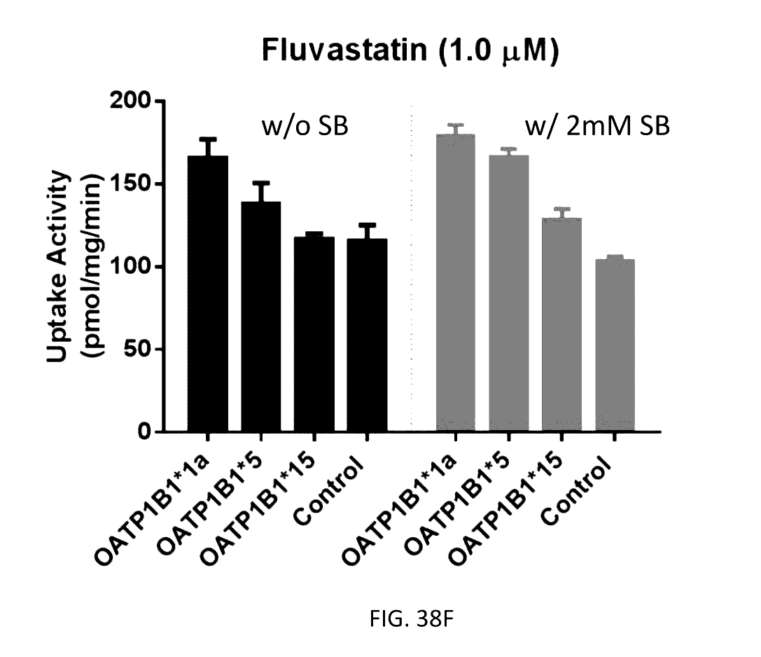

The plated cells were re-fed with or without 2 mM sodium butyrate at 3 to 4 hours after plating. Uptake assays were performed at 24 hours post-plating at 37° C. for F-MTX (5 μM for 10 min), E17βG (2 μM for 5 min), atorvastatin (0.5 μM for 2 min; no SB only), simvastatin (50 nM for 10 min), pitavastatin (0.2 μM for 2 min) and fluvastatin (1.0 μM for 2 min). For radiolabeled compounds, the cells were lysed in M-PER for 5 min at RT, then the cell lysates were ready for analysis. For unlabeled compounds, the cells were lysed in 80% acetonitrile for 20 min at RT, then the cell lysates were analyzed by LC-MS/MS following the method developed previously. The results of the assays are illustrated in FIGS. 38A-38F. Each bar in FIGS. 34A-34F represents the mean and S.D. of triplicate determinations. Under the condition of no SB, uptake of simvastatin was reduced to 36% for *5 and 0% for *15; uptate of pitavastatin was reduced to 70% for *5 and 40% for *15; uptake of fluvastatin was reduced to 46% for *5 and less than 5% for *15.

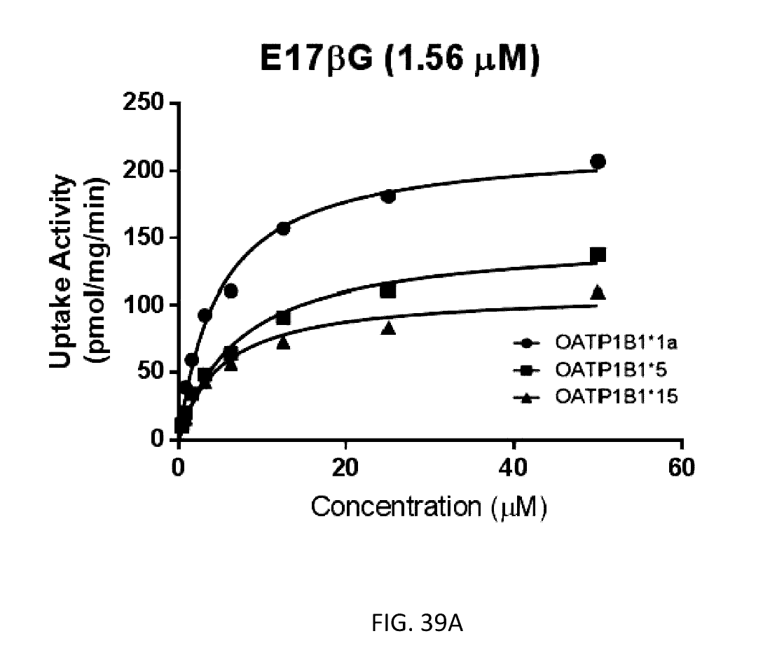

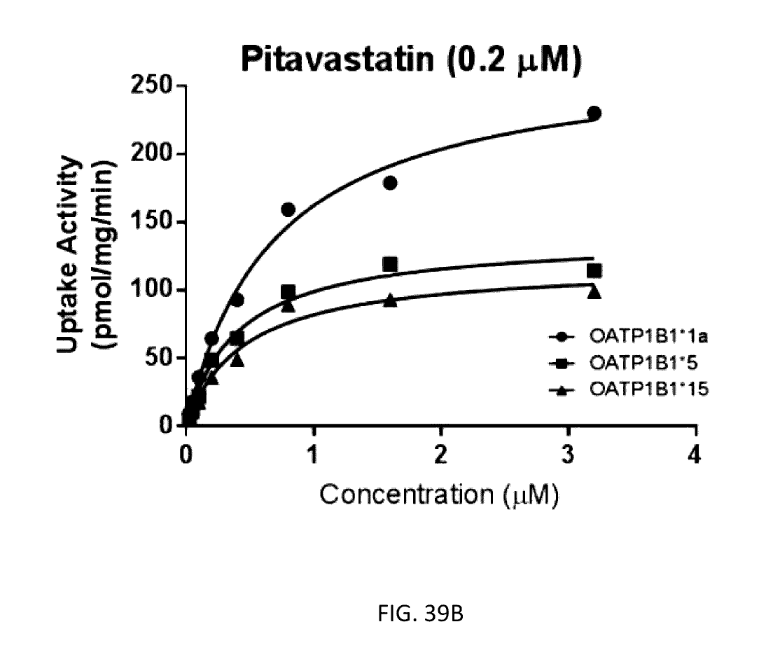

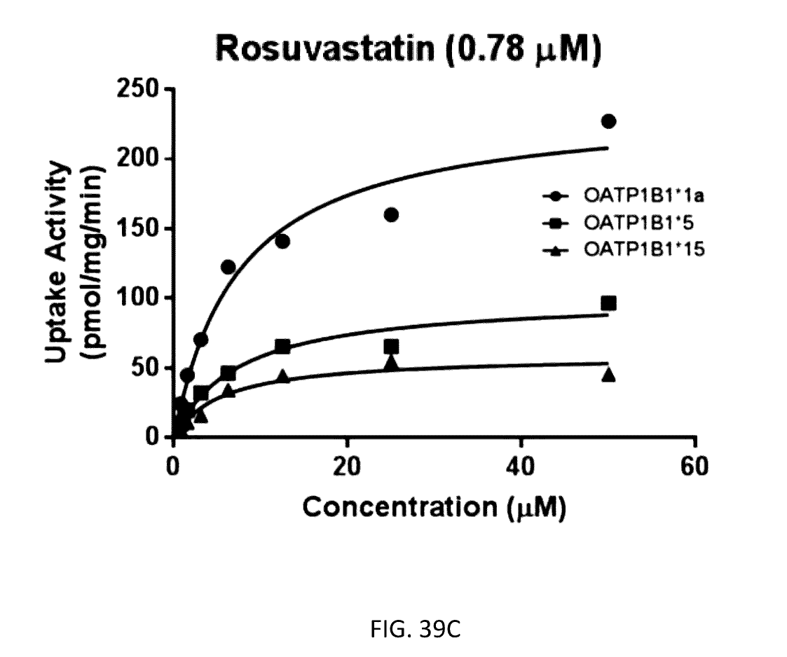

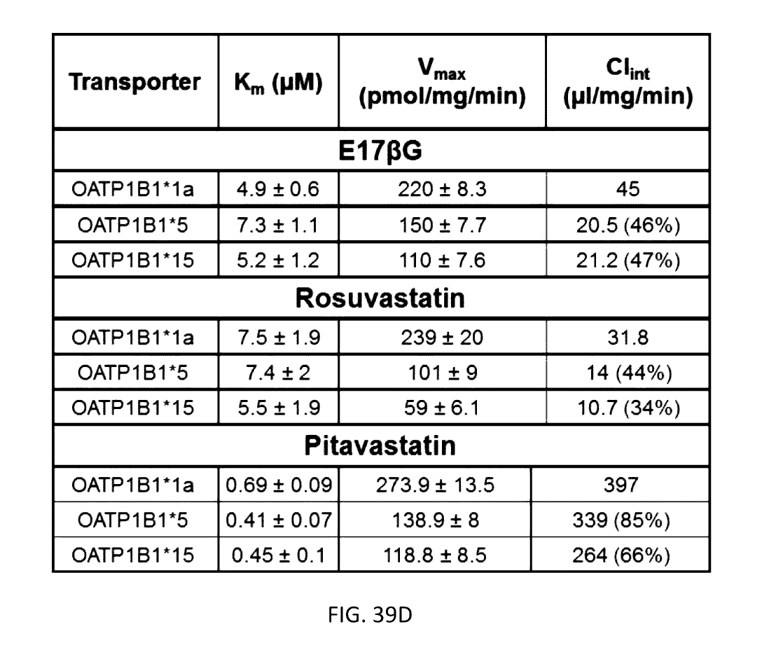

Additional assays were conducted wherein kinetic parameters (Kₘ and Vₘₐₓ) were determined in OATP1B1*1a, OATP1B1*5 and OATP1B1*15 after a 2-minute incubation at 37° C. for E17βG (1.56 μM), pitavastatin (0.2 μM) and rosuvastatin (0.78 μM); control cells were also included. For each substrate concentration, the initial uptake rate was calculated by subtracting the initial rate determined in HEK cells expressing an empty vector from those obtained in HEK-293 over-expressing SLC transporter. Each point is an average of triplicate determinations. The results are illustrated in FIG. 39A-39C; FIG. 39D illustrates the Kₘ (μM), Vₘₐₓ (pmol/mg/min), and intrinsic clearance (“CIᵢₙₜ”; μl/mg/min) calculated under Michaelis-Menten kinetics.

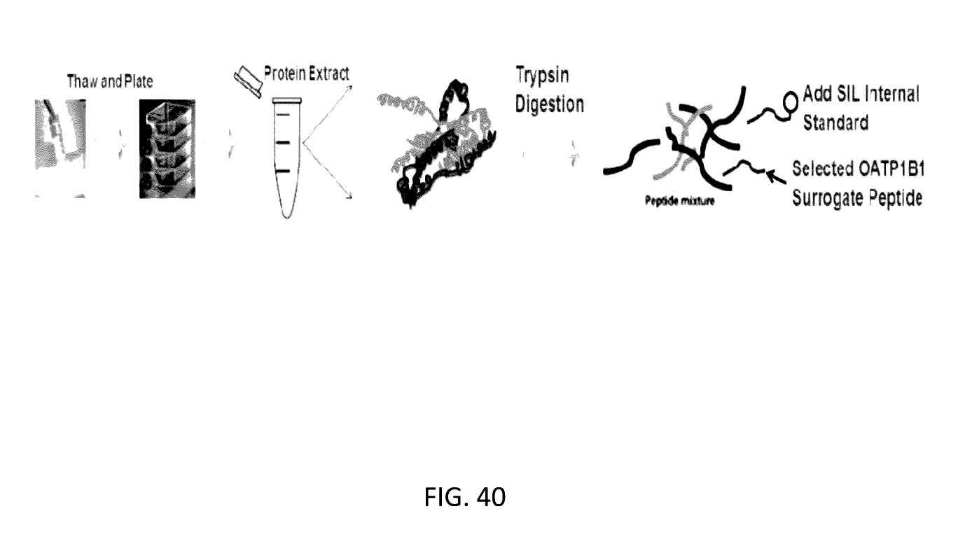

Cells were thawed and plated at the same density as the assays and re-fed with 2 mM sodium butyrate at 3 to 4 hours after plating. At 24-hours post-plating, cells were harvested, washed and then lysed using Native Membrane Protein Extraction Kit (Merck Millipore). Protein content was determined using a BCA kit (Thermo Fisher). 40 μg of protein per sample was then reduced with 10 mM DTT and alkylated with IAA in 50 mM ammonium bicarbonate digestion buffer. After adding stable isotope labeled internal standard peptide (NVTGFFQSF [KC13N15]), the samples were digested by trypsin at 37° C. for 3 hours and then at 30° C. overnight. At the end of digestion, the samples were mixed with an equal amount of 50/50 ACN/H₂O containing 0.2% formic acid and centrifuged at 3,000 rpm for 20 min prior to LC-MS/MS analysis. For standard curve, the synthetic OATP1B1 surrogate peptide (NVTGFFQSFK) was prepared in 50/50 ACN/H₂O containing 0.2% formic acid, then mixed with an equal amount of digestion matrix made from membrane extract prepared from Control Cells. LC-MS/MS was modified based on the published method (Ji C, et al., Analytica Chimica Acta (717):67-76 (2012); Wang L, et al, Drug Metab Dispos (43):367-374 (2015)). The process is graphically illustrated in FIG. 40, which is a schematic diagram of LC-MS/MS mediated targeted protein quantification.

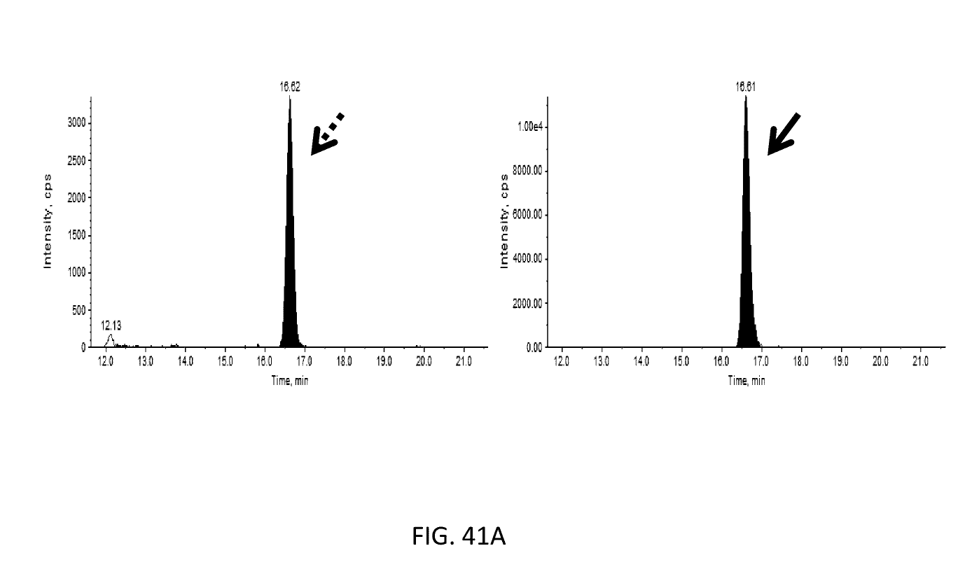

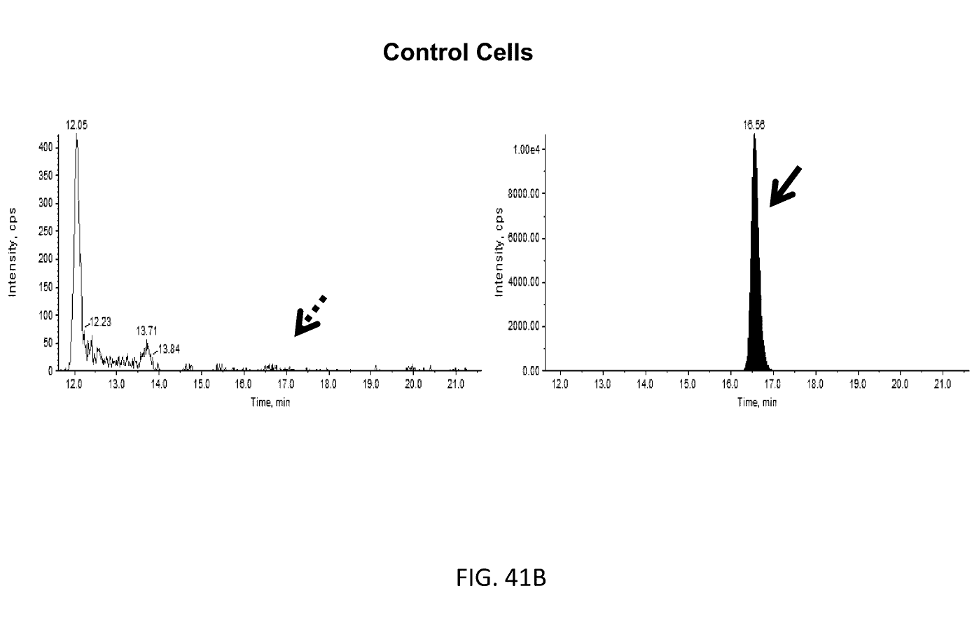

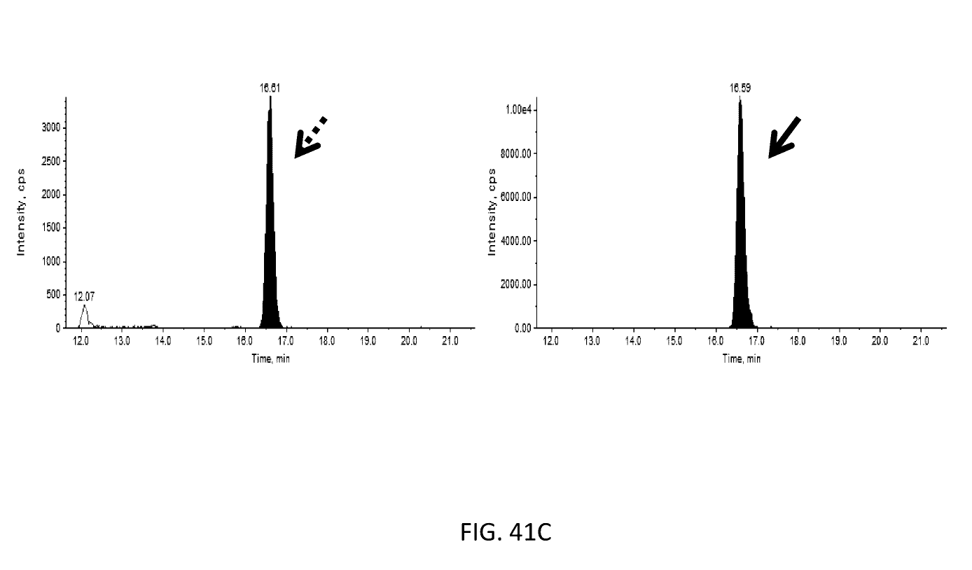

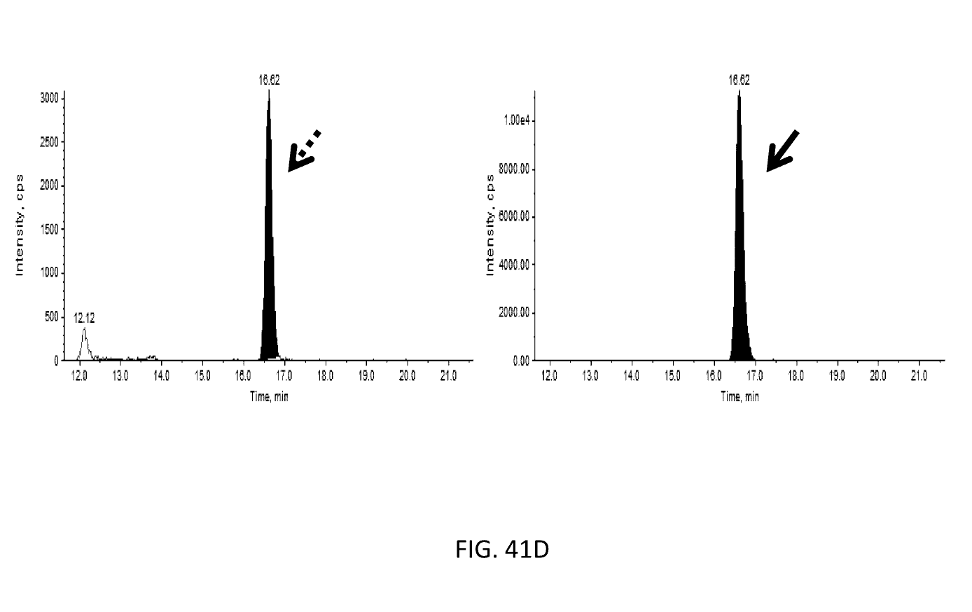

Extract ion chromatogram of selected reaction monitoring (SRM) was conducted at m/z 588.0>m/z 961.8 transition (striped arrow on FIGS. 41A-41D) for AQUA® peptide (Sigma-Aldrich) and at m/z 591.9>m/z 969.8 transition (solid arrow on FIGS. 37A-37D) for the stable isotope labeled internal standard in the tryptic digested samples from CORNING® TRANSPORTOCELLS™ OATP1B1*1a, control cells, OATP1B1*5 and OATP1B1*15; the results are illustrated in FIGS. 41A-41D (peak retention time is at RT=16.6 min).

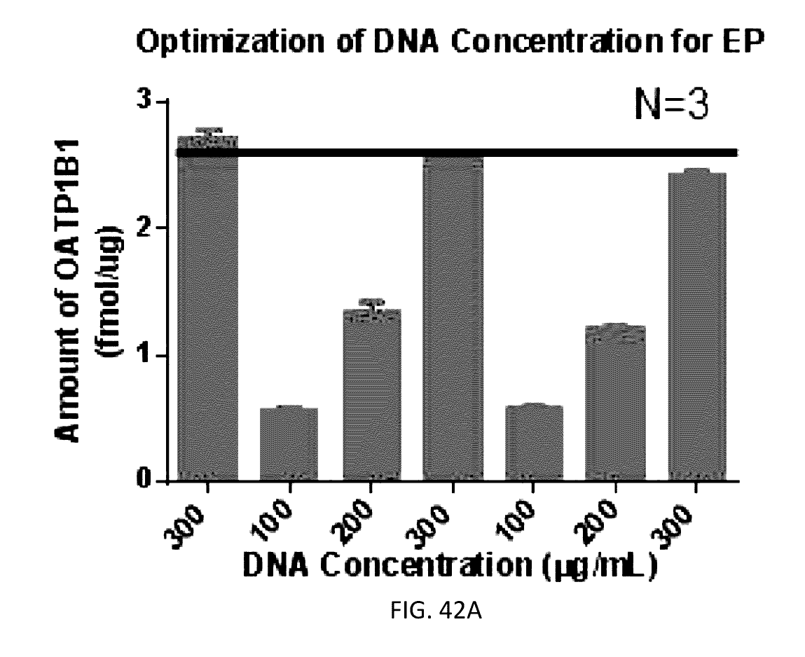

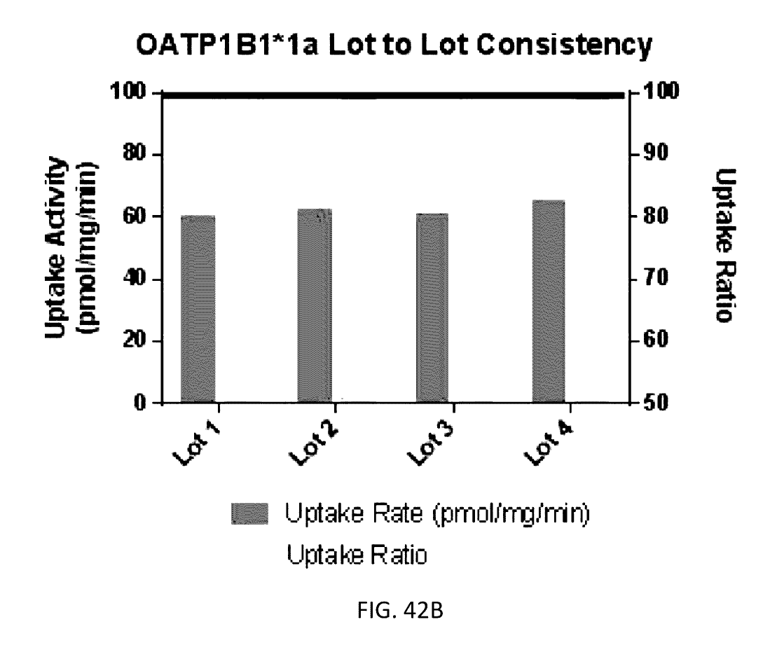

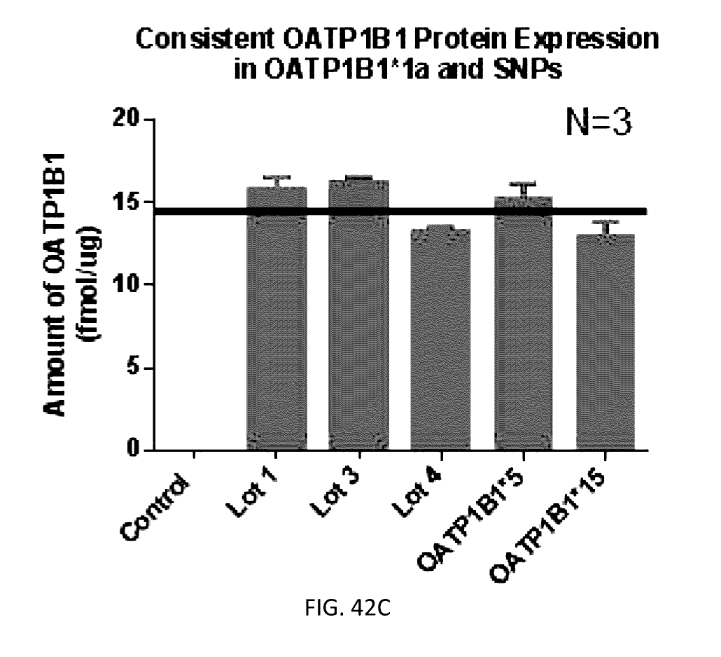

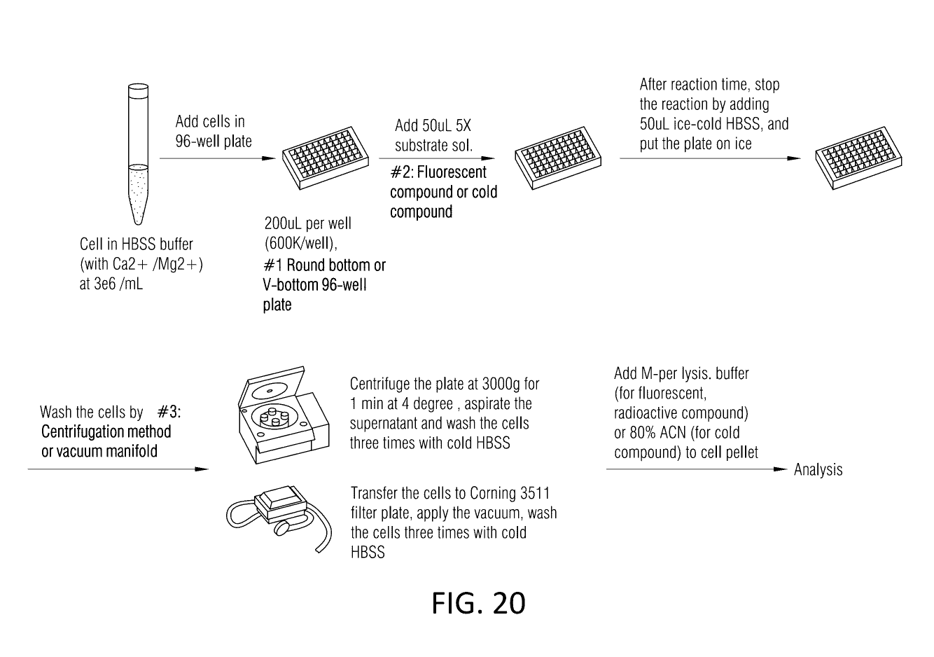

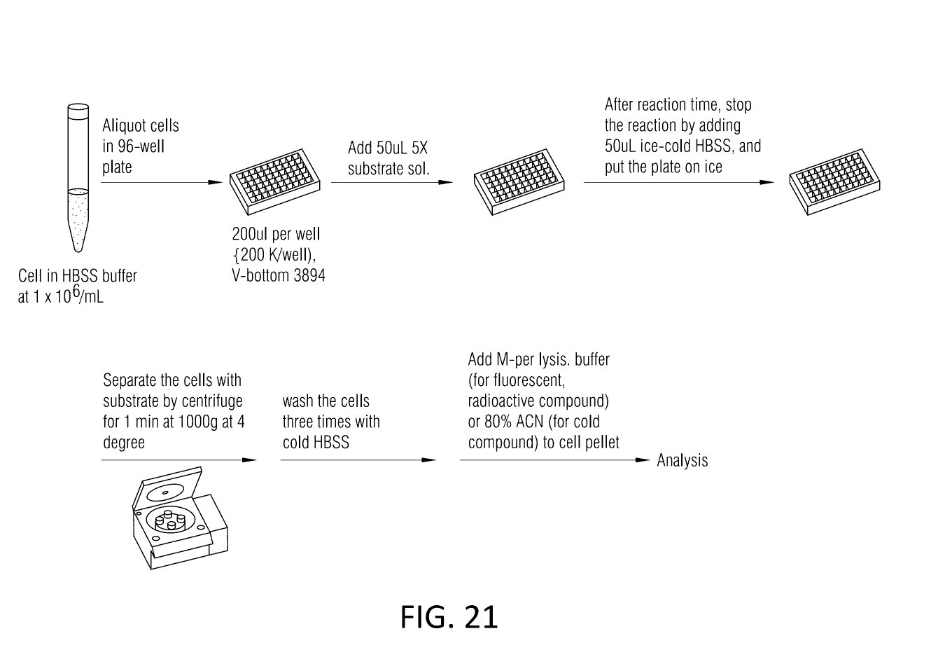

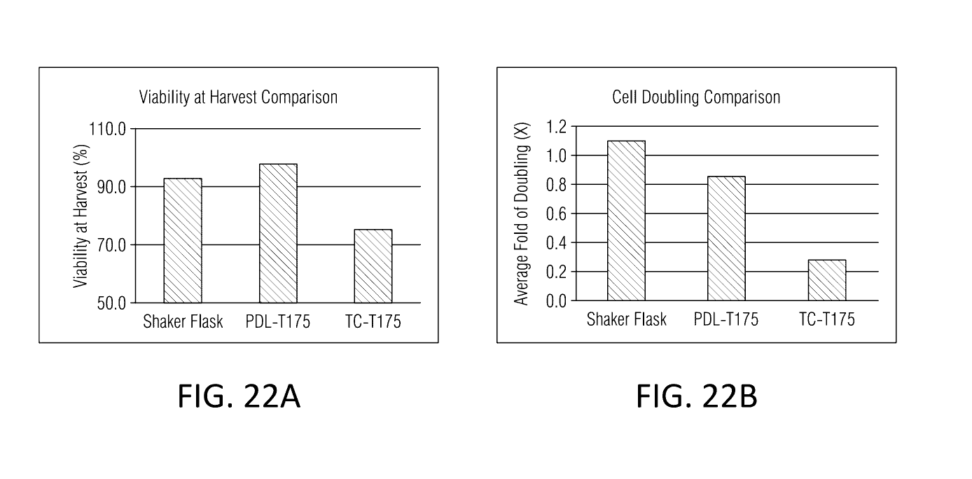

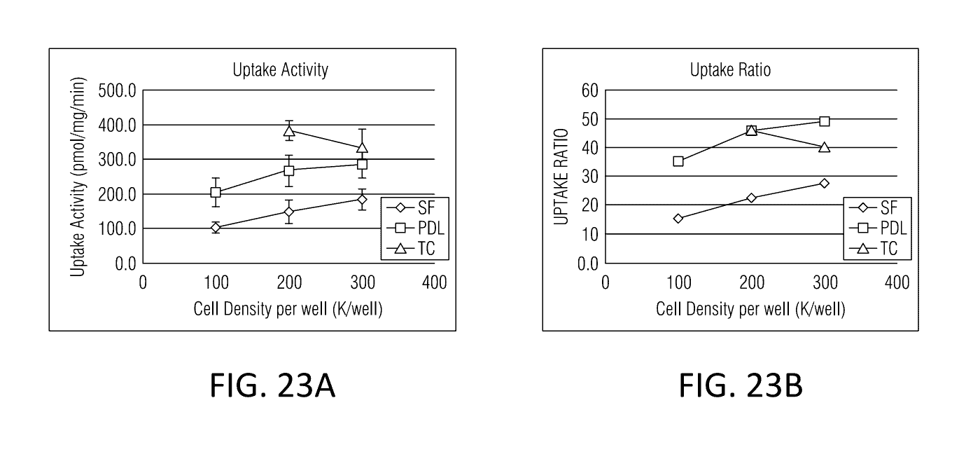

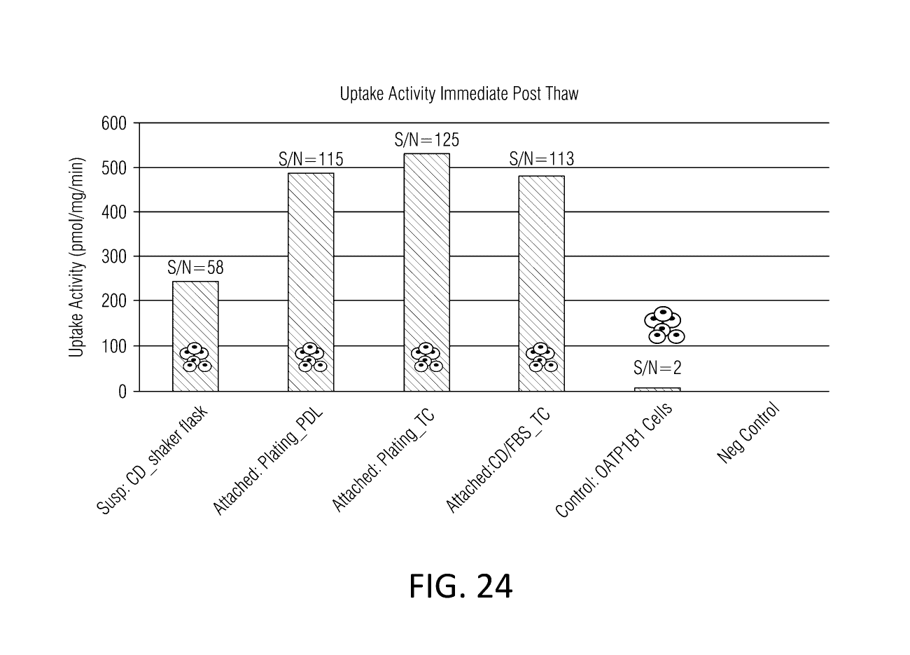

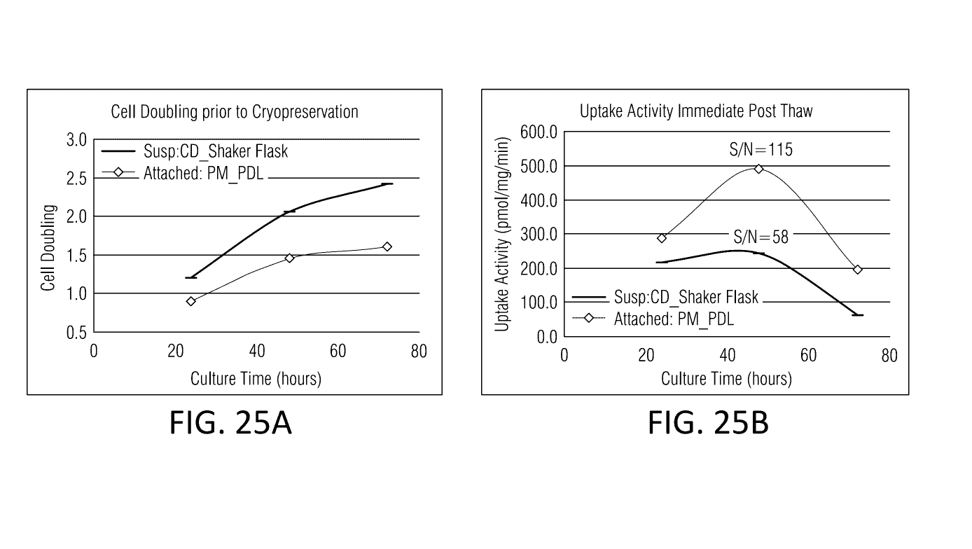

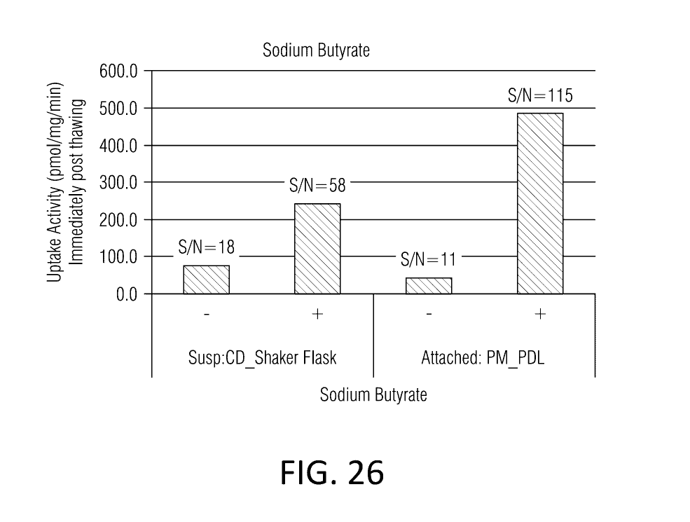

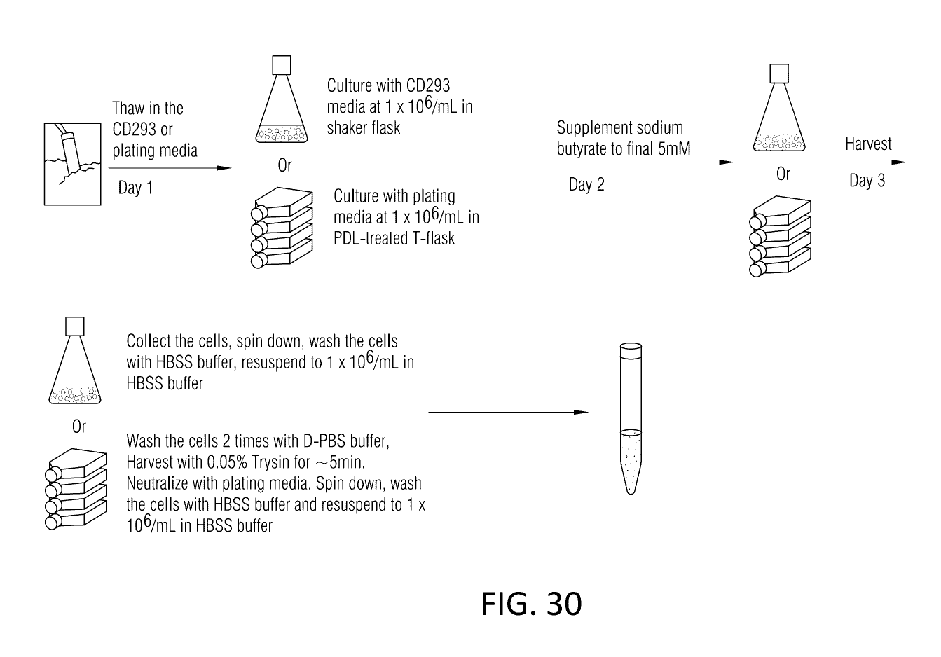

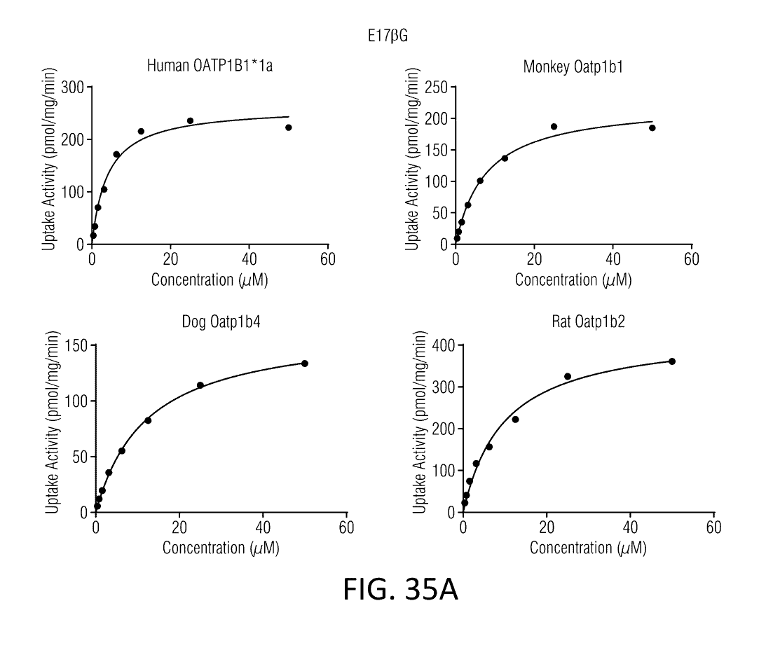

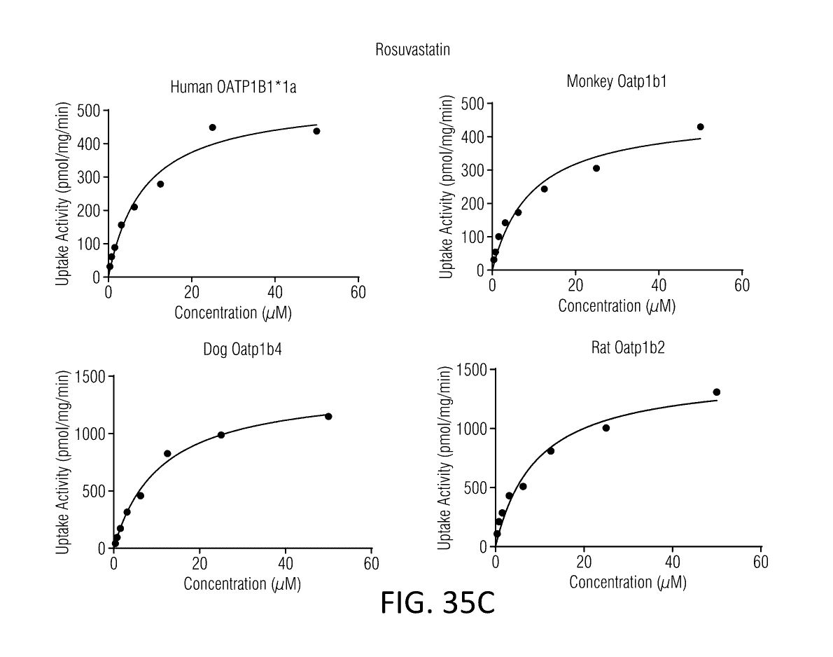

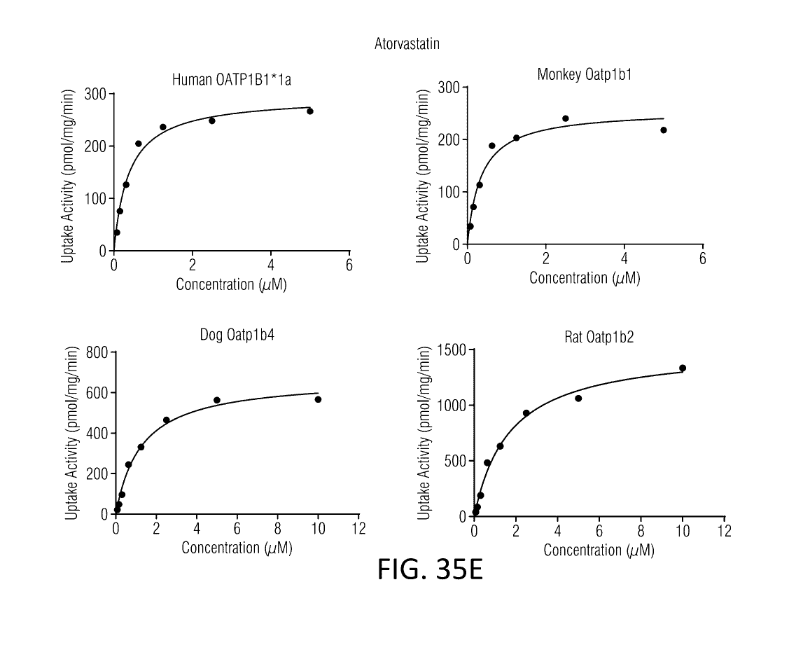

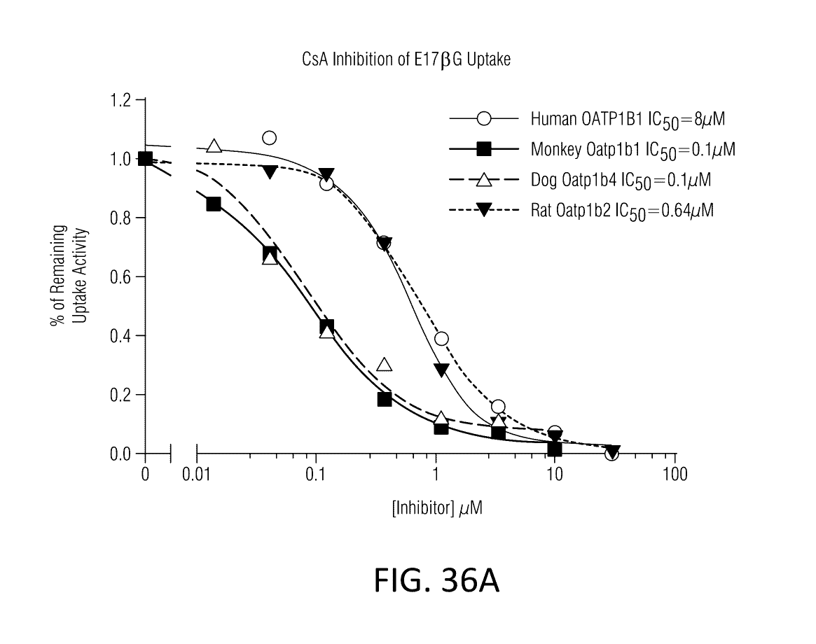

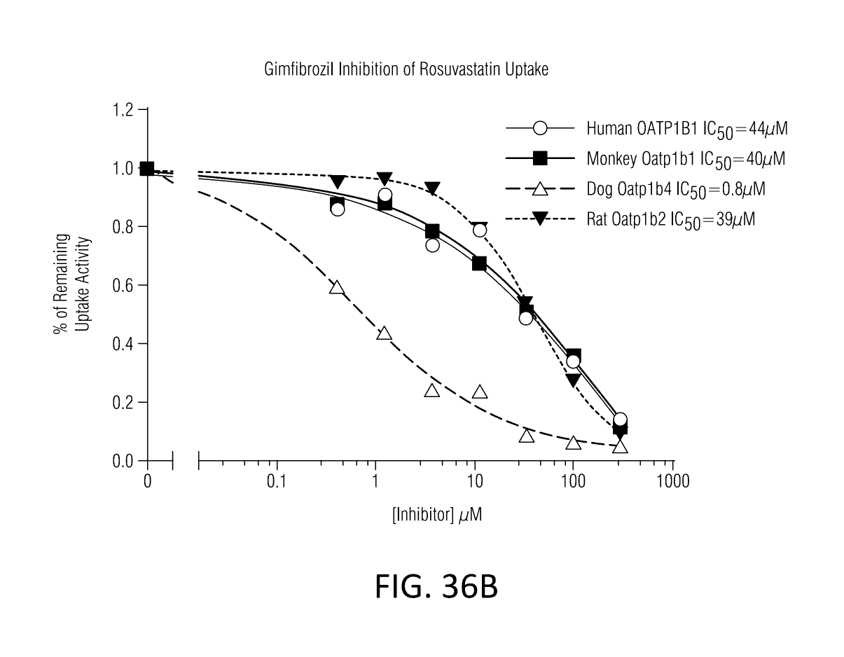

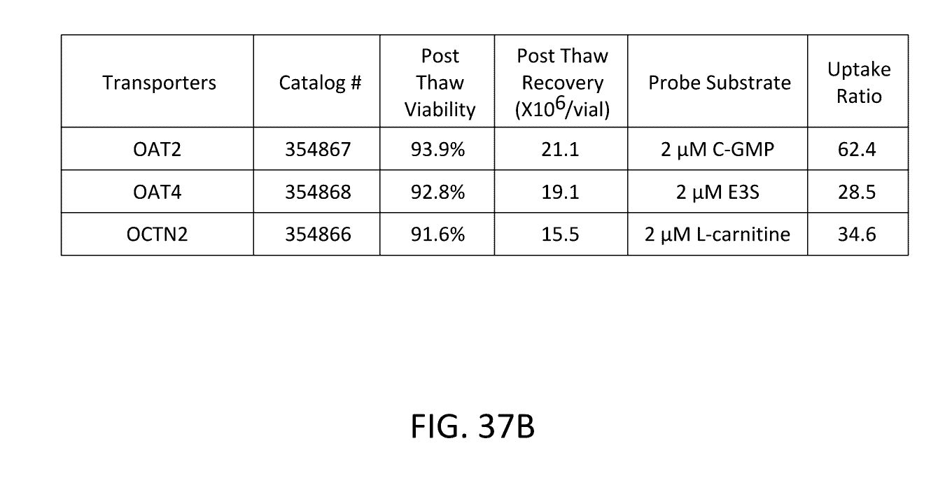

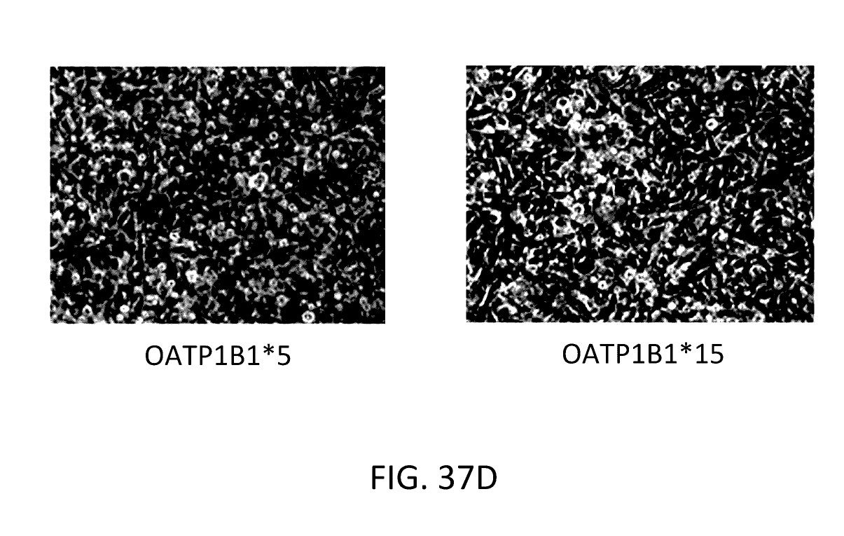

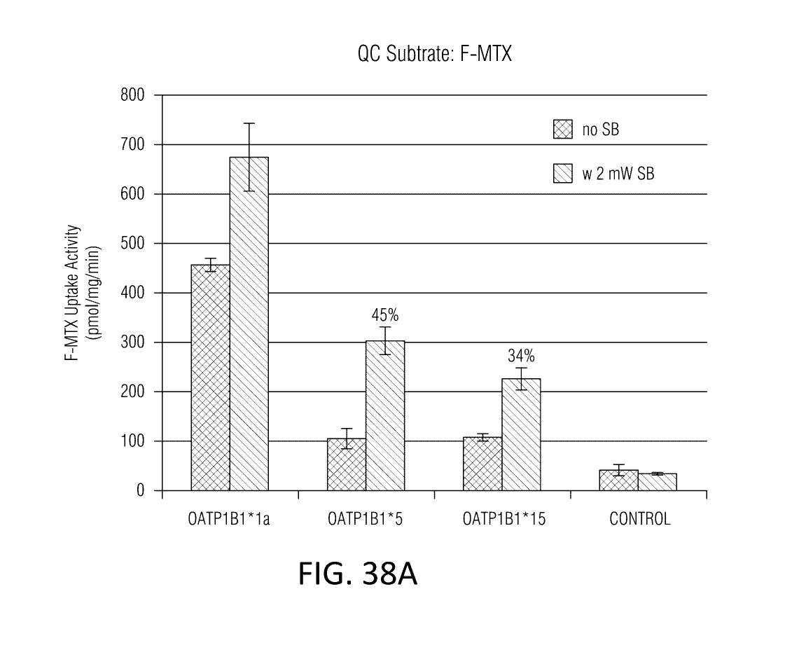

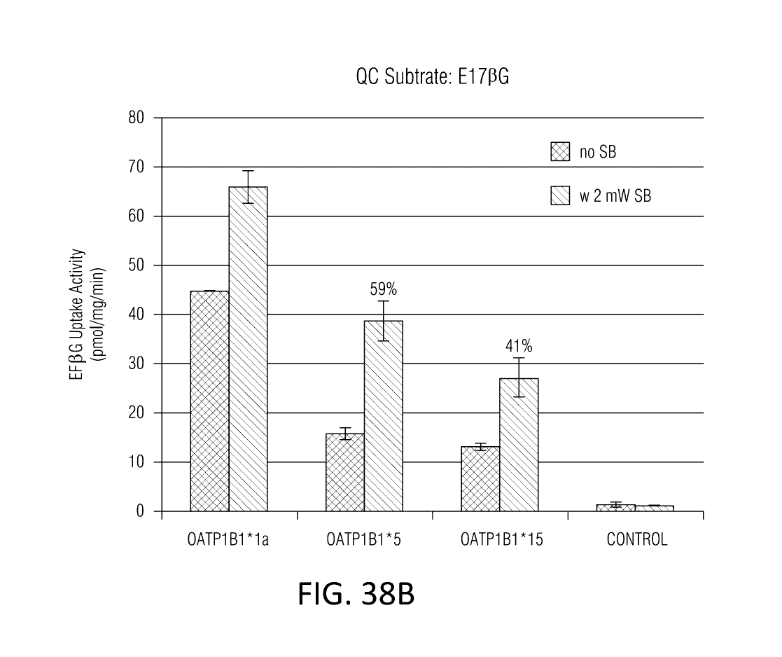

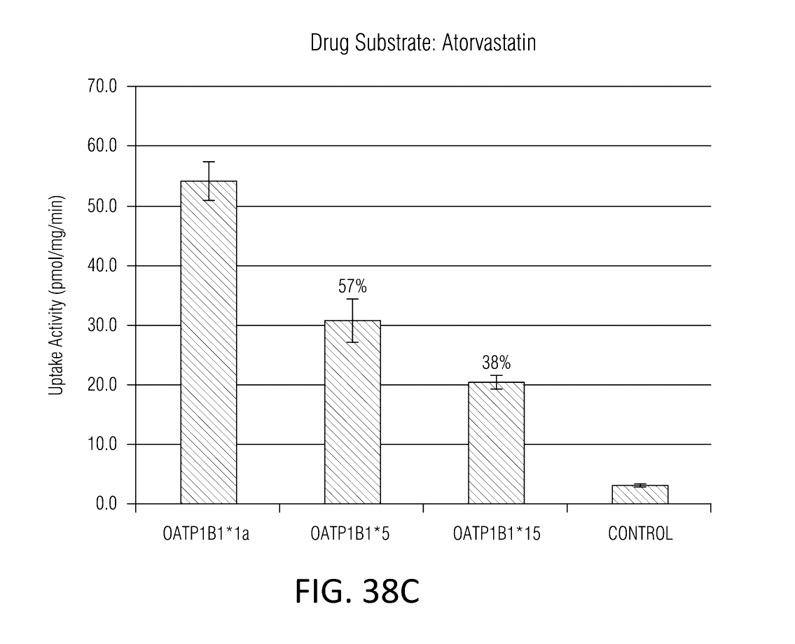

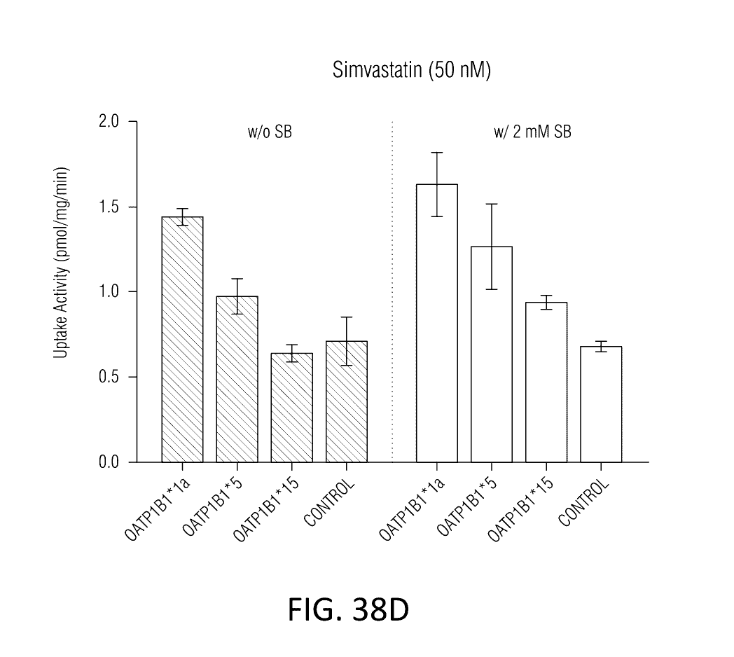

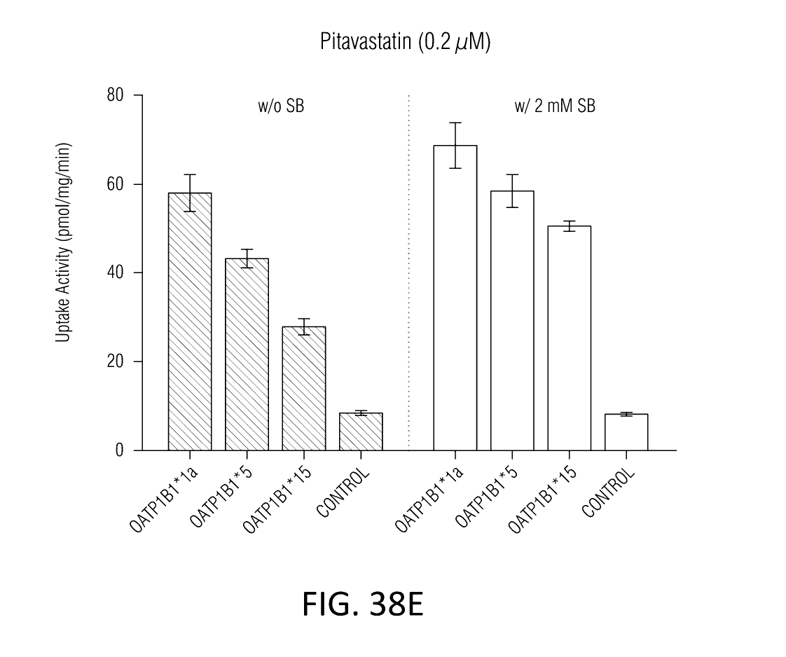

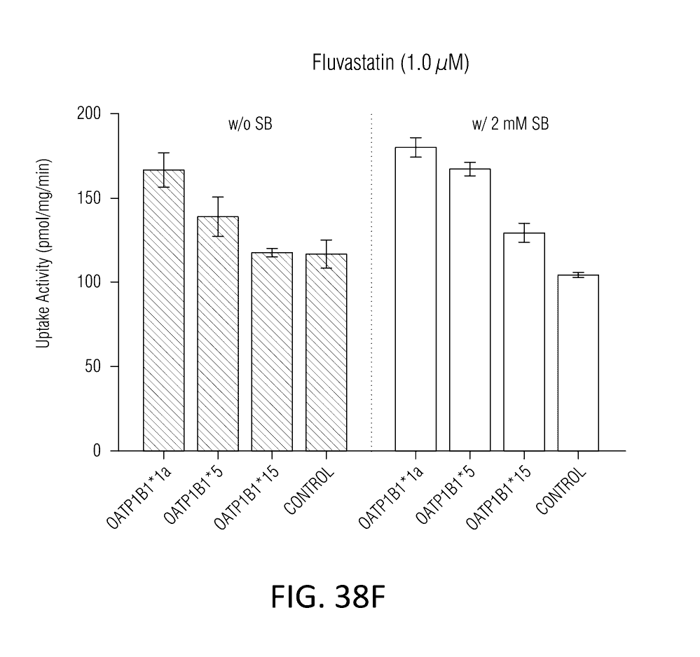

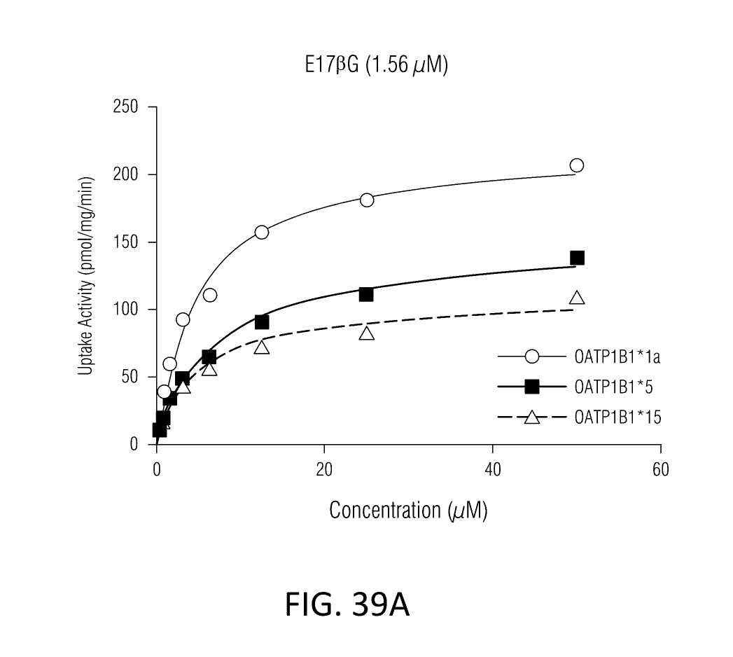

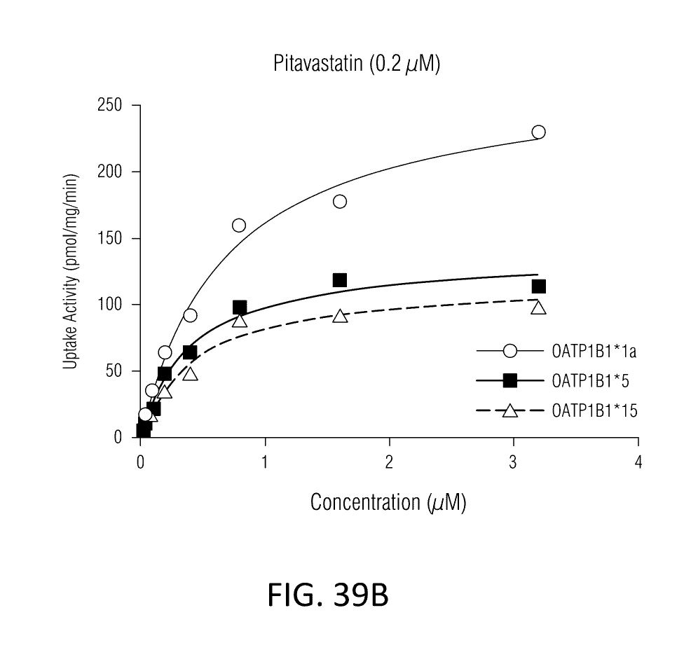

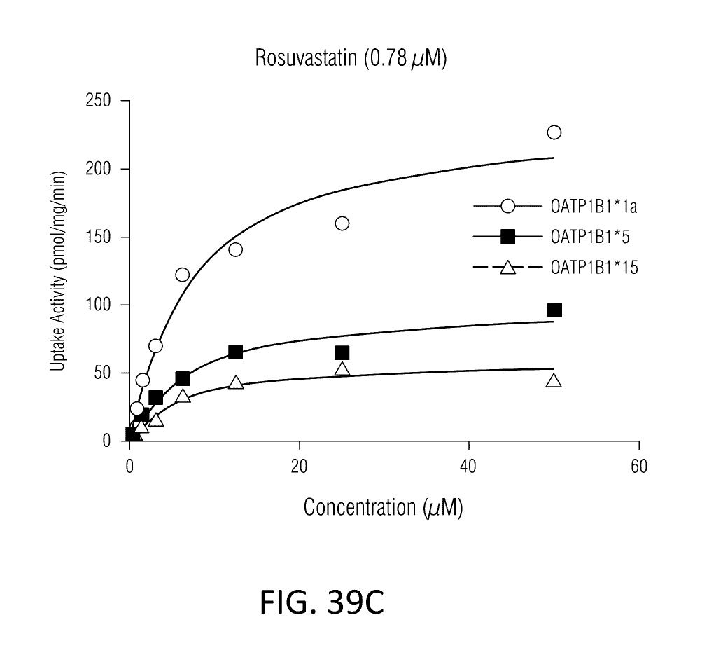

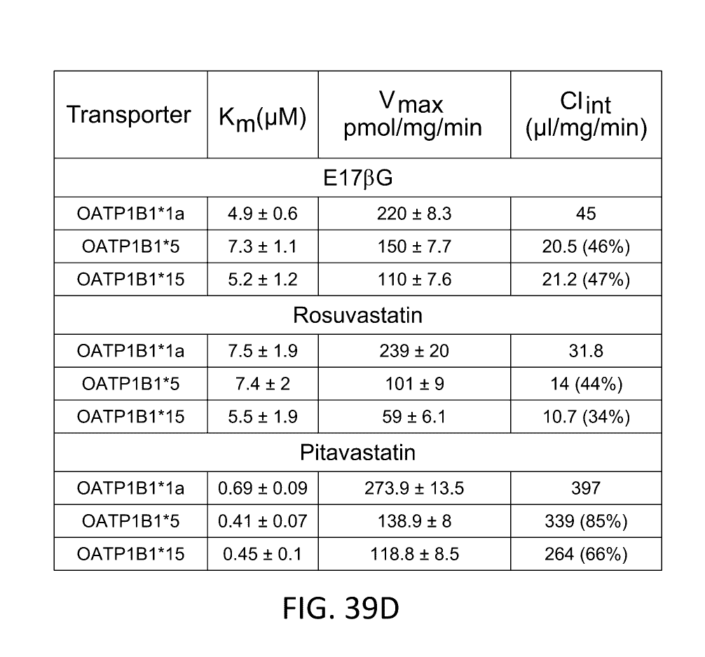

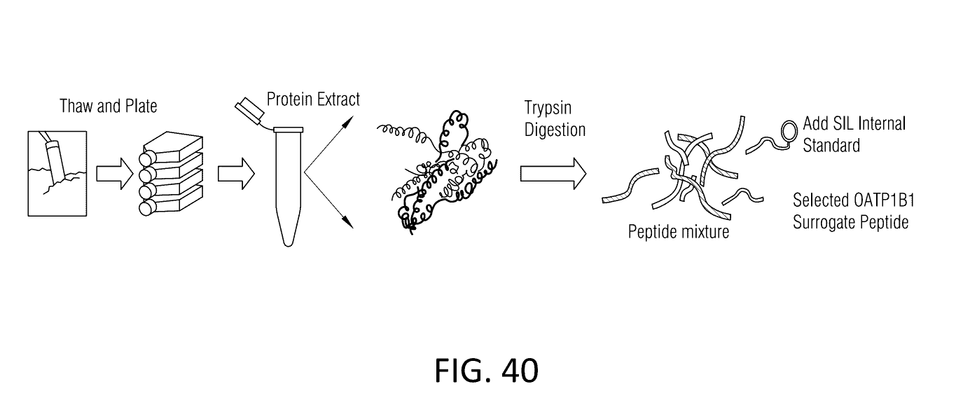

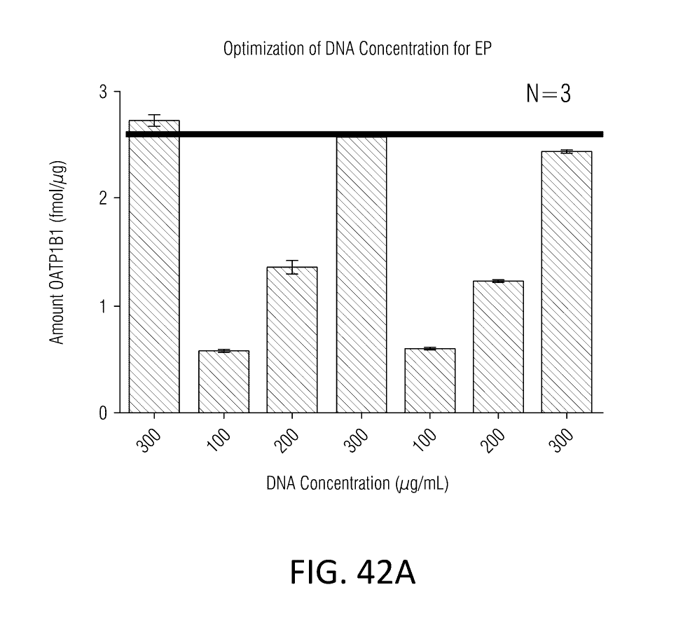

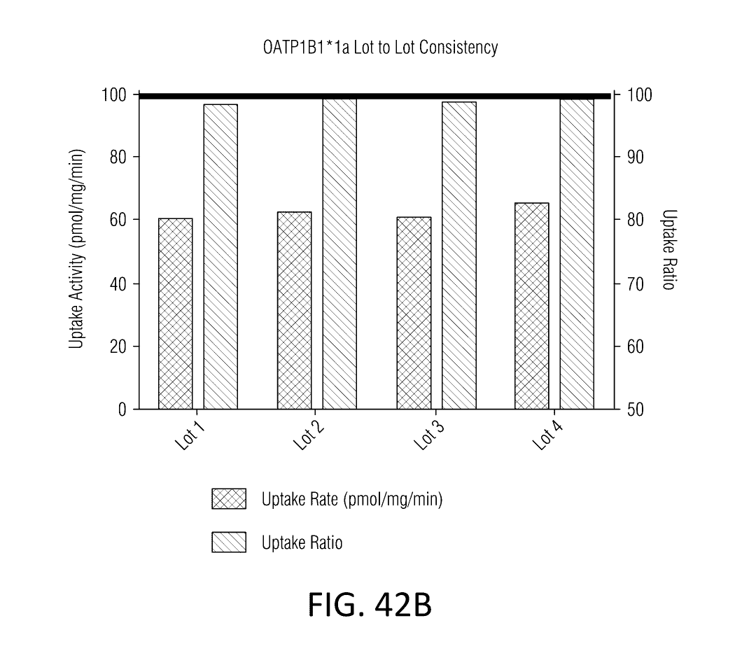

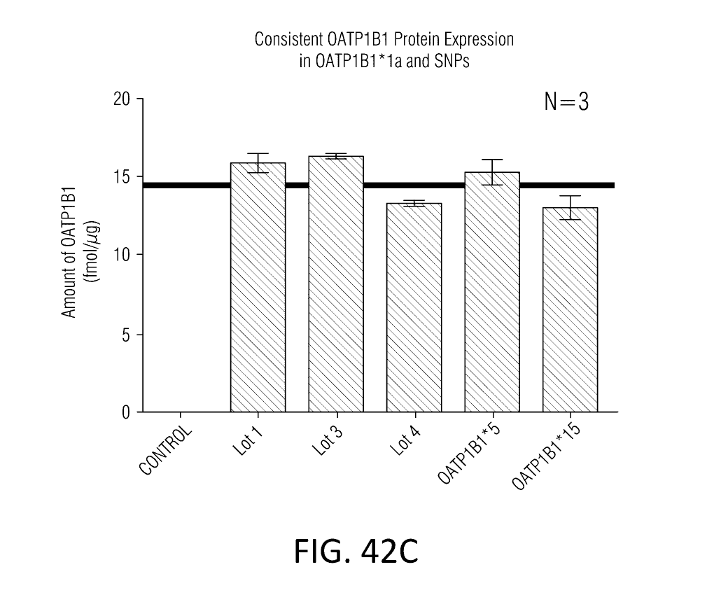

Expression in OATP1B1*5 and *15 was comparable to that of OATP1B1*1a when DNA concentrations of 300 μg/ml were reached, as illustrated in FIG. 42A. Testing proved lot-to-lot consistency. Specifically, uptake of 2 μM E17βG in both OATP1B1*1a cells and control cells was determined at 5 minutes of incubation at 37° C. Four lots of OATP1B1*1a cells were thawed and plated at the same time at 200 k per well in a Corning 38-well poly-D-lysine coated plate; the uptake activity and uptake ratios are shown in FIG. 42B. A comparison of three lots of OATP1B1*1a, one lot of OATP1B1*5, one lot of OATP1B1*15, and control cells also demonstrated consistency of protein expression across the wild-type and SNPs, as illustrated in FIG. 42C.

HEK-293 cells transiently overexpressing OATP1B1 genetic variants, i.e., OATP1B1*5, and OATP1B1*15, were developed and validated. The recombinant protein expression level in CORNING® TRANSPORTOCELLS™ OATP1B1*5 and *15 is consistent with wild-type OATP1B1*1a cells. There was no detectable OATP1B1 baseline in the parent HEK-293 cells. (3) Significantly impaired transport in OATP1B1*5 and *15 cells was observed for estradiol-17β-glucuronide, F-MTX and statins (e.g., simvastatin, pitavastatin, rosuvastatin, and fluvastatin), which is aligned with clinical findings, with the exception of fluvastatin, which does not show significant differences in clinical fluvastatin AUC between *1a and the two variant haplotypes *5 and *15. (4) CORNING® TRANSPORTOCELLS™ products evidence robust uptake ratios for all products, as well as consistent lot-to-lot uptake activity and consistent recombinant protein expression level.

### Example 3

### Development of Suspension Assay for Characterizing Activity of Drug Transporter Proteins in Corning® Transportocells™

Experimental Protocol.

In this experiment, a suspension assay for characterizing the activity of a drug transporter protein in cryopreserved, Corning® TransportoCells™ was developed. More specifically, the use of a centrifugation method versus a vacuum manifold for separating unreacted substrate in characterizing the activity of Organic Anion-Transporting Polypeptide 1B1 was investigated. Corning® TransportoCells™ transiently transfected with the gene OATP1B1*1a were obtained from Corning Life Sciences (Cat. No. 354859). In the Corning® TransportoCells™, the gene OATP1B1*1a was delivered into HEK293 cells via electroporation and the HEK293 cells were recovered and cryopreserved 1 hour post-electroporation. In order to obtain suitable expression of the Organic Anion-Transporting Polypeptide 1B1 encoded by OATP1B1*1a, the Corning® TransportoCells™ were thawed, cultured, and harvested.

More specifically, the Corning® TransportoCells™ were thawed in a water bath at 37° C. for about 2 minutes, pelleted down by spinning at 100 g for 5 minutes, and the cell pellet was resuspended in appropriate amount of plating media (detailed in Table 16) at a cell density of 1×10⁶ cells/ml. The cells were cultured in T-175 Vented-Cap Culture Flasks with Poly-D-Lysine (hereinafter, “PDL”; available from Corning Life Sciences, Cat. No. 354539) with plating medium for 48 hours at 37° C. and 8% CO₂. The plating medium is detailed in Table 16. After 24 hours, sodium butyrate (obtained from Sigma) was added to the cells to reach final 5 mM. After 48 hours, the cells were rinsed twice with Phosphate-Buffered Saline (hereinafter, “PBS”, obtained from Corning).

TABLE 16
Plating Media*

| Reagent | Quantity |
|---|---|
| Dulbecco's Modified Eagle Medium | 445 mL |
| (hereinafter, “DMEM”) with high glucose | |
| (available from Gibco, Cat. No. 11965092, Life | |
| Technologies Corp.) | |
| MEM Non-Essential Amino Acid Solution | 5 mL |
| (100X) (available from Gibco, Cat. No. | |
| 11140050, Life Technologies Corp.) | |
| Fetal Bovine Serum (hereinafter, “FBS”; | 50 mL |
| available from SAFC Biosciences, Cat. No. | |
| 12016C, Sigma) | |

*Plating media was sterilized using a 0.2 μm filter and storing at 4° C. for up to 2 weeks.

The cells were then harvested with 0.05% Trypsin (obtained from Sigma) and washed once with Hank's Balanced Salt Solution (hereinafter, “HBSS”) buffer (with Ca²⁺ and Mg²⁺, obtained from Corning). Then, the cells were resuspended in HBSS (obtained from Corning) to a final cell density of 3×10⁶ cells/ml. Two suspension assay experiments were then performed to characterize the activity of Organic Anion-Transporting Polypeptide 1B1 in the Corning® TransportoCells™.

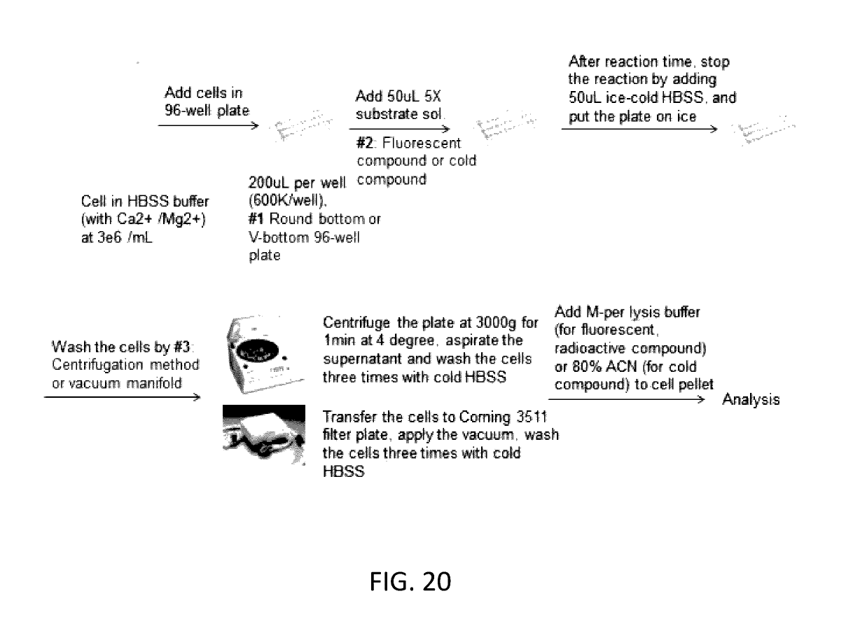

In a first suspension assay experiment, use of a centrifugation method was investigated to separate excess substrate and cells. Referencing FIG. 20, in the first suspension assay experiment, the resuspended Corning® TransportoCells™ were aliquotted (200 μl per well at a density of 600 k cells/well) into either a Corning® 96 Well Clear Round Bottom TC-Treated Microplate (available from Corning Life Sciences, Cat. No. 3799) or a Corning® 96 Well Clear V-Bottom TC-Treated Microplate (available from Corning Life Sciences, Cat. No. 3894). The activity of Organic Anion-Transporting Polypeptide 1B1 was characterized by initiating a reaction by adding either prewarmed 50 μl 5× substrate solution (50 μl HBSS buffer containing 25 μM Estradiol 17-β Glucuronide, hereinafter, “E17βG”; obtained from Sigma) or by adding fluorescent 50 μl 5× substrate solution (50 μl HBSS buffer containing either 25 μM fluorescein methotrexate, hereinafter, “FMTX”; obtained from Life Technologies or 25 μM 8-fluorescein-cAMP, hereinafter, “8-FcA”; obtained from BIOLOG Life Sciences). The cells were then incubated for 10 minutes at 37° C. After the incubation time, the reaction was stopped by adding ice cold HBSS buffer (50 μl) to the cells and placing the microplates on ice. Then, the microplates were centrifuged at 3000 g for 1 minute at 4° C. The supernatant was aspirated and the cells were washed three times with 200 uL cold HBSS. The cells contacted with the non radioactive substrate solution containing E17βG were lysed with 80% Acetonitrile lysis buffer (made inhouse). The cells contacted with the fluorescent substrate solution containing FMTX or 8-FcA were lysed with M-per protein lysis buffer (200 μL, obtained from Thermo Scientific). The cell lysis was then subjected to the appropriate protein analysis and/or fluorescence analysis to characterize the activity of Organic Anion-Transporting Polypeptide 1B1.

In a second suspension assay experiment, use of a vacuum manifold was investigated. Referencing FIG. 20, in the second suspension assay experiment, the resuspended Corning® TransportoCells™ were aliquotted (200 μl per well at a density of 600 k cells/well) into a Corning® 96 Well Clear Round Bottom TC-Treated Microplate (available from Corning Life Sciences, Cat. No. 3799). The activity of Organic Anion-Transporting Polypeptide 1B1 was characterized by initiating a reaction by adding either prewarmed 50 μl 5× substrate solution (50 μl HBSS buffer containing 25 μM E17βG; obtained from Sigma) or by adding 50 μl fluorescent 5× substrate solution (50 μl HBSS buffer containing 25 μM FMTX; obtained from Life Technologes; or 25 μM 8-FcA; obtained from BIOLOG Life Sciences). The cells were then incubated for 10 minutes at 37° C. After the incubation time, the reaction was stopped by adding ice cold 50 μl HBSS buffer and placing the microplates on ice.

Then, the cells were transferred to FiltrEX™ 96 Well Filter Plates with 0.66 mm Thick Glass Fiber Filter (available from Corning Life Sciences, Cat. No. 3511) and a vacuum was applied. In this method, substrate solution flows through the filter plate and is collected in the receiver plate while insoluble particles, such as, e.g., membrane vesicles or cells, are trapped on the filter plate. The cells trapped on the filter plate were washed three times with cold HBSS. The cells contacted with the non radioactive substrate solution containing E17βG were lysed with 80% Acetonitrile lysis buffer (made inhouse). The cells contacted with the fluorescent substrate solution containing FMTX or 8-FcA were lysed with M-per protein lysis buffer (200 μL, obtained from Thermo Scientific). The cell lysis was collected into a new receiver plate by vacuum. The cell lysis was then subjected to the appropriate protein analysis and/or fluorescence analysis to characterize the activity of Organic Anion-Transporting Polypeptide 1B1.

A positive control was provided via an adherent assay for characterizing the activity of a drug transporter protein in cryopreserved, Corning® TransportoCells™. Corning® TransportoCells™ transiently transfected with the gene OATP1B1*1a were obtained from Corning Life Sciences (Cat. No. 354859). In order to obtain suitable expression of the Organic Anion-Transporting Polypeptide 1B1 encoded by OATP1B1*1a, the Corning® TransportoCells™ were thawed, cultured, and harvested. More specifically, the Corning® TransportoCells™ were thawed in a water bath at 37° C. for about 2 minutes, pelleted down by spinning at 100 g for 5 minutes, and the cell pellet was resuspended in plating media (obtained from Table 16) at a cell density of 1×10⁶ cells/ml. The cells were cultured via plating in a 24-well PDL-Treated Plate (cell density of 250K cells/well; obtained from Corning Life Sciences) with plating medium for 48 hours at 37° C. and 8% CO₂. The plating medium is detailed in Table 16. After 24 hours, cells were refed by 400 uL plating media supplemented with 5 mM sodium butyrate (obtained from Sigma). After 48 hours, cells were washed three times using 0.4 mL prewarmed HBSS (Corning). Then 0.3 mL substrate solution containing 5 uM FMTX were added to the cells and the cells were incubated for 10 min at 37 degree. After 10 min incubation time, the cells were washed 3 times using 0.4 mL cold HBSS. The cells were lysed and subjected to the BCA protein analysis and/or fluorescence analysis to characterize the activity of Organic Anion-Transporting Polypeptide 1B1.

Results.

As set forth in Table 17 below, cells incubated in the Corning® 96 Well Clear V-Bottom TC-Treated Microplate in the centrifugation method exhibited the highest uptake ratio (i.e., S/N=102) in the first suspension assay experiment. As also shown in Table 17, cells incubated in the Corning® 96 Well Clear V-Bottom TC-Treated Microplate in the centrifugation method of the first suspension assay experiment exhibited favorable well to well variation, i.e., CV, (n=6, CV<15%) with the fluorescent substrate FMTX. Without being bound by the theory, it is believed that a decrease in the uptake ratio and an increase in CV in the centrifugation method of the first suspension assay exhibited by cells incubated in the Corning® 96 Well Clear Round Bottom TC-Treated Microplate was due to difficulty in forming a tight congregated cell pellet during centrifugation and/or to pellet loss during washing.

TABLE 17
Uptake Activity and Uptake Ratio Comparison

| | | | Uptake Activity (pmol/mg/min) | | | | Up- |
|---|---|---|---|---|---|---|---|
| | Sub- | | | CV- | Con- | CV- | take |
| Conditions | strate | n | OATP1B1 | 1B1 | trol | Cont | Ratio |
| Centrifugation, | FMTX | 6 | 1.04 | 13% | 0.01 | 7% | 102.3 |
| V-Bottom | 8-FcA | 6 | 0.20 | 7% | 0.00 | 14% | 66.6 |
| Centrifugation, | FMTX | 6 | 0.38 | 56% | 0.03 | 121% | 10.9 |
| Round Bottom | | | | | | | |
| Vacuum | FMTX | 6 | 0.46 | 32% | 0.07 | 73% | 6.8 |
| Manifold | | | | | | | |
| PC: Plated | FMTX | 3 | 1.06 | 3% | 0.03 | 5% | 30.6 |
| Assay, Seeded | | | | | | | |
| at 250k/well | | | | | | | |
| (24-well), | | | | | | | |
| assay at 48 | | | | | | | |
| hours | | | | | | | |

Additionally, as shown in Table 18 below, the addition of the substrate solution containing non-radioactive E17βG versus the substrate solution containing fluorescent FMTX to cells in the vacuum manifold of the second suspension assay experiment exhibited similar uptake ratios. Without being bound by the theory, it is believed that the substrate was not trapped on the filter plate in vacuum manifold of the second suspension assay experiment, which means, the low uptake ratio with vacuum manifold is not due to substrate trapped on the filter plate, but due to the different way of separating unreacted substrate with the cells.

TABLE 18
Uptake Activity and Uptake Ratio Comparison of Vacuum Manifold
Protocol with FMTX and E17βG

| | | | Uptake Activity (pmol/mg/min) | | | | |
|---|---|---|---|---|---|---|---|
| | Sub- | | | CV- | Con- | CV- | Uptake |
| Conditions | strate | n | OATP1B1 | 1B1 | trol | Cont | Ratio |
| Vacuum | FMTX | 6 | 0.46 | 32% | 0.07 | 73% | 6.8 |
| Manifold with | | | | | | | |
| Fluorescent | | | | | | | |
| Compound | | | | | | | |
| Vacuum | E17βG | 6 | 2.19 | 65% | 0.35 | 70% | 6.4 |
| manifold with | | | | | | | |
| non- | | | | | | | |
| radioactive | | | | | | | |
| Compound | | | | | | | |

An appropriate suspension assay protocol employing the centrifugation method is depicted in FIG. 21.

### Example 4

### Characterization of Culturing Conditions and Cell Density for Cryopreserved, Transiently Transfected HEK 293 Cells

Experimental Protocol.

In this experiment, a suspension assay for characterizing the activity of a drug transporter protein in cryopreserved, Corning® TransportoCells™ was further developed. More specifically, the effect of culturing conditions and cell density per well in the assay on the activity of Organic Anion-Transporting Polypeptide 1B1 in Corning® TransportoCells™ was investigated. With regard to culturing conditions, the effect of shaker flask culturing, and T-flask culturing on the activity of Organic Anion-Transporting Polypeptide 1B1 in Corning® TransportoCells™ was investigated. Corning® TransportoCells™ transiently transfected with the gene OATP1B1*1a were obtained from Corning Life Sciences (Cat. No. 354859). With regard to cell density, the effect of cell density in the Suspension Assay on the activity of Organic Anion-Transporting Polypeptide 1B1 in Corning® TransportoCells™ was investigated. In order to obtain suitable expression of the Organic Anion-Transporting Polypeptide 1B1 encoded by OATP1B1*1a, the Corning® TransportoCells™ were thawed, cultured via shaker flask culturing, or T-flask culturing, and harvested. More specifically, the Corning® TransportoCells™ were thawed in a water bath at 37° C. for about 2 minutes, pelleted down by spinning at 100 g for 5 minutes, and the cell pellet was resuspended in plating media (obtained from Table 16) at a cell density of 1×10⁶ cells/ml.

In a first shaker flask culturing experiment, the cells were cultured in Erlenmeyer shaker flasks (obtained from Corning) with CD293 media (obtained from Life Technologies) for 48 hours at 37° C. and 8% CO₂ and with shaking at 100 RPM. After 24 hours, sodium butyrate (to a final concentration of 5 mM, obtained from Sigma) was added to the cells. After 48 hours, the cells were harvested via centrifugation. Cell viability and cell number were determined, as previously described. The cells were resuspended after centrifugation in HBSS (obtained from Corning) and aliquotted into a Corning® 96 Well Clear V-Bottom TC-Treated Microplate (obtained from Corning Life Sciences, Cat. No. 3894) to a final cell density of 100K cells/well, 200K cells/well, or 300K cells/well. The cells were then assayed in the microplate to characterize the activity of Organic Anion-Transporting Polypeptide 1B1 following the centrifugation method described in Example 2.

In a second T-flask culturing experiment, the cells were cultured in either Corning® BioCoat™ PDL 175 cm² Rectangular Straight Neck Cell Culture Flasks with Vented Cap (available from Corning Life Sciences, Cat. No. 354539) or in Falcon® 175 cm² Rectangular Straight Neck Cell Culture Flasks with Vented Cap (available from Corning life Sciences, Cat. No. 353112) via plating in attached form. The Falcon® 175 cm² Rectangular Straight Neck Cell Culture Flasks with Vented Cap were TC-treated. The cells were cultured with plating medium for 48 hours at 37° C. and 8% CO₂. The plating medium is detailed in Table 16. After 24 hours, sodium butyrate (to a final concentration of 5 mM, (obtained from Sigma) was added to the cells. After 48 hours, the cells were rinsed twice with PBS (obtained from Corning). The cells cultured in the BioCoat™ PDL 175 cm² Rectangular Straight Neck Cell Culture Flasks with Vented Cap were harvested with 0.05% Trypsin (obtained from Sigma). Cell viability and cell number were determined, as previously described. The cells were then washed once with HBSS buffer (with Ca²⁺ and Mg²⁺, obtained from Corning). The cells cultured in the Falcon® 175 cm² Rectangular Straight Neck Cell Culture Flasks with Vented Cap were harvested with Dulbecco's PBS (hereinafter, “D-PBS”; obtained from Corning). Cell viability and cell number were determined, as previously described. The cells were then washed once with HBSS buffer (with Ca²⁺ and Mg²⁺, obtained from Corning). The cells were resuspended after harvesting in appropriate volume of HBSS to reach 1×10⁶ cells/ml, and aliquotted into a Corning® 96 Well Clear V-Bottom TC-Treated Microplate (obtained from Corning Life Sciences, Cat. No. 3894) to a final cell density of 100K cells/well, 200K cells/well, or 300K cells/well. The cells were then assayed in the microplate to characterize the activity of Organic Anion-Transporting Polypeptide 1B1 following the centrifugation method described in Example 2.

Results.

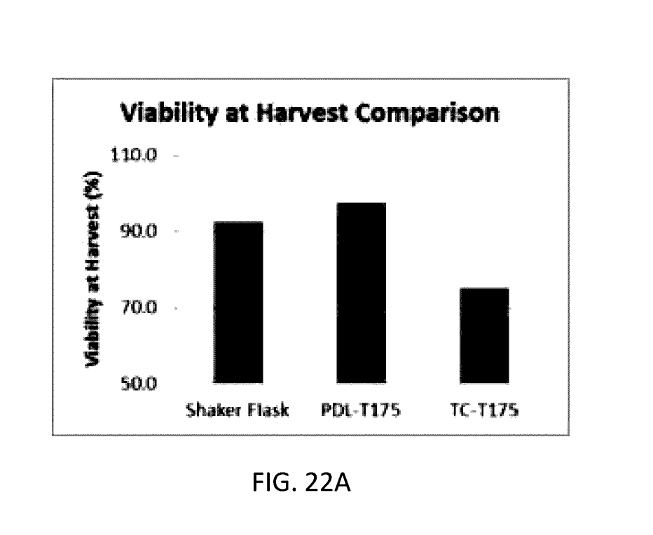

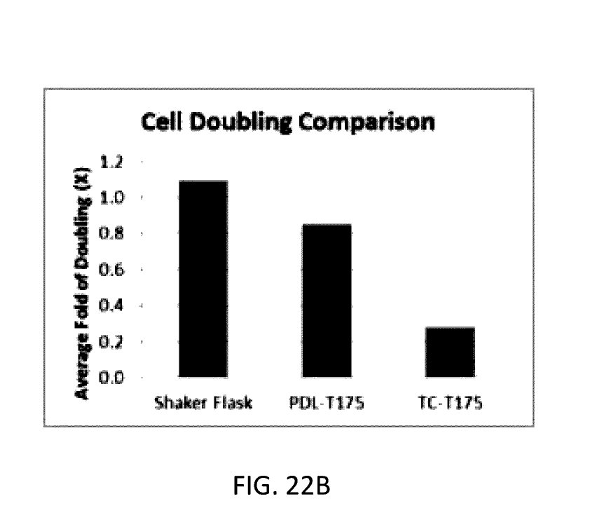

As shown in FIG. 22A, the cells cultured in the Erlenmeyer shaker flasks in the first shaker flask culturing experiment and the cells cultured in the Corning® BioCoat™ PDL 175 cm² Rectangular Straight Neck Cell Culture Flasks with Vented Cap in the second T-flask culturing experiment both exhibited high viability of 90% and good cell recovery at harvest. Further, as shown in FIG. 22B, the cells cultured in the Erlenmeyer shaker flasks exhibited higher cell doubling as compared to the cells cultured in the Corning® BioCoat™ PDL 175 cm² Rectangular Straight Neck Cell Culture Flasks with Vented Cap. Without being bound by the theory, it is believed that the higher cell doubling of the Erlenmeyer shaker flasks as compared to the cells cultured in the Corning® BioCoat™ PDL 175 cm² Rectangular Straight Neck Cell Culture Flasks with Vented Cap was likely due to minimizing cell loss at harvest. Cell doubling was calculated based on cell recovery after harvest and washing divided by the initial amount of cells added.

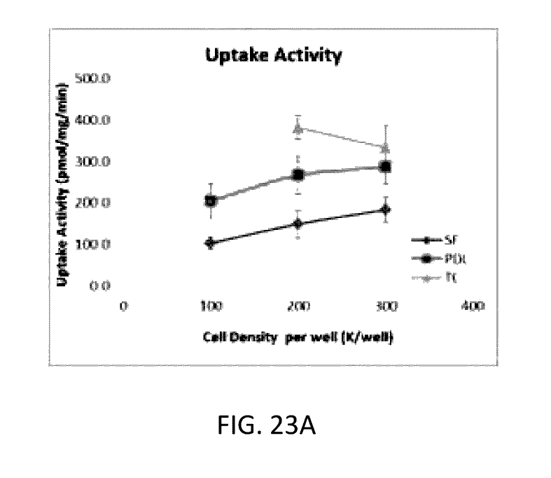

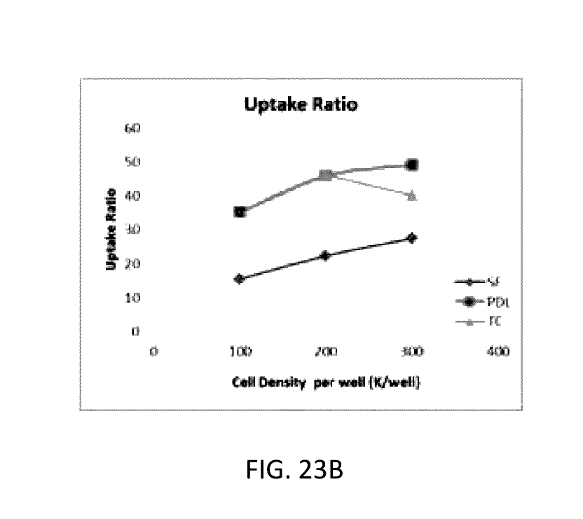

As shown in FIG. 23A, cells cultured in Corning® BioCoat™ PDL 175 cm² Rectangular Straight Neck Cell Culture Flasks with Vented Cap exhibited ˜2-fold higher uptake activity than cells cultured in Erlenmeyer shaker flasks. Additionally, as shown in FIGS. 23A and 23B, the cells cultured in Corning® BioCoat™ PDL 175 cm² Rectangular Straight Neck Cell Culture Flasks with Vented Cap exhibited an overall increase in uptake activity and uptake ratio with increasing cell density (i.e., between 100K cells/well and 300K cells/well). A cell density of 200K cells/well and above formed a suitable sized pellet. Additionally, in all conditions investigated, CV (n=6) between different wells was within 20%.

### Example 5

### Development and Characterization of Assay-Ready Transportocells

Experimental Protocol.

In this experiment, Assay-Ready TransportoCells were developed. Additionally, the effect of culturing conditions on the activity of Organic Anion-Transporting Polypeptide 1B1 in TransportoCells was investigated. With regard to culturing conditions, the effect of culturing media, culturing vessel, culturing time, and the addition of sodium butyrate to a final concentration of 5 mM during culture on the activity of Organic Anion-Transporting Polypeptide 1B1 was investigated.

HEK293 cells (obtained from Life Technologies) were cultured in Corning® erlenmeyer shaker flasks (available from Corning Inc. Life Sciences) using CD293 media (life tech) supplemented with 4 mM L-Glutamine (Gibco) and Penicillin-Streptomycin (10,000 units/ml; available from Gibco Cat. No. 15140-122, Life Technologies Corp On Day 1, about 24 hours before EP, HEK293 cells viability and cell number were determined, as previously described. Then, the cells were centrifuged down. The cell pellet was resuspended in supplemented CD293 media to a final 0.7×10⁶ cells/ml in Corning erlenmeyer shaker flasks. The cells were incubated at 37° C. with 8% CO₂ for 24 hours.

After 24 hours, EP of the cells was executed. HEK293 cells were transiently transfected and recovered using the same EP protocol as described in Example 1. In short, the cells were harvested, cell viability and cell number determined after which cells were pelleted down by spinning at 100 g for 5 min and the media aspirated. Cells were resuspended in EP buffer (obtained from Maxcyte), pelleted down by spinning at 100 g for 5 min, then resuspended in an appropriate amount of EP Buffer (obtained from Maxcyte) to reach a cell density of 100×10⁶ cells/ml (which was used as the cell stock). OATP1B1*1a DNA to be used for EP was prepared in sterile water at a final concentration of 5 mg/ml. For each sample used for OC-400 processing assembly, 0.4 ml of cell stock and OATP1B1*1a DNA was mixed in a sterile 1.5 ml eppendorf tube resulting in a final concentration of 300 μg/ml OATP1B1*1a DNA and cell density of 40×10⁶ cells per sample. For each sample used for CL-2 processing assembly, 10 ml of cell stock and OATP1B1*1a DNA was placed in a 50 ml sterile conical tube resulting in a final concentration of 300 μg/ml OATP1B1*1a DNA.

Samples were transferred into an OC-400 or CL-2 processing assembly (available from MaxCyte, Cat. No. OC-400R and CL2-R, MaxCyte Inc.) which followed the manufacturer's instructions for EP of HEK cells. Following EP, the cells were transferred into Erlenmeyer shaker flasks and incubated for 20 min at 37° C. and 8% CO₂. After 20 min first recovery, supplemented CD293 media was added into the shaker flask to a final 1×10⁶ cells/ml. Cells were further recovered for lhour at 37° C. and 8% CO₂, 100 RPM. After 1 hour recovery, the cells were either cultured in the Erlenmeyer shaker flasks (i.e., cultured in suspension), or were transferred to Corning® BioCoat™ PDL 175 cm² Rectangular Straight Neck Cell Culture Flask with Vented Cap (available from Corning Life Sciences, Cat. No. 354539; hereinafter, “PDL-Treated T-175 Flasks”) or Falcon® 175 cm² Rectangular Straight Neck Cell Culture Flask with Vented Cap (available from Corning life Sciences, Cat. No. 353112; hereinafter, “TC-treated T-175 Flasks”) (i.e., cultured in attached form) for culturing. The culturing conditions employed (i.e., the culturing media, culturing vessel, culturing time, and whether sodium butyrate was added) are detailed in Tables 19-20. The Positive and Negative Controls employed are also detailed in Table 19.

TABLE 19
Culturing Conditions

| | | | Culturing | Sodium |
|---|---|---|---|---|
| | | | Time to | Butyrate (5 mM) |
| Sample | Culturing | Culturing | Harvest | Addition 24 Hours |
| Number | Media | Vessel | (hours) | Prior to Harvest? |
| 1 | CD293 | 40 mL | 24 | Yes |
| 2 | Media | Erlenmeyer | 48 | Yes |
| 3 | | Shaker | | No |
| 4 | | Flasks | 72 | Yes |
| 5 | Plating | PDL- | 24 | Yes |
| 6 | Media | Treated | 48 | No |
| 7 | (containing | T-175 | 48 | Yes |
| 8 | 10% | Flasks | 72 | Yes |
| | FBS) | | | |
| 9 | | TC- | 48 | Yes |
| | | Treated | | |
| | | T-175 | | |
| | | Flasks | | |
| 10 | CD293+ | TC- | 48 | Yes |
| | 10% | Treated | | |
| | FBS | T-175 | | |
| | | Flasks | | |

| | |
|---|---|
| 11 Assay Control: | OATP1B1*1a cells lot 4112001 |
| TransportoCells ™ | (the same batch of OATP1B1*1a, |
| | cryo-freeze immediately post EP |
| 12 Assay Control: | Control Cells lot 4286010 |
| Negative Control | |

TABLE 20
Assay Ready Characterization Conditions

| | Sample Number |
|---|---|
| Conditions | Comparison |
| Transporter Protein Expression Peak Time | 1, 2, 4; 5, 7, 8 |
| (24 hours, 48 hours, 72 hours) | |
| Culture in Suspension versus Culture in Attached Form | 2, 6, 9, 10 |
| and Different Culture Media | |
| (CD293, CD293+10% FBS, Plating Media) | |
| Culture in PDL-Treated T-175 Flask versus TC-treated | 7, 9 |
| T-175 Flask | |
| Sodium Butyrate Effect | 2, 3; 6, 7 |

After the appropriate culturing time, the cells cultured in the Erlenmeyer flasks were harvested via centrifugation at 100 g for 5-10 min, the cells cultured in the PDL-Treated T-175 Flasks were harvested with 0.05% Trypsin (obtained from Sigma), and the cells cultured in the TC-Treated T-175 Flasks were harvested with D-PBS (obtained from Corning). The cells were counted and viability was assessed. The cells were then cryopreserved. For cryopreservation, cells were pelleted down and then resuspended in freshly prepared ice-cold freezing media (9 parts culturing medium and 1 part DMSO which was syringe filtered to sterilize, obtained from Sigma) at a density of 10×10⁶ cells/ml. Cryo vials were filled with 1 ml of this cell suspension, and placed on ice-cold Mr. Frosty freezing container (available from Thermal Scientific) stored in −80° C. freezer overnight after which the vials were stored in liquid nitrogen.

Following cryopreservation, the cells were thawed, counted, and the activity of Organic Anion-Transporting Polypeptide 1B1 was assessed immediately post-thaw following the centrifugation method described in Example 2.

A control was provided via an adherent assay for characterizing the activity of a drug transporter protein in cryopreserved, Corning® TransportoCells™. Corning® TransportoCells™ transiently transfected with the gene OATP1B1*1a were obtained from Corning Life Sciences (Cat. No. 354859). In order to obtain suitable expression of the Organic Anion-Transporting Polypeptide 1B1 encoded by OATP1B1*1a, the Corning® TransportoCells™ were thawed, cultured, and harvested. More specifically, the Corning® TransportoCells™ were thawed in a water bath at 37° C. for about 2 minutes, pelleted down by spinning at 100 g for 5 minutes, and the cell pellet was resuspended in appropriate volume of HBSS buffer (with Ca²⁺ and Mg²⁺, obtained from Corning) at a density of 1×10⁶ cells/ml. The cells were then assayed in the microplate to characterize the activity of Organic Anion-Transporting Polypeptide 1B1 following the centrifugation method described in Example 2.

Results.

As set forth in Table 21 below, cells cultured in Erlenmeyer Shaker Flasks and cells cultured in PDL-Treated T-175 Flasks exhibited a viability of ˜90% and good cell doubling at harvest.

TABLE 21
Characterization of Assay Ready Culturing Conditions*

| | | Culturing | Viability at | Doubling | |
|---|---|---|---|---|---|
| | Culture Media | Vessel | Harvest | at Harvest | Handling** |
| Suspension | CD293 Media | 125 mL | 88.9% | 2.1X | Easy |
| | | Erlenmeyer | | | |
| | | Shaker Flasks | | | |
| Attached | Plating Media | PDL-Treated | 95.40% | 1.5X | Difficult |
| Form | (containing 10% | T-175 Flasks | | | |
| | FBS) | | | | |
| Attached | Plating Media | TC-Treated T- | 60.40% | 0.5X | Medium |
| Form | (containing | 175 Flasks | | | |
| | 10% FBS) | | | | |
| Attached | CD293+10% FBS | TC-Treated T- | 74.60% | 2.1X | Medium |
| Form | Media | 175 Flasks | | | |

*Culturing Time to Harvest - 48 Hours; Sodium Butyrate (5 mM) was added 24 Hours Prior to Harvest; Cells were cryopreserved at a cell density of 10 × 10⁶ cells/ml.
**The handling is rated from easy (#1) to medium (#2) to difficult (#3) for the above culturing conditions respectively.

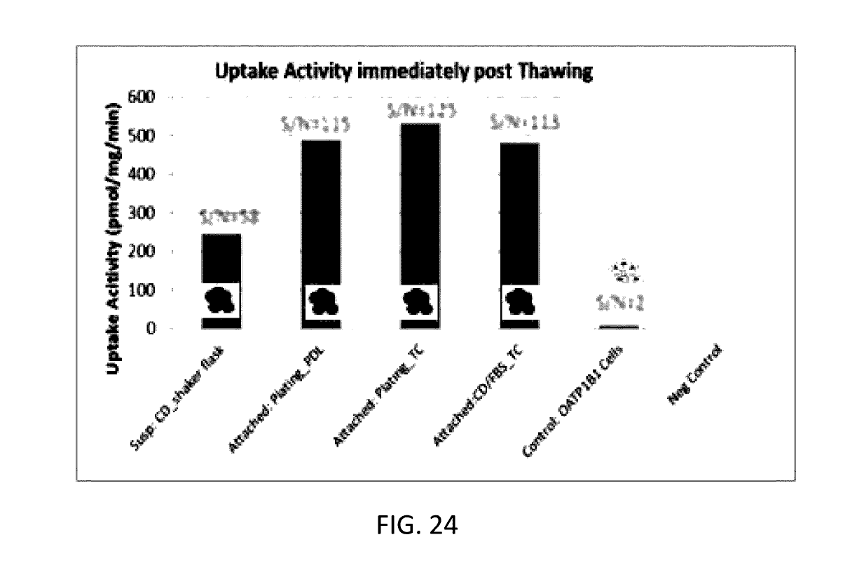

As shown in FIG. 24, the cells cultured in Erlenmeyer Shaker Flasks, PDL-Treated T-175 Flasks, TC-Treated T-175 Flasks with plating media, and TC-Treated T-175 Flasks with CD293 media exhibited an uptake ratio of >50. Additionally, the cells cultured in the attached form (i.e., PDL-Treated T-175 Flasks and TC-Treated T-175 Flasks) exhibited a 2-fold higher uptake activity relative to the cells cultured in suspension (i.e., Erlenmeyer Shaker Flasks). For all conditions, CV was within 15%.

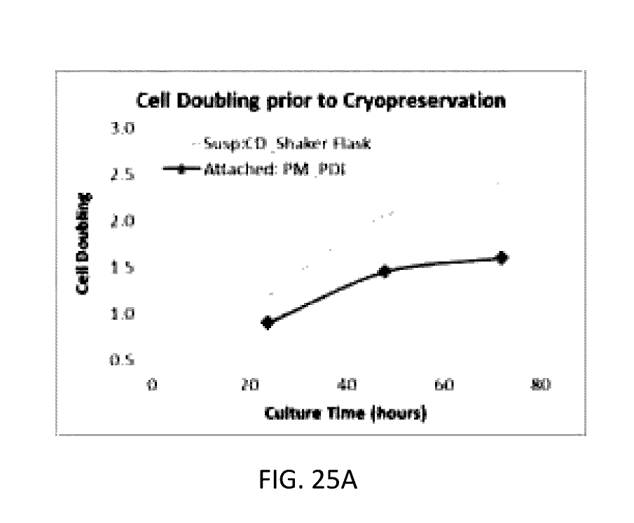

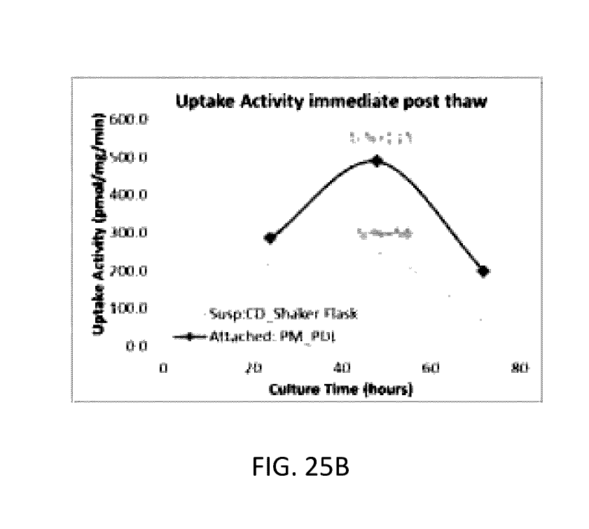

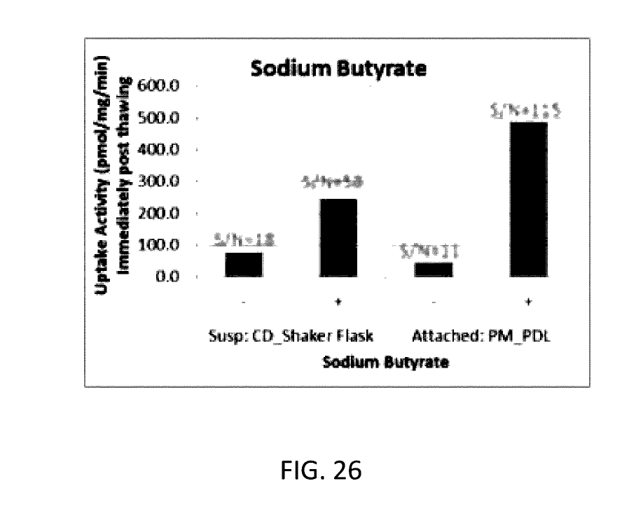

As shown in FIG. 25A, cell doubling increased from 24 hours to 72 hours culturing time for cells cultured in Erlenmeyer Shaker Flasks and for cells cultured in PDL-Treated T-175 Flasks. Additionally, as shown in FIG. 25B, uptake activity immediately post-thaw peaked at 48 hours. Without being bound to the theory, it is believed that culturing time may be adjusted based on the time frame for performing the activity assay. As shown in FIG. 26, uptake activity was boosted by from about 3 fold to 10 fold in cells cultured with 5 mM sodium butyrate as compared to cells cultured without sodium butyrate.

### Example 6

### Further Development of Suspension Assay and Plating Assay for Characterizing Recombinant SLC Transporter Activity in Assay-Ready Transportocells

Experimental Protocol.

In this experiment, assays for characterizing the activity of a drug transporter protein in cryopreserved, Assay-Ready TransportoCells were further developed. More specifically, the timing of performing a suspension assay versus a plating assay in characterizing the activity of Organic Anion-Transporting Polypeptide 1B1 in Assay-Ready TransportoCells was investigated. Cryopreserved, Assay-Ready TransportoCells were manufactured as in Example 4. As in Example 4, the cryopreserved, Assay-Ready TransportoCells were cultured in Erlenmeyer shaker flasks, PDL-Treated T-175 Flasks, or TC-treated T-175 Flasks with plating media or with CD293 media. The assay control and negative control were as described in Example 4.

In a first suspension assay experiment, the cryopreserved, Assay-Ready TransportoCells were thawed in HBSS buffer (obtained from Corning). Then, a suspension assay was conducted to characterize activity of the Organic Anion-Transporting Polypeptide 1B1 encoded by OATP1B1*1a. The suspension assay was conducted using a centrifugation method either immediately following thaw from cryopreservation or 1 hour post-thaw from cryopreservation. Where the suspension assay was conducted 1 hour post-thaw, the cells were incubated at 37° C. in suspension. The suspension assay using the centrifugation method was as described in Example 2.

In a second plating assay experiment, the cryopreserved, Assay-Ready TransportoCells were thawed in plating media (obtained from Table 16). The cells were plated on PDL treated 24 well plate (Corning) and incubated for 4 hours, allowing the cells to attach to the plate. Then, a plate assay was conducted to characterize activity of the Organic Anion-Transporting Polypeptide 1B1 encoded by OATP1B1*1a. The plate assay was conducted 4 hours post-thaw from cryopreservation.

Results.

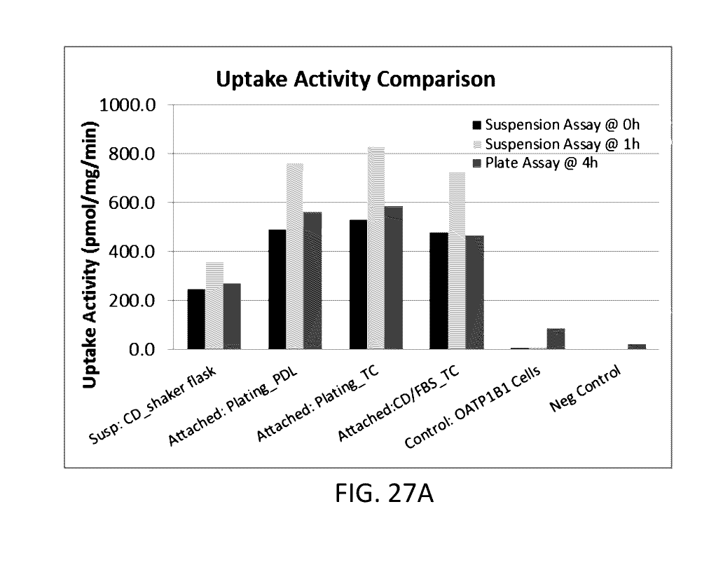

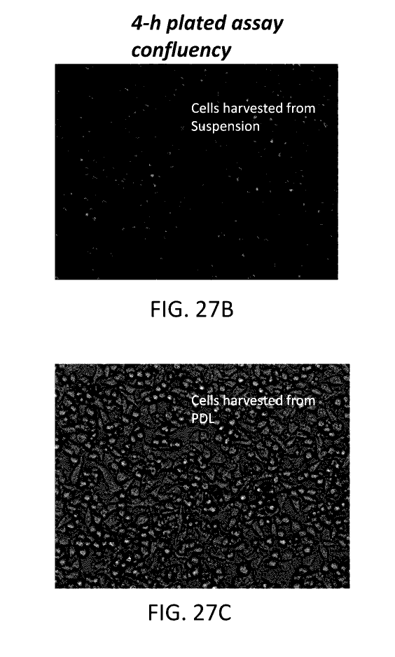

As shown in FIG. 27, cells assayed for activity at 1 hour post-thaw via suspension assay exhibited increased uptake activity relative to suspension assays conducted for activity at 0 hours post-thaw. Without being bound by the theory, it is believed that performance of the suspension assay at 1 hour post-thaw allowed the cells to recover from cryopreservation. Further, cells assayed for activity at 4 hours post-thaw via plate assay exhibited comparable (slightly higher) uptake activity relative to cells assayed for activity via suspension assay at 0 hours post-thaw.

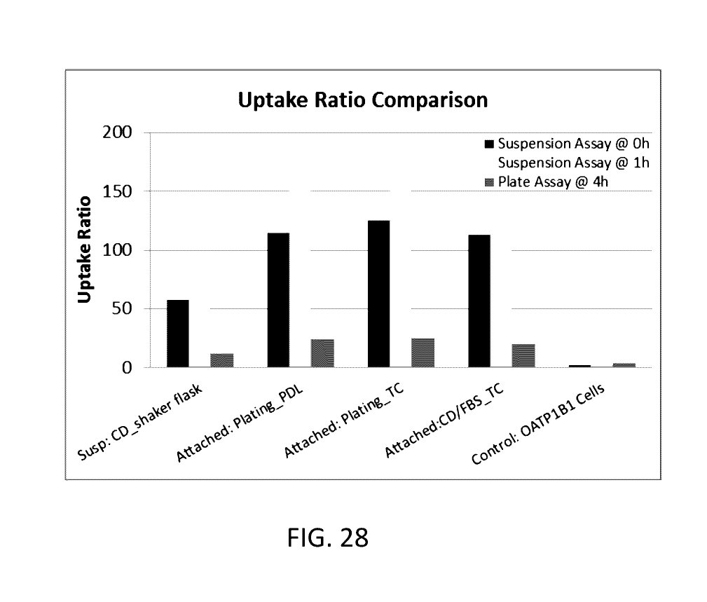

Of note, as shown in FIG. 28, cells assayed for activity via suspension assays exhibited higher uptake ratio relative to cells assayed for activity at 4 hours post-thaw via plate assay. Specifically, the uptake ratio of cells assayed for activity via suspension assays was from about 50 to 150. In contrast, the uptake ratio of cells assayed for activity at 4 hours post-thaw via plate assay was from about 10 to about 30.

### Example 7

### Characterization of Effect of Thawing Media on SLC Transporter Activity in Assay-Ready Transportocells

Experimental Protocol.

In this experiment, the effect of thawing media on the activity of Organic Anion-Transporting Polypeptide 1B1 in Assay-Ready TransportoCells was investigated. Cryopreserved, Assay-Ready TransportoCells were made as in Example 4. As in Example 4, the cryopreserved, Assay-Ready TransportoCells were cultured in Erlenmeyer shaker flasks, PDL-Treated T-175 Flasks, or TC-treated T-175 Flasks with plating media or with CD293 media. The assay control and negative control were as described in Example 4.

In a first thaw media experiment, the cryopreserved, Assay-Ready TransportoCells were thawed in HBSS buffer (obtained from Corning), pelleted down, and resuspended in HBSS. Viability was assessed and cells were counted. In a second thaw media experiment, the cryopreserved, Assay-Ready TransportoCells were thawed in plating media (obtained from Table 16), pelleted down, and resuspended in plating media. Cell viability and cell number were determined, as previously described.

Results.

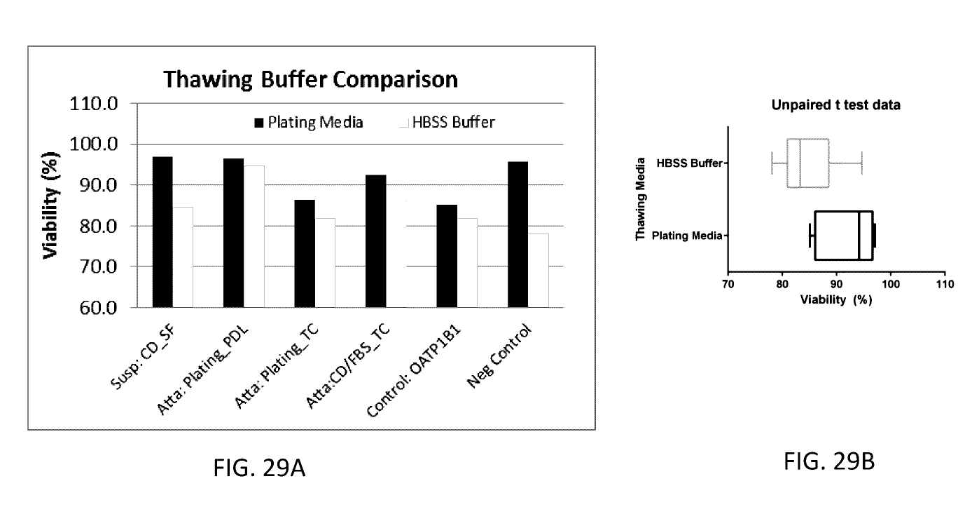

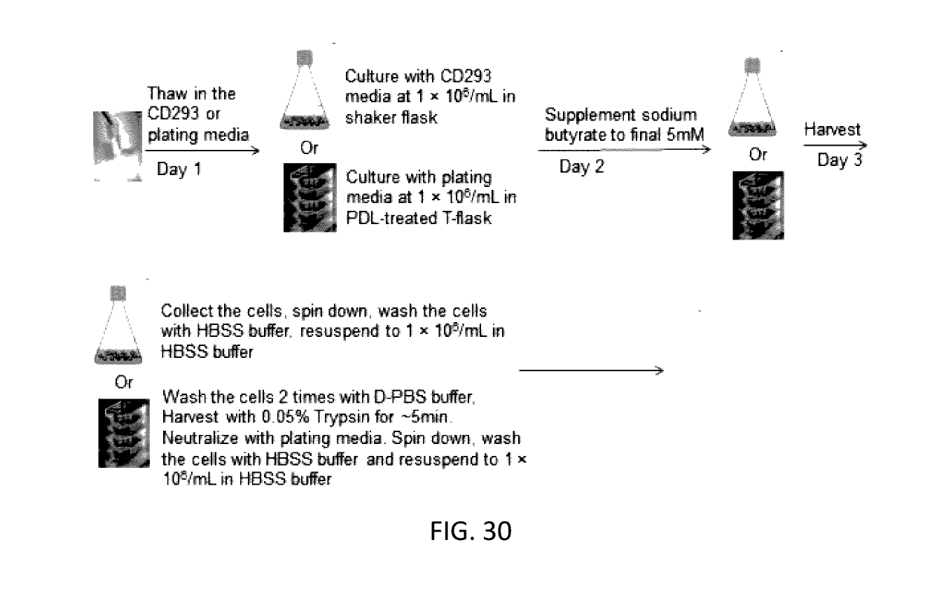

As shown in FIG. 29, the cells thawed in Plating Media exhibited significantly higher viability as compared to cells thawed in HBSS Buffer (n=6, p=0.0245<0.05). Appropriate culturing conditions are depicted in FIG. 30.

It will be appreciated by those skilled in the art that changes could be made to the embodiments described above without departing from the broad inventive concept thereof. It is understood, therefore, that this disclosure is not limited to the particular embodiments disclosed, but is intended to cover modifications that are within the spirit and scope of the disclosure, as defined by the appended claims.

All documents cited are incorporated herein by reference; the citation of any document is not to be construed as an admission that it is prior art with respect to the present disclosure.

It is to be further understood that where descriptions of various embodiments use the term “comprising,” and/or “including” those skilled in the art would understand that in some specific instances, an embodiment can be alternatively described using language “consisting essentially of” or “consisting of.”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all technical and scientific terms used herein have the same meaning as commonly understood by one of ordinary skill in the art to which the claimed subject matter belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

What is claimed is:

1. A recombinant cell comprising one or more transiently overexpressed genes encoding a drug transporter protein, wherein:
the recombinant cell is cryopreserved, and
activity of the drug transporter protein is detectable in a population of the recombinant cells prior to cyropreservation at an uptake ratio of at least 5.

2. The recombinant cell of claim 1, wherein the activity of the drug transporter protein would be detectable in a population of the recombinant cells following thaw from cryopreservation at an uptake ratio of at least 5.

3. The recombinant cell of claim 2, wherein the activity of the drug transporter protein would be detectable in the population of the recombinant cells following thaw from cryopreservation at an uptake ratio of from about 5 to about 150.

4. The recombinant cell of claim 1, wherein:
the activity of the drug transporter protein would be detectable in a plated population of the recombinant cells following thaw from cryopreservation at an uptake ratio of from about 5 to about 30 within 4 hours of thawing.

5. The recombinant cell of claim 1, wherein:
the activity of the drug transporter protein would be detectable in a suspended population of the recombinant cells following thaw from cryopreservation at an uptake ratio of from about 5 to about 150 within 1 hour of thawing.

6. The recombinant cell of claim 1, wherein the drug transporter protein is selected from the group consisting of an ATP binding cassette transporter and a solute carrier transporter protein.

7. The recombinant cell of claim 6, wherein the one or more transiently overexpressed genes is selected from the group consisting of MDR1/Mdr1a/Mdr1b, MRP1/Mrp1, MRP2/Mrp2, MRP3/Mrp3, MRP4/Mrp4, MRP5/Mrp5, MRP6/Mrp6, MRP7/Mrp7, MRP 8/Mrp8, BCRP/Bcrp, BSEP/Bsep, OATP2/Oatp2, OATP1B3/Oatp1b3, OAT1/Oat1, OAT2/Oat2, OAT3/Oat3, OAT4/Oat4, OCT1/Oct1, OCT2/Oct2, OATP1/Oatp1, PEPT1/Pept1, PEPT2/Pept2, OCTN1/Octn1, OCTN2/Octn2, MATE1/Mate1, MATE2K/Mate2, URAT1/Urat1, ASBT/Asbt, NTCP/Ntcp, and a combination thereof.

8. The recombinant cell of claim 6, wherein the one or more transiently overexpressed genes is selected from the group consisting of OATP2/Oatp2, OATP1B3/Oatp1b3, OAT1/Oat1, OAT2/Oat2, OAT3/Oat3, OAT4/Oat4, OCT1/Oct1, OCT2/Oct2, OATP1/Oatp1, PEPT1/Pept1, PEPT2/Pept2, OCTN1/Octn1, OCTN2/Octn2, MATE1/Mate1, MATE2K/Mate2, URAT1/Urat1, ASBT/Asbt, NTCP/Ntcp, and a combination thereof.

9. The recombinant cell of claim 8, wherein the OATP2/Oatp2 is selected from the group consisting of OATP1B1*1a, OATP1B1*1b, OATP1B1*5, OATP1B1*15, and combinations thereof.

10. The recombinant cell of claim 9, wherein said OATP2/Oatp2 is OATP1B1*1a.

11. The recombinant cell of claim 9, wherein said OATP2/Oatp2 is OATP1B1*1b.

12. The recombinant cell of claim 9, wherein said OATP2/Oatp2 is OATP1B1*5.

13. The recombinant cell of claim 9, wherein said OATP2/Oatp2 is OATP1B1*15.

14. The recombinant cell of claim 1, wherein the one or more transiently overexpressed genes is derived individually from a human or an animal species selected from the group consisting of a mouse, a rat, a guinea pig, a dog, and a monkey.

15. The recombinant cell of claim 14, wherein the one or more transiently overexpressed genes encodes a solute carrier transporter protein selected from the group consisting of monkety Oatp1b1, monkey Oatp1b3, dog Oatp1b4, rat Oatp1b2, rat Oatp1a1, rat Oatp1a4, and combinations thereof.

16. The recombinant cell of claim 1, wherein the cell comprises a hepatocyte.

17. The recombinant cell of claim 1, wherein the cell comprises an endothelial cell.

18. A process of preparing cryopreserved transiently transfected recombinant cells, the process comprising:
transiently transfecting cells with one or more genes encoding a drug transporter protein to provide the transiently transfected recombinant cells, and
cryopreserving the transiently transfected recombinant cells within 48 hours of transfection, wherein a population of the transiently transfected recombinant cells transiently overexpress the one or more genes encoding the drug transporter protein at a detectable level prior to cryopreserving the transiently transfected recombinant cells, and wherein the detectable level prior to cryopreserving is an uptake ratio of at least 5.

19. The process of claim 18, wherein transient transfection of the cells comprises electroporation.

20. The process of claim 18, wherein the transiently transfected recombinant cells are cryopreserved at about 24 hours to about 48 hours post transfection.

21. The process of claim 18, wherein:
a population of the transiently transfected recombinant cells would overexpress the one or more genes encoding the drug transporter protein at the detectable level following thaw from cryopreservation, and
the detectable level following thaw from cryopreservation is an uptake ratio of at least 5.

22. The process of claim 18, wherein:
a suspended population of the transiently transfected recombinant cells would overexpress the one or more genes encoding the drug transporter protein at the detectable level following thaw from cryopreservation within 1 hour post thaw.

23. The process of claim 22, wherein the detectable level following thaw from cryopreservation is an uptake ratio of from about 5 to about 150.

24. The process of claim 18, wherein:
a plated population of the transiently transfected recombinant cells would overexpress the one or more genes encoding the drug transporter protein at the detectable level following thaw from cryopreservation within 4 hours post thaw.

25. The process of claim 24, wherein the detectable level following thaw from cryopreservation is an uptake ratio of from about 5 to about 30.
